(19) United States
(12) Patent Application Publication
(10) Pub. No.: US 2024/0293582 A1
(43) Pub. Date: Sep. 5, 2024

(54) DNA COMPOSITIONS COMPRISING MODIFIED CYTOSINE

(71) Applicant: FLAGSHIP PIONEERING INNOVATIONS VII, LLC, Cambridge, MA (US)

(72) Inventors: Jacob Rosenblum Rubens, Cambridge, MA (US); Eric Christopher Keen, Cambridge, MA (US); Alexandra Rachael Sneider, Cambridge, MA (US); Jeffrey Tsao, Charlestown, MA (US); Camilo Ayala Breton, Andover, MA (US); Carl Wayne Brown, III, Westborough, MA (US); Samuel Alves-Czachor, Somerville, MA (US); Edward Matthew Kennedy, Bedford, MA (US)

(21) Appl. No.: 18/444,222

(22) Filed: Feb. 16, 2024

Related U.S. Application Data
(60) Provisional application No. 63/594,806, filed on Oct. 31, 2023.
(60) Provisional application No. 63/485,787, filed on Feb. 17, 2023.

(51) Int. Cl. A61K 48/00; C12N 15/11

(52) U.S. Cl. CPC A61K 48/0066; C12N 15/11; C12N 2310/334

(57) ABSTRACT

This disclosure provides, for example, double stranded DNA (dsDNA) molecules comprising a chemically modified cytosine nucleotide. In some embodiments, the dsDNA molecules comprise a therapeutic payload sequence. In some embodiments, the dsDNA molecules are resistant to endonuclease digestion and/or resistant to immune sensor recognition, and supports expression of a therapeutic payload encoded in the dsDNA molecules. The disclosure also provides, for example, pharmaceutical compositions comprising dsDNA molecules comprising a chemically modified cytosine nucleotide.

### RELATED APPLICATIONS

This application claims priority to U.S. Ser. No. 63/485,787, filed Feb. 17, 2023, and U.S. Ser. No. 63/594,806, filed Oct. 31, 2023, the entire contents of each of which are incorporated herein by reference.

### SEQUENCE LISTING

The instant application contains a Sequence Listing which has been submitted electronically in XML format and is hereby incorporated by reference in its entirety. Said XML copy, created on Feb. 6, 2024, is named F2128-7007WO_SL.xml and is 73,983 bytes in size.

### BACKGROUND

There is a need for novel therapeutic modalities to address unmet medical need.

### SUMMARY OF THE INVENTION

Described herein are pharmaceutical DNA compositions, constructs, preparations, methods of using such compositions, constructs and preparations, and methods of making the same.

### Enumerated Embodiments

1. A double stranded DNA (dsDNA) molecule comprising a chemically modified cytosine nucleotide having a substitution other than hydrogen at carbon 5 of the cytosine.

2. The dsDNA molecule of embodiment 1, wherein the chemically modified cytosine nucleotide comprises the structure of Formula I:

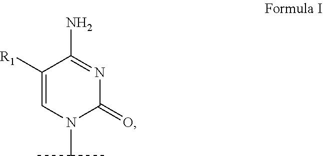

wherein R₁ is selected from the group consisting of —OH; -aldehyde; -carboxylic acid; -alkyl; —(CH₂)ₘOR₂, m=1-3 and R₂=H or a sugar molecule; and -propargylamino.

3. A dsDNA molecule comprising:
a promoter sequence and a therapeutic payload sequence operably linked to the promoter sequence, and
a chemically modified cytosine nucleotide situated in the therapeutic payload sequence comprising the structure of Formula I:

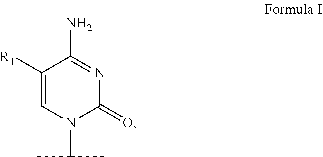

wherein R₁ is selected from the group consisting of —OH; -aldehyde; -carboxylic acid; -alkyl; —(CH₂)ₘOR₂, m=1-3 and R₂=H or a sugar molecule; and -propargylamino.

4. The dsDNA molecule of embodiment 2 or 3, wherein R₁ is selected from the group consisting of —OH; —CHO; —COOH; -alkyl; —(CH₂)ₘOR₂, m=1-3 and R₂=H or a sugar molecule; and -propargylamino, wherein the alkyl group includes one to six carbons.

5. The dsDNA molecule of any of embodiments 2-4, wherein R₁ is selected from the group consisting of —OH; —CHO; —COOH; —CH₂OR₃, R₃=H or glucose; -methyl; and -propargylamino.

6. The dsDNA molecule of any of the preceding embodiments, wherein the chemically modified cytosine nucleotide comprises 5-formylcytosine, 5-hydroxycytosine, 5-carboxycytosine, 5-propargylaminocytosine, 5-methylcytosine, 5-hydroxymethylcytosine, or glucosyl-5-hydroxymethylcytosine.

7. The dsDNA molecule of any of embodiments 1-6, wherein the chemically modified cytosine nucleotide comprises 5-formylcytosine.

8. The dsDNA molecule of any of embodiment 1-6, wherein the chemically modified cytosine nucleotide comprises 5-hydroxycytosine.

9. A double stranded DNA (dsDNA) molecule comprising a chemically modified cytosine nucleotide, wherein the chemically modified cytosine nucleotide comprises 5-hydroxycytosine.

10. The dsDNA molecule of any of embodiments 1-6, wherein the chemically modified cytosine nucleotide comprises 5-carboxycytosine.

11. The dsDNA molecule of any of embodiments 1-6, wherein the chemically modified cytosine nucleotide comprises 5-propargylaminocytosine.

12. A double stranded DNA (dsDNA) molecule comprising a chemically modified cytosine nucleotide, wherein the chemically modified cytosine nucleotide comprises 5-propargylaminocytosine.

13. The dsDNA molecule of any of embodiments 1-6, wherein the chemically modified cytosine nucleotide comprises 5-methylcytosine.

14. The dsDNA molecule of any of embodiments 1-6, wherein the chemically modified cytosine nucleotide comprises 5-hydroxymethylcytosine.

15. The dsDNA molecule of any of embodiments 1-6, wherein the chemically modified cytosine nucleotide comprises glucosyl-5-hydroxymethylcytosine.

16. A double stranded DNA (dsDNA) molecule comprising a chemically modified cytosine nucleotide, wherein the chemically modified cytosine nucleotide comprises glucosyl-5-hydroxymethylcytosine.

17. A double stranded DNA (dsDNA) molecule comprising:
a chemically modified cytosine nucleotide chosen from 5-hydroxycytosine or glucosyl-5-hydroxymethylcytosine,
wherein the dsDNA molecule is closed-ended linear DNA.

18. A double stranded DNA (dsDNA) molecule comprising:
a promoter sequence and a therapeutic payload sequence operably linked to the promoter sequence, and
a chemically modified cytosine nucleotide which is 5-hydroxycytosine, situated in the therapeutic payload sequence,
wherein the dsDNA molecule is closed-ended linear DNA.

19. The dsDNA molecule of any of embodiments 1-18, wherein the dsDNA molecule is circular or linear.

20. The dsDNA molecule of any of the preceding embodiments, wherein the dsDNA molecule is circular.

21. The dsDNA molecule of any of embodiments 1-19, wherein the dsDNA molecule is linear.

22. The dsDNA molecule of any of embodiments 1-19 or 21, wherein the dsDNA molecule is closed-ended linear.

23. The dsDNA molecule of any of the preceding embodiments, wherein at least 1%, at least 5%, at least 10%, at least 15%, at least 20%, at least 25%, at least 50%, or at least 75% of cytosine positions in the dsDNA molecule comprise the chemically modified cytosine nucleotide.

24. A double stranded DNA (dsDNA) molecule comprising a chemically modified cytosine nucleotide, wherein at least 1%, at least 5%, at least 10%, at least 15%, at least 20%, at least 25%, at least 50%, or at least 75% of cytosine positions in the dsDNA molecule comprise the chemically modified cytosine nucleotide.

25. The dsDNA molecule of any of the preceding embodiments, wherein 1%-75% (e.g., 1-5%, 5-10%, 10-15%, 15-20%, 20-25%, 25-30%, 30-35%, 35-40%, 40-45%, 45-50%, 50-55%, 55-60%, 60-65%, 65-70%, or 70-75%) of cytosine positions in the dsDNA molecule comprise the chemically modified cytosine nucleotide.

26. A double stranded DNA (dsDNA) molecule comprising a chemically modified cytosine nucleotide, wherein 1%-75% (e.g., 1-5%, 5-10%, 10-15%, 15-20%, 20-25%, 25-30%, 30-35%, 35-40%, 40-45%, 45-50%, 50-55%, 55-60%, 60-65%, 65-70%, or 70-75%) of cytosine positions in the dsDNA molecule comprise the chemically modified cytosine nucleotide.

27. The dsDNA molecule of any of embodiments 1-26, wherein 1%-75% (e.g., 1-5%, 5-10%, 10-15%, 15-20%, 20-25%, 25-30%, 30-35%, 35-40%, 40-45%, 45-50%, 50-55%, 55-60%, 60-65%, 65-70%, or 70-75%) of cytosine positions in the dsDNA molecule comprise the chemically modified cytosine nucleotide, wherein the chemically modified cytosine nucleotide comprises 5-formylcytosine.

28. The dsDNA molecule of any of embodiments 1-27, wherein at least 1%, at least 5%, at least 10%, at least 15%, at least 20%, at least 25%, at least 50%, or at least 75% of cytosine positions in the dsDNA molecule comprise the chemically modified cytosine nucleotide, wherein the chemically modified cytosine nucleotide comprises 5-formylcytosine.

29. The dsDNA molecule of any of embodiments 1-26, wherein 1%-75% (e.g., 1-5%, 5-10%, 10-15%, 15-20%, 20-25%, 25-30%, 30-35%, 35-40%, 40-45%, 45-50%, 50-55%, 55-60%, 60-65%, 65-70%, or 70-75%) of cytosine positions in the dsDNA molecule comprise the chemically modified cytosine nucleotide, wherein the chemically modified cytosine nucleotide comprises 5-hydroxycytosine.

30. The dsDNA molecule of any of embodiments 1-26 or 28, wherein at least 1%, at least 5%, at least 10%, at least 15%, at least 20%, at least 25%, at least 50%, or at least 75% of cytosine positions in the dsDNA molecule comprise the chemically modified cytosine nucleotide, wherein the chemically modified cytosine nucleotide comprises 5-hydroxycytosine.

31. The dsDNA molecule of any of embodiments 1-26, wherein 1%-75% (e.g., 1-5%, 5-10%, 10-15%, 15-20%, 20-25%, 25-30%, 30-35%, 35-40%, 40-45%, 45-50%, 50-55%, 55-60%, 60-65%, 65-70%, or 70-75%) of cytosine positions in the dsDNA molecule comprise the chemically modified cytosine nucleotide, wherein the chemically modified cytosine nucleotides comprise 5-carboxycytosine.

32. The dsDNA molecule of any of embodiments 1-26 or 31, wherein at least 1%, at least 5%, at least 10%, at least 15%, at least 20%, at least 25%, at least 50%, or at least 75% of cytosine positions in the dsDNA molecule comprise the chemically modified cytosine nucleotide, wherein the chemically modified cytosine nucleotide comprises 5-carboxycytosine.

33. The dsDNA molecule of any of embodiments 1-26, wherein 1%-75% (e.g., 1-5%, 5-10%, 10-15%, 15-20%, 20-25%, 25-30%, 30-35%, 35-40%, 40-45%, 45-50%, 50-55%, 55-60%, 60-65%, 65-70%, or 70-75%) of cytosine positions in the dsDNA molecule comprise the chemically modified cytosine nucleotide, wherein the chemically modified cytosine nucleotide comprises 5-propargylaminocytosine.

34. The dsDNA molecule of any of embodiments 1-26 or 33, wherein at least 1%, at least 5%, at least 10%, at least 15%, at least 20%, at least 25%, at least 50%, or at least 75% of cytosine positions in the dsDNA molecule comprise the chemically modified cytosine nucleotide, wherein the chemically modified cytosine nucleotide comprises 5-propargylaminocytosine.

35. The dsDNA molecule of any of embodiments 1-26, wherein 1%-75% (e.g., 1-5%, 5-10%, 10-15%, 15-20%, 20-25%, 25-30%, 30-35%, 35-40%, 40-45%, 45-50%, 50-55%, 55-60%, 60-65%, 65-70%, or 70-75%) of cytosine positions in the dsDNA molecule comprise the chemically modified cytosine nucleotide, wherein the chemically modified cytosine nucleotide comprises 5-methylcytosine.

36. The dsDNA molecule of any of embodiments 1-26 or 35, wherein at least 1%, at least 5%, at least 10%, at least 15%, at least 20%, at least 25%, at least 50%, or at least 75% of cytosine positions in the dsDNA molecule comprise the chemically modified cytosine nucleotide, wherein the chemically modified cytosine nucleotide comprises 5-methylcytosine.

37. The dsDNA molecule of any of embodiments 1-26, wherein 1%-75% (e.g., 1-5%, 5-10%, 10-15%, 15-20%, 20-25%, 25-30%, 30-35%, 35-40%, 40-45%, 45-50%, 50-55%, 55-60%, 60-65%, 65-70%, or 70-75%) of cytosine positions in the dsDNA molecule comprise the chemically modified cytosine nucleotide, wherein the chemically modified cytosine nucleotide comprises 5-hydroxymethylcytosine.

38. The dsDNA molecule of any of embodiments 1-26 or 37, wherein at least 1%, at least 5%, at least 10%, at least 15%, at least 20%, at least 25%, at least 50%, or at least 75% of cytosine positions in the dsDNA molecule comprise the chemically modified cytosine nucleotide, wherein the chemically modified cytosine nucleotide comprises 5-hydroxymethylcytosine.

39. The dsDNA molecule of any of embodiments 1-26, wherein 1%-75% (e.g., 1-5%, 5-10%, 10-15%, 15-20%, 20-25%, 25-30%, 30-35%, 35-40%, 40-45%, 45-50%, 50-55%, 55-60%, 60-65%, 65-70%, or 70-75%) of cytosine positions in the dsDNA molecule comprise the chemically modified cytosine nucleotide, wherein the chemically modified cytosine nucleotides comprise glucosyl-5-hydroxymethylcytosine.

40. The dsDNA molecule of any of embodiments 1-26 or 39, wherein at least 1%, at least 5%, at least 10%, at least 15%, at least 20%, at least 25%, at least 50%, or at least 75% of cytosine positions in the dsDNA molecule comprise the chemically modified cytosine nucleotide, wherein the chemically modified cytosine nucleotide comprises glucosyl-5-hydroxymethylcytosine.

41. The dsDNA molecule of any of embodiments 1-40, which comprises a contiguous region of 200 nucleotides in which at least 1%, at least 5%, at least 10%, at least 15%, at least 20%, at least 25%, at least 50%, or at least 75% of cytosine positions in the contiguous region comprise the chemically modified cytosine nucleotide.

42. The dsDNA molecule of any of embodiments 1-41, which comprises a contiguous region of 500 nucleotides in which at least 1%, at least 5%, at least 10%, at least 15%, at least 20%, at least 25%, at least 50%, or at least 75% of cytosine positions in the contiguous region comprise the chemically modified cytosine nucleotide.

43. The dsDNA molecule of any of embodiments 1-42, which comprises a contiguous region of 1000 nucleotides in which at least 1%, at least 5%, at least 10%, at least 15%, at least 20%, at least 25%, at least 50%, or at least 75% of cytosine positions in the contiguous region comprise the chemically modified cytosine nucleotide.

44. The dsDNA molecule of any of embodiments 1-43, which comprises a contiguous region of 200 nucleotides in which 1%-75% (e.g., 1-5%, 5-10%, 10-15%, 15-20%, 20-25%, 25-30%, 30-35%, 35-40%, 40-45%, 45-50%, 50-55%, 55-60%, 60-65%, 65-70%, or 70-75%) of cytosine positions in the contiguous region comprise the chemically modified cytosine nucleotide.

45. The dsDNA molecule of any of embodiments 1-44, which comprises a contiguous region of 500 nucleotides in which 1%-75% (e.g., 1-5%, 5-10%, 10-15%, 15-20%, 20-25%, 25-30%, 30-35%, 35-40%, 40-45%, 45-50%, 50-55%, 55-60%, 60-65%, 65-70%, or 70-75%) of cytosine positions in the contiguous region comprise the chemically modified cytosine nucleotide.

46. The dsDNA molecule of any of embodiments 1-45, which comprises a contiguous region of 1000 nucleotides in which 1%-75% (e.g., 1-5%, 5-10%, 10-15%, 15-20%, 20-25%, 25-30%, 30-35%, 35-40%, 40-45%, 45-50%, 50-55%, 55-60%, 60-65%, 65-70%, or 70-75%) of cytosine positions in the contiguous region comprise the chemically modified cytosine nucleotide.

47. The dsDNA molecule of any of embodiments 1-46, wherein the dsDNA molecule comprises a sense strand and an antisense strand, and wherein the antisense strand comprises fewer chemically modified cytosine nucleotides than the sense strand contains.

48. The dsDNA molecule of any of embodiments 1-47, wherein the dsDNA molecule comprises a sense strand and an antisense strand, and wherein the antisense strand is substantially free of chemically modified cytosine nucleotides.

49. The dsDNA molecule of any of the preceding embodiments, wherein the dsDNA molecule further comprises a second chemically modified cytosine nucleotide.

50. The dsDNA molecule of embodiment 49, wherein:
i) the chemically modified cytosine nucleotide comprises 5-formylcytosine and the second chemically modified cytosine nucleotide comprises 5-hydroxycytosine;
ii) the chemically modified cytosine nucleotide comprises 5-formylcytosine and the second chemically modified cytosine nucleotide comprises 5-carboxycytosine;
iii) the chemically modified cytosine nucleotide comprises 5-formylcytosine and the second chemically modified cytosine nucleotide comprises 5-propargylaminocytosine;
iv) the chemically modified cytosine nucleotide comprises 5-formylcytosine and the second chemically modified cytosine nucleotide comprises 5-methylcytosine;
v) the chemically modified cytosine nucleotide comprises 5-formylcytosine and the second chemically modified cytosine nucleotide comprises 5-hydroxymethylcytosine;
vi) the chemically modified cytosine nucleotide comprises 5-formylcytosine and the second chemically modified cytosine nucleotide comprises glucosyl-5-hydroxymethylcytosine; vii) the chemically modified cytosine nucleotide comprises 5-hydroxycytosine and the second chemically modified cytosine nucleotide comprises 5-carboxycytosine;
viii) the chemically modified cytosine nucleotide comprises 5-hydroxycytosine and the second chemically modified cytosine nucleotide comprises 5-propargylaminocytosine;
ix) the chemically modified cytosine nucleotide comprises 5-hydroxycytosine and the second chemically modified cytosine nucleotide comprises 5-methylcytosine;
x) the chemically modified cytosine nucleotide comprises 5-hydroxycytosine and the second chemically modified cytosine nucleotide comprises 5-hydroxymethylcytosine;
xi) the chemically modified cytosine nucleotide comprises 5-hydroxycytosine and the second chemically modified cytosine nucleotide comprises glucosyl-5-hydroxymethylcytosine;
xii) the chemically modified cytosine nucleotide comprises 5-carboxycytosine and the second chemically modified cytosine nucleotide comprises 5-propargylaminocytosine;
xiii) the chemically modified cytosine nucleotide comprises 5-carboxycytosine and the second chemically modified cytosine nucleotide comprises 5-methylcytosine;
xiv) the chemically modified cytosine nucleotide comprises 5-carboxycytosine and the second chemically modified cytosine nucleotide comprises 5-hydroxymethylcytosine;
xv) the chemically modified cytosine nucleotide comprises 5-carboxycytosine and the second chemically modified cytosine nucleotide comprises glucosyl-5-hydroxymethylcytosine;
xvi) the chemically modified cytosine nucleotide comprises 5-propargylaminocytosine and the second chemically modified cytosine nucleotide comprises 5-methylcytosine;
xvii) the chemically modified cytosine nucleotide comprises 5-propargylaminocytosine and the second chemically modified cytosine nucleotide comprises 5-hydroxymethylcytosine;
xviii) the chemically modified cytosine nucleotide comprises 5-propargylaminocytosine and the second chemically modified cytosine nucleotide comprises glucosyl-5-hydroxymethylcytosine;
xix) the chemically modified cytosine nucleotide comprises 5-methylcytosine and the second chemically modified cytosine nucleotide comprises 5-hydroxymethylcytosine;
xx) the chemically modified cytosine nucleotide comprises 5-methylcytosine and the second chemically modified cytosine nucleotide comprises glucosyl-5-hydroxymethylcytosine; or
xxi) the chemically modified cytosine nucleotide comprises 5-hydroxymethylcytosine and the second chemically modified cytosine nucleotide comprises glucosyl-5-hydroxymethylcytosine.

51. The dsDNA molecule of any of the preceding embodiments, wherein at least 80%, at least 85%, at least 90%, at least 95%, at least 98%, at least 99%, or 100% of the sugars of the dsDNA molecule are deoxyribose sugars.

52. The dsDNA molecule of any of the preceding embodiments, wherein at least 80%, at least 85%, at least 90%, at least 95%, at least 98%, at least 99%, or 100% of the sugars of the chemically modified cytosine nucleotides of the dsDNA molecule are deoxyribose sugars.

53. The dsDNA molecule of any of the preceding embodiments, wherein all positions in the dsDNA molecule comprise a deoxyribose sugar.

54. The dsDNA molecule of any of the preceding embodiments, which comprises a chemical modification of a phosphate group.

55. The dsDNA molecule of any of the preceding embodiments, which comprises a chemical modification of a sugar, e.g., a 2′-deoxy-2′-fluoro (2′-F) nucleotide or a 2′-O-methyl (2′-O-Me) nucleotide.

56. The dsDNA molecule of any of the preceding embodiments, which comprises one or more of:
i) a promoter sequence (wherein optionally the promoter sequence is in the double stranded region);
ii) a payload sequence (e.g., a therapeutic payload sequence) operably linked to the promoter sequence;
iii) a heterologous functional sequence, e.g., a nuclear targeting sequence or a regulatory sequence;
iv) a maintenance sequence; and/or
v) an origin of replication.

57. The dsDNA molecule of any of the preceding embodiments, which comprises one, two, or all of:
i) a heterologous functional sequence, e.g., a nuclear targeting sequence or a regulatory sequence;
ii) a maintenance sequence; or
iii) an origin of replication.

58. The dsDNA molecule of any of the preceding embodiments, which comprises a therapeutic payload sequence.

59. The dsDNA molecule of embodiment 58, wherein the chemically modified cytosine nucleotide is situated in the sense strand of the therapeutic payload sequence.

60. The dsDNA molecule of any of embodiments 1-20 or 21-59, wherein the dsDNA molecule is linear and comprises:
a) an upstream exonuclease-resistant DNA end form;
b) a double stranded region; and
c) a downstream exonuclease-resistant DNA end form.

61. The dsDNA molecule of any of the preceding embodiments, which, when contacted to HEKa cells, e.g., in an assay as described herein, results in one or more of:
a lower level of IFNβ mRNA compared to a control DNA molecule (e.g., at least 10%, at least 20%, at least 30%, at least 40%, at least 50%, at least 60%, at least 70%, at least 80%, or at least 90% lower),
a lower level of CXCL10 mRNA compared to a control DNA molecule (e.g., at least 10%, at least 20%, at least 30%, at least 40%, at least 50%, at least 60%, at least 70%, at least 80%, at least 90%, or at least 95% lower), or
a lower level of IL6 mRNA compared to a control DNA molecule (e.g., at least 10%, at least 20%, or at least 30% lower),
wherein the control DNA molecule comprises the same sequence, same strandedness, and same circular or linear character as the dsDNA molecule, but comprises unmodified cytosine nucleotides in place of the chemically modified cytosine nucleotides.

62. The dsDNA molecule of any of the preceding embodiments, which, when contacted to HEKa cells, e.g., in an assay as described herein, results in one or both of:
(i) a reduction of a measure of interferon signaling relative to a control DNA molecule, e.g., at least a 2-, at least a 4-, at least a 5- or at least a 6-fold reduction, wherein the measure of interferon signaling is an average fold-change of IFNβ mRNA and CXCL10 mRNA relative to a control DNA molecule; or
(ii) a reduction of a measure of inflammatory cytokine signaling relative to a control DNA molecule, e.g., at least a 2- or at least a 3-fold reduction, wherein the measure of inflammatory cytokine signaling is the average fold-change of IL6 mRNA and TNFα mRNA relative to a control DNA molecule,
wherein the control DNA molecule comprises the same sequence, same strandedness, and same circular or linear character as the dsDNA molecule, but comprises unmodified cytosine nucleotides in place of the chemically modified cytosine nucleotides.

63. The dsDNA molecule of any of the preceding embodiments, which encodes a protein, and which, when contacted to HepG2 cells, e.g., in an assay as described herein, results in expression at a level at least 50%, at least 60%, at least 70%, or at least 75% of the expression of a control DNA, wherein the control DNA molecule comprises the same sequence, same strandedness, and same circular or linear character as the dsDNA molecule, but comprises unmodified cytosine nucleotides in place of the chemically modified cytosine nucleotides.

64. The dsDNA molecule of any of the preceding embodiments, which comprises a therapeutic payload sequence, which, when contacted to HepG2 cells, results in expression of the therapeutic payload sequence at a level at least 50%, at least 60%, at least 70%, or at least 75% of the expression of the therapeutic payload sequence of a control DNA, wherein the control DNA molecule comprises the same sequence, same strandedness, and same circular or linear character as the dsDNA molecule, but comprises unmodified cytosine nucleotides in place of the chemically modified cytosine nucleotides.

65. The dsDNA molecule of any of the preceding embodiments, which encodes a protein, and which, when contacted to HEKa cells, e.g., in an assay as described herein, results in expression at a level at least 20%, at least 30%, at least 40%, at least 50%, or at least 55% of the expression of a control DNA, wherein the control DNA molecule comprises the same sequence, same strandedness, and same circular or linear character as the dsDNA molecule, but comprises unmodified cytosine nucleotides in place of the chemically modified cytosine nucleotides.

66. The dsDNA molecule of any of the preceding embodiments, which comprises a therapeutic payload sequence, which, when contacted to HEKa cells, results in expression of the therapeutic payload sequence at a level at least 20%, at least 30%, at least 40%, at least 50%, or at least 55% of the expression of the therapeutic payload sequence of a control DNA, wherein the control DNA molecule comprises the same sequence, same strandedness, and same circular or linear character as the dsDNA molecule, but comprises unmodified cytosine nucleotides in place of the chemically modified cytosine nucleotides.

67. The dsDNA molecule of any of the preceding embodiments, wherein the dsDNA molecule is a TDSC.

68. The dsDNA molecule of embodiment 67, wherein the TDSC comprises:
a) an upstream exonuclease-resistant DNA end form;
b) a double stranded region; and
c) a downstream exonuclease-resistant DNA end form.

69. The dsDNA molecule of embodiment 68, wherein one or both of the upstream exonuclease-resistant DNA end form and downstream exonuclease-resistant DNA end form are open ends.

70. The dsDNA molecule of embodiment 67, wherein the TDSC comprises:
a) an upstream DNA end form (e.g., an upstream exonuclease-resistant DNA end form) comprising a Y-adaptor configuration;
b) a double stranded region; and
c) a downstream DNA end form (e.g., a downstream exonuclease-resistant DNA end form) comprising a Y-adaptor configuration.

71. The dsDNA molecule of embodiment 68 or 69, wherein one or both of the upstream exonuclease-resistant DNA end form and downstream exonuclease-resistant DNA end form are blunt ends or sticky ends.

72. The dsDNA molecule of embodiment 67, wherein the TDSC comprises:
a) an upstream double stranded, blunt-ended DNA end form (e.g., an upstream exonuclease-resistant DNA end form that is double stranded and blunt-ended) comprising a phosphorothioate modification on each strand;
b) a double stranded region; and
c) a downstream double stranded, blunt-ended DNA end form (e.g., a downstream exonuclease-resistant DNA end form that is double stranded and blunt-ended) comprising a phosphorothioate modification on each strand.

73. The dsDNA molecule of embodiment 68, wherein one or both of the upstream exonuclease-resistant DNA end form and downstream exonuclease-resistant DNA end form are closed ends.

74. The dsDNA molecule of any of embodiments 68, 69, or 71-73, wherein one or both of the upstream exonuclease-resistant DNA end form and downstream exonuclease-resistant DNA end form comprise a loop.

75. The dsDNA molecule of embodiment 67, wherein the TDSC comprises:
a) an upstream DNA end form (e.g., an upstream exonuclease-resistant DNA end form) which is a closed end;
b) a double stranded region;
c) a downstream DNA end form (e.g., a downstream exonuclease-resistant DNA end form) which is a closed end.

76. The dsDNA molecule of any of embodiments 67-75, wherein the upstream DNA end form (e.g., upstream exonuclease-resistant DNA end form) comprises one or more chemically modified nucleotides.

77. The dsDNA molecule of any of embodiments 67-76, wherein the downstream DNA end form (e.g., downstream exonuclease-resistant DNA end form) comprises one or more chemically modified nucleotides.

78. The dsDNA molecule of any of embodiments 67-77, wherein the dsDNA molecule further comprises one or more chemically modified nucleotide that comprises a modification in the backbone, sugar, or nucleobase.

79. The dsDNA molecule of any of the preceding embodiments, wherein one or more of the chemically modified nucleotides is conjugated to a peptide or protein.

80. The dsDNA molecule of any of the preceding embodiments, wherein one or more of the chemically modified nucleotides comprises a phosphorothioate bond.

81. The dsDNA molecule of any of the preceding embodiments, wherein each of the first and second strands of the dsDNA molecule comprises one or more chemically modified nucleotides.

82. The dsDNA molecule of any of the preceding embodiments, wherein each of the first and second strands of the dsDNA molecule comprises one or more phosphorothioate bonds.

83. The dsDNA molecule of any of embodiments 68-82, wherein the upstream exonuclease-resistant DNA end form comprises at least 1, 2, 3, 4, 5, 6, 7, 8, 9, or 10 phosphorothioate bonds (e.g., between the 1, 2, 3, 4, 5, 6, 7, 8, 9, or 10 terminal nucleotides of the upstream exonuclease-resistant DNA end form, e.g., on the first strand, the second strand, or both of the first and second strands).

84. The dsDNA molecule of any of embodiments 68-83, wherein the upstream exonuclease-resistant DNA end form comprises at least 3 phosphorothioate bonds (e.g., between the 1, 2, 3, 4, 5, 6, 7, 8, 9, or 10 terminal nucleotides of the upstream exonuclease-resistant DNA end form, e.g., on the first strand, the second strand, or both of the first and second strands).

85. The dsDNA molecule of any of embodiments 68-84, wherein the upstream exonuclease-resistant DNA end form comprises at least 6 phosphorothioate bonds (e.g., between the 1, 2, 3, 4, 5, 6, 7, 8, 9, or 10 terminal nucleotides of the upstream exonuclease-resistant DNA end form, e.g., on the first strand, the second strand, or both of the first and second strands).

86. The dsDNA molecule of any of embodiments 68-85, wherein the downstream exonuclease-resistant DNA end form comprises at least 1, 2, 3, 4, 5, 6, 7, 8, 9, or 10 phosphorothioate bonds (e.g., between the 1, 2, 3, 4, 5, 6, 7, 8, 9, or 10 terminal nucleotides of the downstream exonuclease-resistant DNA end form, e.g., on the first strand, the second strand, or both of the first and second strands).

87. The dsDNA molecule of any of embodiments 68-86, wherein the downstream exonuclease-resistant DNA end form comprises at least 3 phosphorothioate bonds (e.g., between the 1, 2, 3, 4, 5, 6, 7, 8, 9, or 10 terminal nucleotides of the downstream exonuclease-resistant DNA end form, e.g., on the first strand, the second strand, or both of the first and second strands).

88. The dsDNA molecule of any of embodiments 68-87, wherein the downstream exonuclease-resistant DNA end form comprises at least 6 phosphorothioate bonds (e.g., between the 1, 2, 3, 4, 5, 6, 7, 8, 9, or 10 terminal nucleotides of the downstream exonuclease-resistant DNA end form, e.g., on the first strand, the second strand, or both of the first and second strands).

89. The dsDNA molecule of any of embodiments 68-88, wherein the upstream and downstream exonuclease-resistant DNA end form each comprises at least 1, 2, 3, 4, 5, 6, 7, 8, 9, or 10 phosphorothioate bonds (e.g., between the 1, 2, 3, 4, 5, 6, 7, 8, 9, or 10 terminal nucleotides of the upstream and downstream exonuclease-resistant DNA end forms, e.g., on the first strand, the second strand, or both of the first and second strands).

90. The dsDNA molecule of any of embodiments 68-89, wherein the upstream and downstream exonuclease-resistant DNA end form each comprises at least 3 phosphorothioate bonds (e.g., between the 1, 2, 3, 4, 5, 6, 7, 8, 9, or 10 terminal nucleotides of the upstream and downstream exonuclease-resistant DNA end forms, e.g., on the first strand, the second strand, or both of the first and second strands).

91. The dsDNA molecule of any of embodiments 68-90, wherein the upstream and downstream exonuclease-resistant DNA end form each comprises at least 6 phosphorothioate bonds (e.g., between the 1, 2, 3, 4, 5, 6, 7, 8, 9, or 10 terminal nucleotides of the upstream and downstream exonuclease-resistant DNA end forms, e.g., on the first strand, the second strand, or both of the first and second strands).

92. The dsDNA molecule of any of the preceding embodiments, wherein one or more of the chemically modified nucleotides comprises a methyl group.

93. The dsDNA molecule of embodiment 67, wherein the TDSC comprises:
a) an upstream exonuclease-resistant DNA end form;
b) a double stranded region;
c) a downstream exonuclease-resistant DNA end form,
   wherein one or both of the upstream exonuclease-resistant DNA end form and the downstream exonuclease-resistant DNA end form comprises a Y-adaptor configuration.

94. The dsDNA molecule of embodiment 93, wherein every nucleotide in the Y-adaptor is a chemically modified nucleotide.

95. The dsDNA molecule of embodiment 67, wherein the TDSC comprises:
a) an upstream exonuclease-resistant DNA end form;
b) a double stranded region;
c) a downstream exonuclease-resistant DNA end form,
   wherein one or both of the upstream exonuclease-resistant DNA end form and the downstream exonuclease-resistant DNA end form comprises one or more of: a nuclear targeting sequence, a maintenance sequence, a sequence that binds an endogenous polypeptide in a target cell.

96. The dsDNA molecule of embodiment 67, wherein the TDSC comprises:
a) an upstream exonuclease-resistant DNA end form;
b) a double stranded region;
c) a downstream exonuclease-resistant DNA end form,
   wherein one or both of the upstream exonuclease-resistant DNA end form and the downstream exonuclease-resistant DNA end form have one or more of the following characteristics:
i) does not comprise the nucleic acid sequences TATCAGCACACAATTGCCCATTATACGC (SEQ ID NO: 55) and GCGTATAATGGGCAATTGTGTGCTGATA (SEQ ID NO: 56), or nucleic acid sequences having at least 75%, at least 80%, at least 85%, at least 90%, at least 95%, at least 96%, at least 97%, at least 98%, or at least 99% sequence identity thereto; and/or the nucleic acid sequences TATCAGCACACAATAGTCCATTATACGC (SEQ ID NO: 57) and GCGTATAATGGACTATTGTGTGCTGATA (SEQ ID NO: 58);
ii) every nucleotide in the TDSC binds another nucleotide in the TDSC;
iii) the upstream exonuclease-resistant DNA end form has a loop size of less than about 28 or 56 nucleotides in length or greater than about 28 or 56 nucleotides in length; or
iv) the downstream exonuclease-resistant DNA end form has a loop size of less than about 28 or 56 nucleotides in length or greater than about 28 or 56 nucleotides in length.

97. The dsDNA molecule of any of the preceding embodiments, which comprises one or more of:
i) a promoter sequence (wherein optionally the promoter sequence is in the double stranded region);
ii) a payload sequence (e.g., a therapeutic payload sequence) operably linked to the promoter sequence (wherein optionally the payload sequence is in the double stranded region);
iii) a heterologous functional sequence, e.g., a nuclear targeting sequence or a regulatory sequence;
iv) a maintenance sequence; and/or
v) an origin of replication.

98. The dsDNA molecule of embodiment 78, which comprises:
i, ii, and iii;
i, ii, and iv;
i, ii, and v;
i, ii, iii, and iv;
i, ii, iii, and v;
i, ii, iv, and v; or
i, ii, iii, iv, and v.

99. The dsDNA molecule of any of the preceding embodiments, wherein the dsDNA molecule comprises a nuclear targeting sequence comprising a CT3 sequence (e.g., a sequence of AATTCTCCTCCCCACCTTCCCCACCCTCCCCA (SEQ ID NO: 59)), or a nucleic acid sequence having at least 75%, at least 80%, at least 85%, at least 90%, at least 95%, at least 96%, at least 97%, at least 98%, or at least 99% sequence identity thereto.

100. The dsDNA molecule of any of the preceding embodiments, wherein the dsDNA molecule comprises a nuclear targeting sequence that binds to an hnRNPK protein (e.g., a human hnRNPK protein).

101. The dsDNA molecule of any of preceding embodiments, which comprises a payload sequence, wherein the payload sequence encodes a polypeptide (e.g., a protein).

102. The dsDNA molecule of any of embodiments 97-101, which comprises a payload sequence, wherein the payload sequence encodes a functional RNA (e.g., a miRNA, siRNA, or tRNA).

103. The dsDNA molecule of any of the preceding embodiments, which comprises a therapeutic payload sequence, wherein at least 1%, at least 5%, at least 10%, at least 15%, at least 20%, at least 25%, at least 50%, or at least 75% of cytosine positions in the therapeutic payload sequence comprise the chemically modified cytosine nucleotide.

104. The dsDNA molecule of any of the preceding embodiments, which comprises a therapeutic payload sequence, wherein 1%-75% (e.g., 1-5%, 5-10%, 10-15%, 15-20%, 20-25%, 25-30%, 30-35%, 35-40%, 40-45%, 45-50%, 50-55%, 55-60%, 60-65%, 65-70%, or 70-75%) of cytosine positions in the therapeutic payload sequence comprise the chemically modified cytosine nucleotide.

105. The dsDNA molecule of any of the preceding embodiments, which comprises a payload sequence, wherein the payload sequence is heterologous to a target cell.

106. The dsDNA molecule of any of the preceding embodiments, wherein the double stranded region comprises a sense strand and an antisense strand.

107. The dsDNA molecule of embodiment 106, wherein the antisense strand comprises one or more chemically modified nucleotides.

108. The dsDNA molecule of embodiment 106 or 107, wherein the sense strand does not comprise any chemically modified nucleotides.

109. The dsDNA molecule of embodiment 106 or 107, wherein the sense strand comprises one or more chemically modified nucleotides.

110. The dsDNA molecule of any of embodiments 68-109, wherein the upstream exonuclease-resistant DNA end form is resistant to endonuclease digestion.

111. The dsDNA molecule of any of embodiments 68-110, wherein the upstream exonuclease-resistant DNA end form is resistant to immune sensor recognition.

112. The dsDNA molecule of any of embodiments 68-111, wherein the downstream exonuclease-resistant DNA end form is resistant to endonuclease digestion.

113. The dsDNA molecule of any of embodiments 68-112, wherein the downstream exonuclease-resistant DNA end form is resistant to immune sensor recognition.

114. The dsDNA molecule of any of embodiments 68-113, wherein the double stranded region is resistant to endonuclease digestion.

115. The dsDNA molecule of any of embodiments 68-114, wherein the double stranded region is resistant to immune sensor recognition.

116. The dsDNA molecule of any of embodiments 68-115, wherein the upstream DNA end form and the downstream DNA end form have the same nucleotide sequence.

117. The dsDNA molecule of any of embodiments 68-115, wherein the upstream DNA end form and the downstream DNA end form have different nucleotide sequences.

118. The dsDNA molecule of any of embodiments 68-117, wherein the upstream exonuclease-resistant DNA end form and the downstream exonuclease-resistant DNA end form have the same structure.

119. The dsDNA molecule of any of embodiments 68-117, wherein the upstream exonuclease-resistant DNA end form and the downstream exonuclease-resistant DNA end form have different structures.

120. The dsDNA molecule of any of embodiments 68-119, wherein one or both of the upstream exonuclease-resistant DNA end form and downstream exonuclease-resistant DNA end form are open ends (e.g., blunt ends, sticky ends, or Y-adaptors).

121. The dsDNA molecule of any of embodiments 68-120, wherein one or both of the upstream exonuclease-resistant DNA end form and downstream exonuclease-resistant DNA end form are closed ends (e.g., hairpins).

122. The dsDNA molecule of any of embodiments 73-121, wherein the closed end comprises one or more (e.g., at least 1, 2, 3, 4, 5, 6, 7, 8, 9, 10, 15, 20, 25, 30, 40, or 50) nucleotides that are not hybridized (e.g., are not part of a double-stranded region).

123. The dsDNA molecule of any of embodiments 73-121, wherein the closed end does not comprise any nucleotides that are not hybridized (e.g., wherein all nucleotides of the closed end are hybridized to another nucleotide).

124. The dsDNA molecule of any of embodiments 68-123, wherein the upstream DNA end form, the downstream DNA end form, or both, comprise at least one chemically modified nucleotide.

125. The dsDNA molecule of any of embodiments 68-124, wherein both of the upstream DNA end form and the downstream DNA end form comprise at least one chemically modified nucleotide on the sense strand and at least one chemically modified nucleotide on the antisense strand.

126. The dsDNA molecule of any of embodiments 68-125, wherein both the upstream DNA end form and the downstream DNA end form comprise chemically modified nucleotides at every sense strand position and every antisense strand position.

127. The dsDNA molecule of any of embodiments 68-126, wherein the upstream DNA end form, the downstream DNA end form, or both, comprises an inverted terminal repeat (ITR).

128. The dsDNA molecule of any of embodiments 68-127, wherein the upstream DNA end form, the downstream DNA end form, or both, comprises a protelomerase sequence.

129. The dsDNA molecule of embodiment 128, wherein one or more of the protelomerase sequences comprise (e.g., in 5′-to-3′ order) the nucleic acid sequences TATCAGCACACAATTGCCCATTATACGC (SEQ ID NO: 55) and GCGTATAATGGGCAATTGTGTGCTGATA (SEQ ID NO: 56), or nucleic acid sequences having at least 75%, at least 80%, at least 85%, at least 90%, at least 95%, at least 96%, at least 97%, at least 98%, or at least 99% sequence identity thereto.

130. The dsDNA molecule of embodiment 128 or 129, wherein one or more of the protelomerase sequences comprise (e.g., in 5′-to-3′ order) the nucleic acid sequences TATCAGCACACAATAGTCCATTATACGC (SEQ ID NO: 57) and GCGTATAATGGACTATTGTGTGCTGATA (SEQ ID NO: 58), or nucleic acid sequences having at least 75%, at least 80%, at least 85%, at least 90%, at least 95%, at least 96%, at least 97%, at least 98%, or at least 99% sequence identity thereto.

131. The dsDNA molecule of embodiment 128, wherein one or more of the protelomerase sequences comprise (e.g., in 5′-to-3′ order) the nucleic acid sequences ACCTATTTCAGCATACTACGC (SEQ ID NO: 60) and GCGTAGTATGCTGAAATAGGT (SEQ ID NO: 61), or nucleic acid sequences having at least 75%, at least 80%, at least 85%, at least 90%, at least 95%, at least 96%, at least 97%, at least 98%, or at least 99% sequence identity thereto.

132. The dsDNA molecule of embodiment 128, wherein one or more of the protelomerase sequences comprise (e.g., in 5′-to-3′ order) the nucleic acid sequence CACACAATTGCCCATTATACGCGCGTATAATGGGCAATTGTGTG (SEQ ID NO: 62), or a nucleic acid sequence having at least 75%, at least 80%, at least 85%, at least 90%, at least 95%, at least 96%, at least 97%, at least 98%, or at least 99% sequence identity thereto.

133. The dsDNA molecule of embodiment 128, wherein one or more of the protelomerase sequences comprise (e.g., in 5′-to-3′ order) the nucleic acid sequences:

| | (i) |
|---|---|
| (SEQ ID NO: 63) | |
| | TAAATATAATTTAA |
| | and |
| (SEQ ID NO: 64) | |
| | TTAAATTATATTTA, |
| | (ii) |
| (SEQ ID NO: 65) | |
| | AATATATAATCTAA |
| | and |
| (SEQ ID NO: 66) | |
| | TTAGATTATATATT, |
| | (iii) |
| (SEQ ID NO: 67) | |
| | TATTTATTATCTTT |
| | and |
| (SEQ ID NO: 68) | |
| | AAAGATAATAAATA, |
| | (iv) |
| (SEQ ID NO: 69) | |
| | ATATAATTTTTAATTAGTATAGAATATGTTAA |
| | and |
| (SEQ ID NO: 70) | |
| | TTAACATACTCTATACTAATTAAAAATTATAT, |
| | (v) |
| (SEQ ID NO: 71) | |
| | TATAATTTGATATTAGTACAAATCCC |
| | and |
| (SEQ ID NO: 72) | |
| | GGGATTTGTACTAATATCAAATTATA, |
| | (vi) |
| (SEQ ID NO: 73) | |
| | ATATAATATTTATTTAGTACAAAGTTC |
| | and |
| (SEQ ID NO: 74) | |
| | GAACTTTGTACTAAATAAATATTATAT, |
| | (vii) |
| (SEQ ID NO: 75) | |
| | ATATAATTTTTTATTAGTATAGAGTAT |
| | and |
| (SEQ ID NO: 76) | |
| | ATACTCTATACTAATAAAAAATTATAT, |
| | or |
| | (viii) |
| (SEQ ID NO: 63) | |
| | TAAATATAATTTAA |
| | and |
| (SEQ ID NO: 64) | |
| | TTAAATTATATTTA; |

or nucleic acid sequences having at least 75%, at least 80%, at least 85%, at least 90%, at least 95%, at least 96%, at least 97%, at least 98%, or at least 99% sequence identity thereto.

134. The dsDNA molecule of embodiment 133, wherein one or more of the protelomerase sequences further comprise (e.g., in 5′-to-3′ order) the nucleic acid sequences:

| | (i) |
|---|---|
| (SEQ ID NO: 77) | |
| | TAGTATAAAAAACTGT |
| | and |
| (SEQ ID NO: 78) | |
| | ACAGTTTTTTATACTA, |
| | (ii) |
| (SEQ ID NO: 79) | |
| | TAGTATACAAAAGATT |
| | and |
| (SEQ ID NO: 80) | |
| | AATCTTTTGTATACTA, |
| | (iii) |
| (SEQ ID NO: 81) | |
| | TAGTATATATATCTCT |
| | and |
| (SEQ ID NO: 82) | |
| | AGAGATATATATACTA, |
| | or |
| | (iv) |
| (SEQ ID NO: 83) | |
| | TAGTATAAAAAAAATT |
| | and |
| (SEQ ID NO: 84) | |
| | AATTTTTTTTATACTA; |

or nucleic acid sequences having at least 75%, at least 80%, at least 85%, at least 90%, at least 95%, at least 96%, at least 97%, at least 98%, or at least 99% sequence identity thereto.

135. The dsDNA molecule of any of embodiments 128-134, wherein the protelomerase sequences are produced by TelN protelomerase, ResT protelomerase, Tel PY54 protelomerase, or TelK protelomerase digestion.

136. The dsDNA molecule of any of embodiments 128-134, wherein the protelomerase sequences are not produced by TelN protelomerase digestion.

137. The dsDNA molecule of any of embodiments 128-134 or 136, wherein the protelomerase sequences are not produced by Tel PY54 protelomerase digestion.

138. The dsDNA molecule of any of embodiments 128-134, 136, or 137, wherein the protelomerase sequences are not produced by TelK protelomerase digestion.

139. The dsDNA molecule of any of embodiments 128-134 or 136-138, wherein the protelomerase sequences are not produced by ResT protelomerase digestion.

140. The dsDNA molecule of any of embodiments 128-139, wherein the protelomerase sequences are about 28 or 56 nucleotides in length.

141. The dsDNA molecule of any of embodiments 128-140, wherein the protelomerase sequences are less than 28 (e.g., less than 15, 20, 25, 26, 27, or 28) nucleotides in length.

142. The dsDNA molecule of any of embodiments 128-141, wherein the protelomerase sequences are between about 28 (e.g., 25, 26, 27, 28, 29, 30, 31, 32, 33, 34, or 35) nucleotides and about 56 (e.g., 50, 51, 52, 53, 54, 55, 56, 57, 58, 59, or 60) nucleotides in length.

143. The dsDNA molecule of any of embodiments 128-140 or 142, wherein the protelomerase sequences are greater than about 56 (e.g., greater than 50, 51, 52, 53, 54, 55, 56, 57, 58, 59, 60, 65, 70, 75, 80, 90, or 100) nucleotides in length.

144. The dsDNA molecule of any of embodiments 68-143, wherein the upstream DNA end form, the downstream DNA end form, or both, comprises a Y-adaptor.

145. The dsDNA molecule of any of embodiments 128-135, wherein the protelomerase sequence is produced from a first protelomerase recognition sequence (PRS) and a second PRS that are recognized by a TelN protelomerase or ResT protelomerase.

146. The dsDNA molecule of any of embodiments 128-135, wherein the protelomerase sequence is produced from a first protelomerase recognition sequence (PRS) and a second PRS that are recognized by a Tel PY54 protelomerase or TelK protelomerase.

147. The dsDNA molecule of any of embodiments 68-146, wherein one or both of the upstream exonuclease-resistant DNA end form and the downstream exonuclease-resistant DNA end form comprises at least one chemically modified nucleotide (e.g., comprises a chemical modification on every sense strand nucleotide and every antisense strand nucleotide).

148. The dsDNA molecule of any of embodiments 68-147, wherein one or both of the upstream exonuclease-resistant DNA end form and the downstream exonuclease-resistant DNA end form comprises one or more chemically modified nucleotides (e.g., phosphorothioate modified nucleotides).

149. The dsDNA molecule of any of the preceding embodiments, wherein the double-stranded region comprises one or more chemically modified nucleotides.

150. The dsDNA molecule of any of the preceding embodiments, wherein the double-stranded region encodes a payload sequence, and wherein the antisense strand for the payload sequence comprises one or more chemically modified nucleotides.

151. The dsDNA molecule of any of the preceding embodiments, wherein the double-stranded region encodes a payload sequence, and wherein the sense strand for the payload sequence comprises one or more chemically modified nucleotides.

152. The dsDNA molecule of any of the preceding embodiments, wherein the dsDNA molecule encodes a sequence encoding an RNA (e.g., an mRNA, siRNA, or miRNA).

153. The dsDNA molecule of any of embodiments 1-151, wherein the dsDNA molecule does not comprise a sequence encoding an RNA.

154. The dsDNA molecule of any of the preceding embodiments, wherein the dsDNA molecule can be replicated (e.g., by a DNA polymerase native to a cell comprising the dsDNA molecule).

155. The dsDNA molecule of any of embodiments 1-153, wherein the dsDNA molecule cannot be replicated.

156. The dsDNA molecule of any of the preceding embodiments, wherein the dsDNA molecule is linear and can be circularized.

157. The dsDNA molecule of any of embodiments 1-155, wherein the dsDNA molecule is linear and cannot be circularized.

158. The dsDNA molecule of any of the preceding embodiments, wherein the dsDNA molecule or a portion thereof can be integrated into the genome.

159. The dsDNA molecule of any of embodiments 1-157, wherein the dsDNA molecule or a portion thereof cannot be integrated into the genome.

160. The dsDNA molecule of any of the preceding embodiments, wherein the dsDNA molecule can be concatemerized.

161. The dsDNA molecule of any of embodiments 1-160, wherein the dsDNA molecule cannot be concatemerized.

162. The dsDNA molecule of any of the preceding embodiments, wherein 1%-100% (e.g., 1-5%, 5-10%, 10-15%, 15-20%, 20-25%, 25-30%, 30-35%, 35-40%, 40-45%, 45-50%, 50%-55%, 55-60%, 60-65%, 65-70%, 70-75%, 75-80%, 80-85%, 85-90%, 90-95%, or 95-100%) of cytosine positions in the sense strand of the dsDNA molecule comprise the chemically modified cytosine nucleotide, wherein the chemically modified cytosine nucleotide comprises 5-hydroxycytosine.

163. The dsDNA molecule of any of the preceding embodiments, wherein at least 1%, at least 5%, at least 10%, at least 15%, at least 20%, at least 25%, at least 50%, at least 75%, or at least 90% of cytosine positions in the sense strand of the dsDNA molecule comprise the chemically modified cytosine nucleotide, wherein the chemically modified cytosine nucleotide comprises 5-hydroxycytosine.

164. The dsDNA molecule of any of the preceding embodiments, which encodes a protein, and which, when contacted to HEKa cells, e.g., in an assay as decribed herein, results in expression at a level at least 60%, at least 70%, at least 80%, at least 90%, at least 100%, or at least 125% of the expression of a control DNA, wherein the control DNA molecule comprises the same sequence, same strandedness, and same circular or linear character as the dsDNA molecule, but comprises unmodified cytosine nucleotides in place of the chemically modified cytosine nucleotides.

165. The dsDNA molecule of any of the preceding embodiments, which was not produced by nick translation.

166. The dsDNA molecule of any of the preceding embodiments, which was not produced in a microorganism.

167. The dsDNA molecule of any of the preceding embodiments, which was produced in a cell-free system.

168. The dsDNA molecule of any of the preceding embodiments, which was produced by PCR.

169. The dsDNA molecule of any of the preceding embodiments, which does not encode a viral protein.

170. The dsDNA molecule of any of the preceding embodiments, which encodes only mammalian, e.g., human, proteins.

171. A pharmaceutical composition comprising the dsDNA molecule of any of the preceding embodiments.

172. The pharmaceutical composition of embodiment 171, wherein the dsDNA molecule lacks a material portion of vector backbone, e.g., lacks a vector backbone, or does not comprise a non-human (e.g., bacterial) origin of replication.

173. The pharmaceutical composition of embodiment 171 or 172, wherein the dsDNA molecule is unencapsidated.

174. The pharmaceutical composition of any of embodiments 171-173, wherein the dsDNA molecule does not comprise a viral packaging signal.

175. The pharmaceutical composition of any of embodiments 171-174, wherein the dsDNA molecule does not comprise a viral ITR.

176. The pharmaceutical composition of any of embodiments 171-175, which is essentially free of viral proteins.

177. The pharmaceutical composition of any of embodiments 171-176, which is essentially free of RNA.

178. The pharmaceutical composition of any of embodiments 171-177, which is essentially free of single stranded DNA.

179. The pharmaceutical composition of any of embodiments 171-178, which is essentially free of DNA fragments.

180. The pharmaceutical composition of any of embodiments 171-179, which is essentially free of open-ended double stranded DNA.

181. The pharmaceutical composition of any of embodiments 171-180, which is essentially free of microorganisms.

182. The pharmaceutical composition of any of embodiments 171-181, which is is essentially free of bacterial proteins.

183. The pharmaceutical composition of any of embodiments 171-182, which is essentially free of bacterial DNA.

184. The pharmaceutical composition of any of embodiments 171-183, wherein the pharmaceutical composition comprises a plurality of the dsDNA molecules, and wherein all dsDNA molecules in the pharmaceutical composition have substantially the same length in nucleotides (e.g., all dsDNA molecules in the pharmaceutical composition have the same length in nucleotides).

185. The pharmaceutical composition of any of embodiments 171-183, wherein the pharmaceutical composition comprises a plurality of the dsDNA molecules, and wherein at least 50%, at least 60%, at least 70%, at least 80%, or at least 90% of dsDNA molecules in the pharmaceutical composition have the same length in nucleotides.

186. The pharmaceutical composition of any of embodiments 171-185, wherein the pharmaceutical composition comprises a plurality of the dsDNA molecules, wherein the dsDNA molecules comprise a therapeutic payload sequence, and wherein the therapeutic payload sequences of dsDNA molecules in the pharmaceutical composition have substantially the same length in nucleotides (e.g., the therapeutic payload sequences of dsDNA molecules in the pharmaceutical composition have the same length in nucleotides).

187. The pharmaceutical composition of any of embodiments 171-186, wherein the pharmaceutical composition comprises a plurality of the dsDNA molecules, and wherein all dsDNA molecules in the pharmaceutical composition have a length of between 100, 200, 500, or 1000 nucleotides of each other.

188. The pharmaceutical composition of any of embodiments 171-187, wherein the pharmaceutical composition comprises a plurality of the dsDNA molecules, and wherein all dsDNA molecules in the pharmaceutical composition have a length of between 500-1000, 1000-2000, 2000-3000, 3000-4000, 4000-5000, 5000-6000, 6000-7000, 7000-8000, 8000-9000, 9000-10000, 10000-11000, or 11000-12000 nucleotides.

189. The pharmaceutical composition of any of embodiments 171-188, wherein the pharmaceutical composition comprises a plurality of the dsDNA molecules, and wherein all dsDNA molecules in the pharmaceutical composition encode substantially the same effector (e.g., all dsDNA molecules in the pharmaceutical composition encode the same effector).

190. The pharmaceutical composition of any of embodiments 171-189, wherein the pharmaceutical composition comprises a plurality of the dsDNA molecules, and wherein all dsDNA molecules in the pharmaceutical composition have substantially the same sequence (e.g., all dsDNA molecules in the pharmaceutical composition have the same sequence).

191. The pharmaceutical composition of any of embodiments 171-190, wherein the dsDNA molecule is comprised in a lipid nanoparticle (LNP).

192. The pharmaceutical composition of any of embodiments 171-191, further comprising an electroporation buffer.

193. The pharmaceutical composition of any of embodiments 171-192, further comprising a transfection reagent.

194. A pharmaceutical composition comprising a plurality of dsDNA molecules according to any of embodiments 1-170.

195. The pharmaceutical composition of embodiment 194, wherein the plurality comprises:
a) a first sub-population of dsDNA molecules according to any of embodiments 1-170, wherein all the dsDNA molecules in the first sub-population have the same DNA sequence, and
b) at least one additional dsDNA molecule according to any of embodiments 1-170, wherein the additional dsDNA molecule has a different DNA sequence from the dsDNA molecules in the first sub-population.

196. The pharmaceutical composition of embodiment 195, wherein the first sub-population of dsDNA molecules has a desired DNA sequence.

197. The pharmaceutical composition of embodiment 196, wherein the additional dsDNA molecule has one or more errors relative to the desired DNA sequence.

198. The pharmaceutical composition of embodiment 197, wherein the one or more errors comprises one or more of a substitution, an insertion, or a deletion.

199. The pharmaceutical composition of any of embodiments 195-198, wherein at least 20% or at least 30% of dsDNA molecules in the pharmaceutical composition are part of the first sub-population.

200. The pharmaceutical composition of any of embodiments 195-199, wherein 10%-15%, 15%-20%, 20%-25%, or 25%-30% of dsDNA molecules in the pharmaceutical composition are part of the first sub-population.

201. The pharmaceutical composition of any of embodiments 194-200, wherein the dsDNA molecules of the plurality comprise an amplicon region beginning at the start codon for the encoded polypeptide of the dsDNA molecules and extending at least 200 base pairs, at least 210 base pairs, at least 220 base pairs, at least 230 base pairs, at least 240 base pairs, or at least 250 base pairs in the direction of transcription.

202. The pharmaceutical composition of any of embodiments 194-201, wherein the dsDNA molecules of the plurality comprise an amplicon region beginning at the start codon for the encoded polypeptide of the dsDNA molecules and extending 200 base pairs to 210 base pairs, 210 base pairs to 220 base pairs, 220 base pairs to 230 base pairs, 230 base pairs to 240 base pairs, or 240 base pairs to 250 base pairs in the direction of transcription.

203. The pharmaceutical composition of any of embodiments 194-202, wherein the dsDNA molecules of the plurality comprise an amplicon region beginning at the start codon for the encoded polypeptide of the dsDNA molecules and extending 230 base pairs in the direction of transcription.

204. The pharmaceutical composition of any of embodiments 201-203, wherein the plurality comprises:
a) a first sub-population of dsDNA molecules according to any of embodiments 1-170, wherein each amplicon region in the first sub-population has the same DNA sequence, and
b) at least one additional dsDNA molecule according to any of embodiments 1-170, wherein the amplicon region of the additional dsDNA molecule has a different DNA sequence from the amplicon region in the first sub-population.

205. The pharmaceutical composition of embodiment 204, wherein the amplicon region of the first sub-population of dsDNA molecules has a desired DNA sequence.

206. The pharmaceutical composition of embodiment 205, wherein the amplicon region of the additional dsDNA molecule has one or more errors relative to the desired DNA sequence.

207. The pharmaceutical composition of embodiment 206, wherein the one or more errors comprises one or more of a substitution, an insertion, or a deletion.

208. The pharmaceutical composition of any of embodiments 204-207, wherein at least 30%, at least 40%, at least 50%, at least 60%, at least 70%, or at least 75% of dsDNA molecules in the pharmaceutical composition are part of the first sub-population.

209. The pharmaceutical composition of any of embodiments 204-208, wherein 30%-75%, e.g., 30%-40%, 40%-50%, 50%-60%, 60%-65%, 65%-70%, or 70%-75%, of dsDNA molecules in the pharmaceutical composition are part of the first sub-population.

210. The pharmaceutical composition of any of embodiments 196-209, wherein the dsDNA molecules in the pharmaceutical composition have an average of less than 5, less than 3, less than 2, less than 1.5, or less than 1.13 substitutions per kilobase relative to the desired DNA sequence.

211. The pharmaceutical composition of any of embodiments 196-210, wherein the dsDNA molecules in the pharmaceutical composition have an average of 1-5, e.g., 1-2, 2-3, 3-4, 4-5, or 1.13-2, or 1.13-3, substitutions per kilobase relative to the desired DNA sequence.

212. The pharmaceutical composition of any of embodiments 196-211, wherein the dsDNA molecules in the pharmaceutical composition have an average of less than 0.1, less than 0.05, less than 0.04, or less than 0.03 insertions per kilobase relative to the desired DNA sequence.

213. The pharmaceutical composition of any of embodiments 196-212, wherein the dsDNA molecules in the pharmaceutical composition have an average of 0.01-0.1, e.g., 0.01-0.05, 0.05-0.1, or 0.02-0.04, insertions per kilobase relative to the desired DNA sequence.

214. The pharmaceutical composition of any of embodiments 196-213, wherein the dsDNA molecules in the pharmaceutical composition have an average of less than 0.5, less than 0.25, less than 0.2, or less than 0.17 deletions per kilobase relative to the desired DNA sequence.

215. The pharmaceutical composition of any of embodiments 196-214, wherein the dsDNA molecules in the pharmaceutical composition have an average of 0.1-0.5, e.g., 0.1-0.3, 0.3-0.5, 0.15-0.25, or 0.1-0.2, deletions per kilobase relative to the desired DNA sequence.

216. The pharmaceutical composition of any of embodiments 196-215, wherein the dsDNA molecules in the pharmaceutical composition have an average of less than 4, less than 3, less than 2, or less than 1.33 errors per kilobase relative to the desired DNA sequence.

217. The pharmaceutical composition of any of embodiments 196-216, wherein the dsDNA molecules in the pharmaceutical composition have an average of 1-4, e.g., 1-2, 2-3, 3-4, or 1.33-3, errors per kilobase relative to the desired DNA sequence.

218. The pharmaceutical composition of any of embodiments 205-217, wherein in an amplicon region, on average at least 98%, at least 99%, at least 99.5%, at least 99.8%, or at least 99.845% of the positions that are adenine in the desired DNA sequence are adenine in the dsDNA molecules in the pharmaceutical composition.

219. The pharmaceutical composition of any of embodiments 205-218, wherein in an amplicon region, on average 98%-99.85%, e.g., 98%-99%, 99%-99.5%, 99.5%-99.85%, or 99%-99.85%, of the positions that are adenine in the desired DNA sequence are adenine in the dsDNA molecules in the pharmaceutical composition.

220. The pharmaceutical composition of any of embodiments 205-219, wherein in an amplicon region, on average less than 0.05%, less than 0.03%, less than 0.02%, or less than 0.017% of the positions that are adenine in the desired DNA sequence are cytosine in the dsDNA molecules in the pharmaceutical composition.

221. The pharmaceutical composition of any of embodiments 205-220, wherein in an amplicon region, on average 0.015%-0.05%, e.g., 0.015%-0.03%, 0.03%-0.05%, or 0.015%-0.02%, of the positions that are adenine in the desired DNA sequence are cytosine in the dsDNA molecules in the pharmaceutical composition.

222. The pharmaceutical composition of any of embodiments 205-221, wherein in an amplicon region, on average less than 0.5%, less than 0.3%, less than 0.2%, less than 0.15%, or less than 0.102% of the positions that are adenine in the desired DNA sequence are guanine in the dsDNA molecules in the pharmaceutical composition.

223. The pharmaceutical composition of any of embodiments 205-222, wherein in an amplicon region, on average 0.1%-0.5%, e.g., 0.1%-0.3%, 0.3%-0.5%, 0.1%-0.2%, or 0.1-0.15%, of the positions that are adenine in the desired DNA sequence are guanine in the dsDNA molecules in the pharmaceutical composition.

224. The pharmaceutical composition of any of embodiments 205-223, wherein in an amplicon region, on average less than 0.1%, less than 0.05%, less than 0.03%, less than 0.025%, or less than 0.022% of the positions that are adenine in the desired DNA sequence are thymine in the dsDNA molecules in the pharmaceutical composition.

225. The pharmaceutical composition of any of embodiments 205-224, wherein in an amplicon region, on average 0.02%-0.1%, e.g., 0.02%-0.05%, 0.05%-0.1%, 0.02-0.03%, or 0.02-0.025%, of the positions that are adenine in the desired DNA sequence are thymine in the dsDNA molecules in the pharmaceutical composition.

226. The pharmaceutical composition of any of embodiments 205-225, wherein in an amplicon region, on average less than 0.1%, less than 0.05%, less than 0.03%, less than 0.02%, less than 0.015%, or less than 0.013% of the positions that are adenine in the desired DNA sequence are deleted in the dsDNA molecules in the pharmaceutical composition.

227. The pharmaceutical composition of any of embodiments 205-226, wherein in an amplicon region, on average 0.01%-0.1%, e.g., 0.01%-0.05%, 0.05%-0.1%, 0.01%-0.03%, 0.01%-0.02%, or 0.01-0.015%, of the positions that are adenine in the desired DNA sequence are deleted in the dsDNA molecules in the pharmaceutical composition.

228. The pharmaceutical composition of any of embodiments 205-227, wherein in an amplicon region, on average less than 0.01%, less than 0.005%, less than 0.003%, less than 0.0025%, or less than 0.002% of the positions that are adenine in the desired DNA sequence comprise one or more inserted nucleotides 5′ or 3′ of the adenine in the dsDNA molecules in the pharmaceutical composition.

229. The pharmaceutical composition of any of embodiments 205-228, wherein in an amplicon region, on average 0.0015%-0.01%, e.g., 0.0015%-0.005%, 0.005%-0.01%, 0.0015%-0.003%, or 0.0015%-0.0025%, of the positions that are adenine in the desired DNA sequence comprise one or more inserted nucleotides 5′ or 3′ of the adenine in the dsDNA molecules in the pharmaceutical composition.

230. The pharmaceutical composition of any of embodiments 205-229, wherein in an amplicon region, on average at least 98%, at least 99%, at least 99.5%, at least 99.8%, or at least 99.874% of the positions that are cytosine in the desired DNA sequence are cytosine in the dsDNA molecules in the pharmaceutical composition.

231. The pharmaceutical composition of any of embodiments 205-230, wherein in an amplicon region, on average 98%-99.88%, e.g., 98%-99%, 99%-99.88%, 99.5%-99.88%, or 99.8%-99.88%, of the positions that are cytosine in the desired DNA sequence are cytosine in the dsDNA molecules in the pharmaceutical composition.

232. The pharmaceutical composition of any of embodiments 205-231, wherein in an amplicon region, on average less than 0.1%, less than 0.05%, less than 0.03%, or less than 0.029% of the positions that are cytosine in the desired DNA sequence are adenine in the dsDNA molecules in the pharmaceutical composition.

233. The pharmaceutical composition of any of embodiments 205-232, wherein in an amplicon region, on average 0.025%-0.1%, e.g., 0.025%-0.05%, 0.05%-0.1%, or 0.025%-0.03%, of the positions that are cytosine in the desired DNA sequence are adenine in the dsDNA molecules in the pharmaceutical composition.

234. The pharmaceutical composition of any of embodiments 205-233, wherein in an amplicon region, on average less than 0.02%, less than 0.015%, less than 0.01%, less than 0.009%, or less than 0.008% of the positions that are cytosine in the desired DNA sequence are guanine in the dsDNA molecules in the pharmaceutical composition.

235. The pharmaceutical composition of any of embodiments 205-234, wherein in an amplicon region, on average 0.005%-0.02%, e.g., 0.005%-0.015%, 0.015%-0.02%, 0.005%-0.01%, or 0.005%-0.009%, of the positions that are cytosine in the desired DNA sequence are guanine in the dsDNA molecules in the pharmaceutical composition.

236. The pharmaceutical composition of any of embodiments 205-235, wherein in an amplicon region, on average less than 0.1%, less than 0.08%, less than 0.07%, or less than 0.066% of the positions that are cytosine in the desired DNA sequence are thymine in the dsDNA molecules in the pharmaceutical composition.

237. The pharmaceutical composition of any of embodiments 205-236, wherein in an amplicon region, on average 0.06%-0.1%, e.g., 0.06%-0.08%, 0.08%-0.1%, or 0.06%-0.07%, of the positions that are cytosine in the desired DNA sequence are thymine in the dsDNA molecules in the pharmaceutical composition.

238. The pharmaceutical composition of any of embodiments 205-237, wherein in an amplicon region, on average less than 0.1%, less than 0.05%, less than 0.03%, less than 0.025%, or less than 0.02% of the positions that are cytosine in the desired DNA sequence are deleted in the dsDNA molecules in the pharmaceutical composition.

239. The pharmaceutical composition of any of embodiments 205-238, wherein in an amplicon region, on average 0.01%-0.1%, e.g., 0.01%-0.05%, 0.05%-0.1%, 0.01%-0.03%, or 0.01%-0.025%, of the positions that are cytosine in the desired DNA sequence are deleted in the dsDNA molecules in the pharmaceutical composition.

240. The pharmaceutical composition of any of embodiments 205-239, wherein in an amplicon region, on average less than 0.01%, less than 0.005%, less than 0.004%, less than 0.0035%, or less than 0.003% of the positions that are cytosine in the desired DNA sequence comprise one or more inserted nucleotides 5′ or 3′ of the cytosine in the dsDNA molecules in the pharmaceutical composition.

241. The pharmaceutical composition of any of embodiments 205-240, wherein in an amplicon region, on average 0.0025%-0.01%, e.g., 0.0025%-0.005%, 0.005%-0.01%, 0.0025%-0.004%, or 0.0025%-0.0035%, of the positions that are cytosine in the desired DNA sequence comprise one or more inserted nucleotides 5′ or 3′ of the cytosine in the dsDNA molecules in the pharmaceutical composition.

242. The pharmaceutical composition of any of embodiments 205-241, wherein in an amplicon region, on average at least 98%, at least 99%, at least 99.5%, at least 99.8%, or at least 99.875% of the positions that are guanine in the desired DNA sequence are guanine in the dsDNA molecules in the pharmaceutical composition.

243. The pharmaceutical composition of any of embodiments 205-242, wherein in an amplicon region, on average 98%-99.88%, e.g., 98%-99%, 99%-99.88%, 99.5%-99.88%, or 99.8%-99.88%, of the positions that are guanine in the desired DNA sequence are guanine in the dsDNA molecules in the pharmaceutical composition.

244. The pharmaceutical composition of any of embodiments 205-243, wherein in an amplicon region, on average less than 0.1%, less than 0.08%, less than 0.07%, or less than 0.065% of the positions that are guanine in the desired DNA sequence are adenine in the dsDNA molecules in the pharmaceutical composition.

245. The pharmaceutical composition of any of embodiments 205-244, wherein in an amplicon region, on average 0.06%-0.1%, e.g., 0.06%-0.08%, 0.08%-0.1%, or 0.06%-0.07%, of the positions that are guanine in the desired DNA sequence are adenine in the dsDNA molecules in the pharmaceutical composition.

246. The pharmaceutical composition of any of embodiments 205-245, wherein in an amplicon region, on average less than 0.1%, less than 0.05%, less than 0.03%, less than 0.02%, or less than 0.01% of the positions that are guanine in the desired DNA sequence are cytosine in the dsDNA molecules in the pharmaceutical composition.

247. The pharmaceutical composition of any of embodiments 205-246, wherein in an amplicon region, on average 0.009%-0.1%, e.g., 0.009%-0.05%, 0.05%-0.1%, 0.009%-0.03%, or 0.009%-0.02%, of the positions that are guanine in the desired DNA sequence are cytosine in the dsDNA molecules in the pharmaceutical composition.

248. The pharmaceutical composition of any of embodiments 205-247, wherein in an amplicon region, on average less than 0.1%, less than 0.05%, less than 0.03%, less than 0.025%, or less than 0.024% of the positions that are guanine in the desired DNA sequence are thymine in the dsDNA molecules in the pharmaceutical composition.

249. The pharmaceutical composition of any of embodiments 205-248, wherein in an amplicon region, on average 0.02%-0.1%, e.g., 0.02%-0.05%, 0.05%-0.1%, 0.02%-0.03%, or 0.02%-0.025%, of the positions that are guanine in the desired DNA sequence are thymine in the dsDNA molecules in the pharmaceutical composition.

250. The pharmaceutical composition of any of embodiments 205-249, wherein in an amplicon region, on average less than 0.1%, less than 0.05%, less than 0.03%, less than 0.025%, or less than 0.021% of the positions that are guanine in the desired DNA sequence are deleted in the dsDNA molecules in the pharmaceutical composition.

251. The pharmaceutical composition of any of embodiments 205-250, wherein in an amplicon region, on average 0.02%-0.1%, e.g., 0.02%-0.05%, 0.05%-0.1%, 0.02%-0.03%, or 0.02%-0.025%, of the positions that are guanine in the desired DNA sequence are deleted in the dsDNA molecules in the pharmaceutical composition.

252. The pharmaceutical composition of any of embodiments 205-251, wherein in an amplicon region, on average less than 0.01%, less than 0.007%, less than 0.006%, less than 0.005%, or less than 0.004% of the positions that are guanine in the desired DNA sequence comprise one or more inserted nucleotides 5′ or 3′ of the guanine in the dsDNA molecules in the pharmaceutical composition.

253. The pharmaceutical composition of any of embodiments 205-252, wherein in an amplicon region, on average 0.0035%-0.01%, e.g., 0.0035%-0.007%, 0.007%-0.01%, 0.0035%-0.006%, or 0.0035%-0.005%, of the positions that are guanine in the desired DNA sequence comprise one or more inserted nucleotides 5′ or 3′ of the guanine in the dsDNA molecules in the pharmaceutical composition.

254. The pharmaceutical composition of any of embodiments 205-253, wherein in an amplicon region, on average at least 98%, at least 99%, at least 99.5%, at least 99.8%, or at least 99.865% of the positions that are thymine in the desired DNA sequence are thymine in the dsDNA molecules in the pharmaceutical composition.

255. The pharmaceutical composition of any of embodiments 205-254, wherein in an amplicon region, on average 98%-99.87%, e.g., 98%-99%, 99%-99.87%, 99.5%-99.87%, or 99.8%-99.87%, of the positions that are thymine in the desired DNA sequence are thymine in the dsDNA molecules in the pharmaceutical composition.

256. The pharmaceutical composition of any of embodiments 205-255, wherein in an amplicon region, on average less than 0.1%, less than 0.05%, less than 0.04%, less than 0.03%, or less than 0.025% of the positions that are thymine in the desired DNA sequence are adenine in the dsDNA molecules in the pharmaceutical composition.

257. The pharmaceutical composition of any of embodiments 205-256, wherein in an amplicon region, on average 0.02%-0.1%, e.g., 0.02%-0.05%, 0.05%-0.1%, 0.02%-0.04%, or 0.02%-0.03%, of the positions that are thymine in the desired DNA sequence are adenine in the dsDNA molecules in the pharmaceutical composition.

258. The pharmaceutical composition of any of embodiments 205-257, wherein in an amplicon region, on average less than 0.1%, less than 0.09%, less than 0.08%, less than 0.07%, or less than 0.069% of the positions that are thymine in the desired DNA sequence are cytosine in the dsDNA molecules in the pharmaceutical composition.

259. The pharmaceutical composition of any of embodiments 205-258, wherein in an amplicon region, on average 0.06%-0.1%, e.g., 0.06%-0.08%, 0.08%-0.1%, 0.06%-0.09%, or 0.06%-0.07%, of the positions that are thymine in the desired DNA sequence are cytosine in the dsDNA molecules in the pharmaceutical composition.

260. The pharmaceutical composition of any of embodiments 205-259, wherein in an amplicon region, on average less than 0.1%, less than 0.05%, less than 0.04%, less than 0.03%, or less than 0.028% of the positions that are thymine in the desired DNA sequence are guanine in the dsDNA molecules in the pharmaceutical composition.

261. The pharmaceutical composition of any of embodiments 205-260, wherein in an amplicon region, on average 0.02%-0.1%, e.g., 0.02%-0.05%, 0.05%-0.1%, 0.02%-0.04%, or 0.02%-0.03%, of the positions that are thymine in the desired DNA sequence are guanine in the dsDNA molecules in the pharmaceutical composition.

262. The pharmaceutical composition of any of embodiments 205-261, wherein in an amplicon region, on average less than 0.1%, less than 0.05%, less than 0.02%, less than 0.015%, or less than 0.011% of the positions that are thymine in the desired DNA sequence are deleted in the dsDNA molecules in the pharmaceutical composition.

263. The pharmaceutical composition of any of embodiments 205-262, wherein in an amplicon region, on average 0.01%-0.1%, e.g., 0.01%-0.05%, 0.05%-0.1%, 0.01-0.02%, or 0.01%-0.015%, of the positions that are thymine in the desired DNA sequence are deleted in the dsDNA molecules in the pharmaceutical composition.

264. The pharmaceutical composition of any of embodiments 205-263, wherein in an amplicon region, on average less than 0.01%, less than 0.005%, less than 0.003%, less than 0.0025%, or less than 0.002% of the positions that are thymine in the desired DNA sequence comprise one or more inserted nucleotides 5′ or 3′ of the thymine in the dsDNA molecules in the pharmaceutical composition.

265. The pharmaceutical composition of any of embodiments 205-264, wherein in an amplicon region, on average 0.0015%-0.01%, e.g., 0.0015%-0.005%, 0.005%-0.01%, 0.0015%-0.003%, or 0.0015%-0.0025%, of the positions that are thymine in the desired DNA sequence comprise one or more inserted nucleotides 5′ or 3′ of the thymine in the dsDNA molecules in the pharmaceutical composition.

266. The pharmaceutical composition of any of embodiments 194-265, wherein, when the plurality of dsDNA molecules is introduced into a cell, the cell transcribes the dsDNA molecules to produce a plurality of RNA molecules, the plurality of RNA molecules comprising:
a) a first sub-population of RNA molecules, wherein all the RNA molecules in the first sub-population have the same RNA sequence, and
b) at least one additional RNA molecule, wherein the additional RNA molecule has a different RNA sequence from the RNA molecules in the first sub-population.

267. The pharmaceutical composition of embodiment 266, wherein the first sub-population of RNA molecules has a desired RNA sequence.

268. The pharmaceutical composition of embodiment 267, wherein the additional RNA molecule has one or more errors relative to the desired RNA sequence.

269. The pharmaceutical composition of embodiment 268, wherein the one or more errors comprises one or more of a substitution, an insertion, or a deletion.

270. The pharmaceutical composition of any of embodiments 266-268, wherein at least 20% or at least 30% of RNA molecules in the plurality are part of the first sub-population.

271. The pharmaceutical composition of any of embodiments 266-269, wherein 10%-15%, 15%-20%, 20%-25%, or 25%-30% of RNA molecules in the pharmaceutical composition are part of the first sub-population.

272. The pharmaceutical composition of any of embodiments 194-271, wherein, when the plurality of dsDNA molecules is introduced into a cell, the cell transcribes the dsDNA molecules to produce a plurality of RNA molecules, the plurality of RNA molecules comprising: an amplicon region beginning at the start codon for the encoded polypeptide of the dsDNA molecules and extending at least 200 base pairs, at least 210 base pairs, at least 220 base pairs, at least 230 base pairs, at least 240 base pairs, or at least 250 base pairs in the direction of transcription.

273. The pharmaceutical composition of any of embodiments 194-272, when the plurality of dsDNA molecules is introduced into a cell, the cell transcribes the dsDNA molecules to produce a plurality of RNA molecules, the plurality of RNA molecules comprising: an amplicon region beginning at the start codon for the encoded polypeptide of the dsDNA molecules and extending 200 base pairs to 210 base pairs, 210 base pairs to 220 base pairs, 220 base pairs to 230 base pairs, 230 base pairs to 240 base pairs, or 240 base pairs to 250 base pairs in the direction of transcription.

274. The pharmaceutical composition of any of embodiments 194-273, when the plurality of dsDNA molecules is introduced into a cell, the cell transcribes the dsDNA molecules to produce a plurality of RNA molecules, the plurality of RNA molecules comprising: an amplicon region beginning at the start codon for the encoded polypeptide of the dsDNA molecules and extending 230 base pairs in the direction of transcription.

275. The pharmaceutical composition of any of embodiments 272-274, wherein the plurality of RNA molecules comprises:
a) a first sub-population of RNA molecules, wherein each amplicon region in the first sub-population has the same RNA sequence, and
b) at least one additional RNA molecule, wherein the amplicon region of the additional RNA molecule has a different RNA sequence from the amplicon region in the first sub-population.

276. The pharmaceutical composition of embodiment 275, wherein the amplicon region of the first sub-population of RNA molecules has a desired RNA sequence.

277. The pharmaceutical composition of embodiment 276, wherein the amplicon region of the additional RNA molecule has one or more errors relative to the desired RNA sequence.

278. The pharmaceutical composition of embodiment 277, wherein the one or more errors comprises one or more of a substitution, an insertion, or a deletion.

279. The pharmaceutical composition of any of embodiments 275-278, wherein at least 10%, at least 20%, at least 30%, at least 40%, at least 50%, at least 60%, at least 70%, at least 75%, or at least 80% of the RNA molecules are part of the first sub-population.

280. The pharmaceutical composition any of embodiments 275-279, wherein 10%-20%, 20%-30%, 30%-40%, 40%-50%, 50%-60%, 60%-70%, 70%-75%, or 75%-80% of the RNA molecules are part of the first sub-population.

281. The pharmaceutical composition of any of embodiments 267-280, wherein the RNA molecules in the plurality have an average of less than 5, less than 4, less than 3, less than 2, less than 1.5 or less than 1.1 substitutions per kilobase relative to the desired RNA sequence.

282. The pharmaceutical composition of any of embodiments 267-281, wherein the RNA molecules in the plurality have an average of 1-5, e.g., 1-3, 3-5, or 1.1-3, substitutions per kilobase relative to the desired RNA sequence.

283. The pharmaceutical composition of any of embodiments 267-282, wherein the RNA molecules in the plurality have an average of less than 0.5, less than 0.2, less than 0.1, less than 0.08, or less than 0.07 insertions per kilobase relative to the desired RNA sequence.

284. The pharmaceutical composition of any of embodiments 267-283, wherein the RNA molecules in the plurality have an average of 0.06-0.5, e.g., 0.06-0.1, 0.1-0.5, or 0.07-0.2, insertions per kilobase relative to the desired RNA sequence.

285. The pharmaceutical composition of any of embodiments 267-284, wherein the RNA molecules in the plurality have an average of less than 1, less than 0.5, less than 0.4, or less than 0.3 deletions per kilobase relative to the desired RNA sequence.

286. The pharmaceutical composition of any of embodiments 267-285, wherein the RNA molecules in the plurality have an average of 0.29-1, e.g., 0.29-0.5, 0.5-1, or 0.3-0.5, deletions per kilobase relative to the desired RNA sequence.

287. The pharmaceutical composition of any of embodiments 267-286, wherein the RNA molecules in the plurality have an average of less than 5, less than 3, less than 2, less than 1.5, or less than 1.47 errors per kilobase relative to the desired RNA sequence.

288. The pharmaceutical composition of any of embodiments 267-287, wherein the RNA molecules in the plurality have an average of 1.46-5, e.g., 1.46-2, 2-3, 3-5, or 1.46-3, errors per kilobase relative to the desired RNA sequence.

289. The pharmaceutical composition of any of embodiments 266-288, wherein the cell type is HEKa.

290. The pharmaceutical composition of any of embodiments 194-289, wherein at least 80% of the dsDNA molecules of the plurality have the chemically modified cytosine nucleotide in at least 1%, at least 5%, at least 10%, at least 15%, at least 20%, at least 25%, at least 50%, or at least 75% of cytosine positions in the dsDNA molecule.

291. The pharmaceutical composition of any of embodiments 194-289, wherein at least 80% of the dsDNA molecules of the plurality have the chemically modified cytosine nucleotide in 1%-75% (e.g., 1-5%, 5-10%, 10-15%, 15-20%, 20-25%, 25-30%, 30-35%, 35-40%, 40-45%, 45-50%, 50-55%, 55-60%, 60-65%, 65-70%, or 70-75%) of cytosine positions in the dsDNA molecule.

292. The pharmaceutical composition of any of embodiments 194-289, wherein at least 50% of the dsDNA molecules of the plurality have the chemically modified cytosine nucleotide in at least 1%, at least 5%, at least 10%, at least 15%, at least 20%, at least 25%, at least 50%, or at least 75% of cytosine positions in the dsDNA molecule.

293. The pharmaceutical composition of any of embodiments 194-289, wherein at least 50% of the dsDNA molecules of the plurality have the chemically modified cytosine nucleotide in 1%-75% (e.g., 1-5%, 5-10%, 10-15%, 15-20%, 20-25%, 25-30%, 30-35%, 35-40%, 40-45%, 45-50%, 50-55%, 55-60%, 60-65%, 65-70%, or 70-75%) of cytosine positions in the dsDNA molecule.

294. The pharmaceutical composition of any of embodiments 194-289, wherein at least 90% of the dsDNA molecules of the plurality have the chemically modified cytosine nucleotide in at least 1%, at least 5%, at least 10%, at least 15%, at least 20%, at least 25%, at least 50%, or at least 75% of cytosine positions in the dsDNA molecule.

295. The pharmaceutical composition of any of embodiments 194-289, wherein at least 90% of the dsDNA molecules of the plurality have the chemically modified cytosine nucleotide in 1%-75% (e.g., 1-5%, 5-10%, 10-15%, 15-20%, 20-25%, 25-30%, 30-35%, 35-40%, 40-45%, 45-50%, 50-55%, 55-60%, 60-65%, 65-70%, or 70-75%) of cytosine positions in the dsDNA molecule.

296. A method of making or manufacturing a double stranded DNA (dsDNA) molecule, the method comprising:
(a) providing a composition comprising a DNA template (e.g., a plasmid), a forward primer, a reverse primer, a DNA polymerase, unmodified deoxyribose nucleotides, and a chemically modified cytosine nucleotide having a substitution other than hydrogen at carbon 5 of the cytosine; and
(b) performing a polymerase chain reaction on the composition of (a), thereby making or manufacturing the dsDNA molecule, wherein optionally the dsDNA molecule is a dsDNA molecule of any of embodiments 1-170.

297. The method of embodiment 296, wherein the method further comprises purification of the dsDNA molecule, e.g., wherein purification comprises use of a DNA purification column or agarose gel purification.

298. The method of embodiment 296 or 297, wherein the DNA polymerase comprises a KOD polymerase, a KOD Xtreme polymerase, a Deep Vent polymerase, or KOD Multi & Epi polymerase.

299. The method of any of embodiments 296-298, wherein the unmodified deoxyribose nucleotides comprise dATP, dCTP, dTTP, and dGTP.

300. A method of making or manufacturing a double stranded DNA (dsDNA) molecule comprising a chemically modified cytosine nucleotide having a substitution other than hydrogen at carbon 5 of the cytosine, the method comprising:
(a) providing an input dsDNA comprising cytosine nucleotides, e.g., modified or unmodified cytosine nucleotides; and
(b) incubating the input dsDNA of (a) with an enzyme that chemically modifies cytosine, e.g., a 5-hmC glucosyltransferase,
   thereby making or manufacturing the dsDNA molecule comprising the chemically modified cytosine nucleotide, wherein optionally the dsDNA molecule comprises a dsDNA molecule of any of embodiments 1-170.

301. The method of embodiment 300, wherein (a) further comprises performing a polymerase chain reaction on a composition comprising a DNA template, e.g., a plasmid, a forward primer, a reverse primer, a DNA polymerase, and deoxyribose nucleotides, e.g., unmodified or modified deoxyribose nucleotides.

302. The method of embodiment 300 or 301, wherein the chemically modified cytosine nucleotide comprises glucosyl-5-hydroxymethylcytosine.

303. The method of any of embodiments 296-302, wherein the percentage of cytosine nucleotides that are chemically modified cytosine nucleotides in the composition of (a) is 10%-20%, 20%-30%, 30%-40%, 40%-50%, 50%-60%, 60%-70%, or 70%-80%.

304. The method of any of embodiments 296-299 or 301-303, wherein the forward primer, the reverse primer, or both contains a protelomerase recognition sequence, e.g., a TelN protelomerase recognition sequence.

305. The method of any of embodiments 296-304, wherein the method further comprises (e.g., after step (b)):
(c) incubating the dsDNA molecule with a protelomerase, e.g., a TelN protelomerase.

306. The method of any of embodiments 296-299 or 301-305, wherein the forward primer, the reverse primer, or both contains a restriction enzyme recognition sequence.

307. The method of embodiment 306, wherein the method further comprises:
(i) incubating the dsDNA molecule with a restriction enzyme that cleaves the restriction enzyme recognition sequence, thereby making a cleaved dsDNA molecule;
(ii) incubating the cleaved dsDNA molecule with a DNA ligase, e.g., a T3 DNA ligase, thereby making a ligated dsDNA molecule; and/or
(iii) optionally, incubating the ligated dsDNA molecule with an exonuclease, e.g., a T5 exonuclease.

308. The method of any of embodiments 296-307, the method further comprising:
(d) ligating:
   the dsDNA molecule to
   a hairpin DNA molecule comprising: a loop region and a double-stranded region comprising one or more chemically modified nucleotides.

309. The method of any of embodiments 296-308, the method further comprising ligating:
the dsDNA molecule to
a self-annealed DNA molecule comprising a first region and a second region, wherein the first region is hybridized to the second region.

310. The method of embodiment 309, wherein the self-annealed DNA molecule further comprises a loop between the first region and the second region.

311. The method of embodiment 310, wherein the loop comprises a heterologous functional sequence, e.g., a nuclear targeting sequence (e.g., a CT3 sequence); or a regulatory sequence.

312. The method of embodiment 309, wherein the self-annealed DNA molecule does not comprise any nucleotides that are not hybridized (e.g., wherein all nucleotides of the self-annealed DNA molecule are hybridized to another nucleotide).

313. The method of any of embodiments 308-312, which further comprises ligating a second hairpin DNA molecule to the dsDNA molecule, wherein the second hairpin DNA molecule comprises a loop region and a double-stranded region, wherein optionally the second hairpin DNA molecule comprises one or more chemically modified nucleotides in one or both of the loop region or the double stranded region.

314. A dsDNA molecule produced by the method of any of embodiments 296-313.

315. A method of making or manufacturing a TDSC, the method comprising:
a) providing the dsDNA molecule made by a method of any embodiments 296-313, wherein the dsDNA molecule comprises closed ends;
b) incubating the TDSC with a double stranded DNA exonuclease, e.g., Exonuclease III, e.g., e.g., 1 μL of Exonuclease III per 5 μg of DNA in 50 μL, for 1 hour at 37° C., e.g., as described in Example 2;
c) optionally, purifying the TDSC treated in step b), e.g., by Silica membrane column, e.g., as described in Example 2,
thereby making or manufacturing the TDSC.

316. A method of expressing a heterologous payload in a target cell, the method comprising:
(i) introducing into a target cell the dsDNA molecule of any of embodiments 1-170 or 314, wherein the dsDNA molecule encodes a heterologous payload; and
(ii) maintaining (e.g., incubating) the cell under conditions suitable for expressing the heterologous payload from the dsDNA molecule;
   thereby expressing the heterologous payload in the target cell.

317. A method of modulating (e.g., increasing or decreasing) a biological activity in a target cell, the method comprising:
(i) introducing into a target cell the dsDNA molecule of any of embodiments 1-170 or 314, wherein the dsDNA molecule encodes a heterologous payload that modulates a biological activity in the target cell; and
(ii) maintaining (e.g., incubating) the cell under conditions suitable for expressing the heterologous payload from the dsDNA molecule;
thereby modulating the biological activity in the target cell.

318. A method of expressing a heterologous payload in a target cell, the method comprising:
(i) providing a target cell the dsDNA molecule of any of embodiments 1-170 or 314, wherein the dsDNA molecule encodes a heterologous payload; and
(ii) maintaining (e.g., incubating) the cell under conditions suitable for expressing the heterologous payload from the dsDNA molecule;
thereby expressing the heterologous payload in the target cell.

319. A method of expressing a therapeutic payload in a target cell, the method comprising:
(i) introducing into a target cell the dsDNA molecule of any of embodiments 1-170 or 314, wherein the dsDNA molecule comprises a therapeutic payload sequence; and
(ii) maintaining (e.g., incubating) the cell under conditions suitable for expressing a therapeutic payload from the therapeutic payload sequence of the dsDNA molecule; thereby expressing the therapeutic payload in the target cell.

320. A method of delivering a heterologous payload to a target cell, the method comprising:
introducing into a target cell the dsDNA molecule of any of embodiments 1-170 or 314, wherein the double-stranded region of the dsDNA molecule comprises a sequence encoding a heterologous payload;
thereby delivering the heterologous payload to the target cell.

321. A method of delivering a therapeutic payload to a target cell, the method comprising:
introducing into a target cell the dsDNA molecule of any of embodiments 1-170 or 314, wherein the dsDNA molecule comprises a therapeutic payload sequence that encodes a therapeutic payload;
thereby delivering the therapeutic payload to the target cell.

322. A method of modulating (e.g., increasing or decreasing) a biological activity in a target cell, the method comprising:
(i) providing a target cell the dsDNA molecule of any of embodiments 1-170 or 314, wherein the dsDNA molecule encodes a heterologous payload that modulates a biological activity in the target cell; and
(ii) maintaining (e.g., incubating) the cell under conditions suitable for expressing the heterologous payload from the dsDNA molecule;
thereby modulating the biological activity in the target cell.

323. The method of embodiment 322, wherein the heterologous payload increases the biological activity in the target cell.

324. The method of embodiment 322, wherein the heterologous payload decreases the biological activity in the target cell.

325. A method of modulating (e.g., increasing or decreasing) a biological activity in a target cell, the method comprising:
(i) providing a target cell the dsDNA molecule of any of embodiments 1-170 or 314, wherein the dsDNA molecule comprises a therapeutic payload sequence encoding a therapeutic payload that modulates a biological activity in the target cell; and
(ii) maintaining (e.g., incubating) the cell under conditions suitable for expressing the therapeutic payload from the dsDNA molecule;
thereby modulating the biological activity in the target cell.

326. The method of embodiment 325, wherein the therapeutic payload increases the biological activity in the target cell.

327. The method of embodiment 325, wherein the therapeutic payload decreases the biological activity in the target cell.

328. The method of any of embodiments 317 or 322-327, wherein the biological activity comprises cell growth, cell metabolism, cell signaling, cell movement, specialization, interactions, division, transport, homeostasis, osmosis, or diffusion.

329. The method of any of embodiments 316-328, wherein the cell is an animal cell, e.g., a mammalian cell, e.g., a human cell.

330. The method of any of embodiments 316-329, which is performed ex vivo or in vivo.

331. A method of treating a cell, tissue or subject in need thereof, the method comprising:
administering to the cell, tissue or subject the dsDNA molecule of any of embodiments 1-170 or 314, or the pharmaceutical composition of any of embodiments 171-295, wherein the double-stranded region of the dsDNA molecule encodes a heterologous payload;
thereby treating the cell, tissue or subject.

332. A method of treating a cell, tissue or subject in need thereof, the method comprising:
administering to the cell, tissue or subject the dsDNA molecule of any of embodiments 1-170 or 314, or the pharmaceutical composition of any of embodiments 171-295;
thereby treating cell, tissue or the subject.

In one aspect, the invention features a dsDNA molecule, e.g., a therapeutic double stranded construct (“TDSC”).

In an embodiment, the dsDNA molecule has at least 15 nucleotides, at least 30 nucleotides, at least 50 nucleotides, at least 75 nucleotides, 100 nucleotides, at least 200 nucleotides, at least 300 nucleotides, at least 500 nucleotides, at least 750 nucleotides, at least 1,000 nucleotides, at least 2,000 nucleotides, at least 3,000 nucleotides, at least 4,000 nucleotides, at least 5,000 nucleotides, at least 6,000 nucleotides, at least 7,000 nucleotides, at least 8,000 nucleotides, at least 9,000 nucleotides, at least 10,000 nucleotides, at least 11,000 nucleotides, at least 12,000 nucleotides, at least 15,000 nucleotides, at least 20,000 nucleotides, at least 25,000 nucleotides, at least 30,000 nucleotides, at least 35,000 nucleotides, at least 40,000 nucleotides at least 45,000 nucleotides, at least 50,000 nucleotides, at least 60,000 nucleotides, or more.

In an embodiment, the dsDNA molecule has between 20 and 1000 nucleotides, between 20 and 50 nucleotides, between 100 and 500 nucleotides, between 500 and 50,000 nucleotides, between 1,000 and 50,000 nucleotides, between 2,000 and 40,000 nucleotides, between 5,000 and 50,000 nucleotides, between 500 and 50,000 nucleotides, between 500 and 25,000 nucleotides, between 1,000 and 20,000 nucleotides, between 1,000 and 10,000 nucleotides, between 10,000 and 60,000 nucleotides, between 1,000 and 20,000 nucleotides, between 1,000 and 40,000 nucleotides, between 500 and 1000 nucleotides, between 1000 and 2,000 nucleotides, between 2,000 and 3,000 nucleotides, between 3,000 and 4,000 nucleotides, between 4,000 and 5,000 nucleotides, between 5,000 and 6,000 nucleotides, between 6,000 and 7,000 nucleotides, between 7,000 and 8,000 nucleotides, between 8,000 and 9,000 nucleotides, between 9,000 and 10,000 nucleotides, between 10,000 and 11,000 nucleotides, or between 11,000 and 12,000 nucleotides.

In an embodiment, the dsDNA molecule comprises at least one nucleotide modification, e.g., a covalent nucleotide modification, e.g., selected from: 5-formylcytosine (5-formyl-2′-deoxycytosine, 5fC, f5C); 5-hydroxy-2′-deoxycytosine (5-hydroxycytosine, 5hC, h5C); 5-carboxyl-2′-deoxycytosine (5-carboxylcytosine, 5-carboxycytosine, ca5C, 5caC); 5-propargylamino-2′-deoxycytosine (5-propargylaminocytosine); 5-hydroxymethyl-2′-deoxycytosine (5-hydroxymethylcytosine, 5hmC, hm5C); glucosyl-5-hydroxymethyl-2′-deoxycytosine (glucosyl-5-hydroxymethylcytosine); 5-methyl-2′-deoxycytosine (5-methylcytosine, 5mC, m5C); phosphorothioate; or S and R phosophorothioate linkages. In some embodiments, the nucleotide modification is a base modification. In some embodiments, the nucleotide modification is a backbone modification. In some embodiments, the nucleotide modification is a sugar modification. In some embodiments, the nucleotide modification comprises a peptide conjugate. In some embodiments, the nucleotide modification comprises a protein conjugate.

In an embodiment, the effector sequence is a DNA sequence encoding a therapeutic RNA (e.g., mRNA or regulatory RNA), operably linked to a promoter. In an embodiment, the RNA can be, e.g., an mRNA, a tRNA, lncRNA, miRNA, rRNA, snRNA, microRNA, siRNA, piRNA, snoRNA, snRNA, exRNA, scaRNA, Y RNA, or hnRNA.

In an embodiment, the effector sequence is a DNA sequence encoding a therapeutic peptide or polypeptide, operably linked to a promoter. The therapeutic peptide or polypeptide may be, e.g., a DNA binding protein; an RNA binding protein; a transporter; a transcription factor; a translation factor; a ribosomal protein; a chromatin remodeling factor; an epigenetic modifying factor; an antigen; a hormone; an enzyme (such as a nuclease, e.g., an endonuclease, e.g., a nuclease element of a CRISPR system, e.g., a Cas9, dCas9, a Cas9-nickase, Cpf/Cas12a); a Crispr-linked enzyme, e.g., a base editor or prime editor; a mobile genetic element protein (e.g., a transposase, a retrotransposase, a recombinase, an integrase); a Gene Writer polypeptide; a polymerase; a methylase; a demethylase; an acetylase; a deacetylase; a kinase; a phosphatase; a ligase; a deubiquitinase; a protease; an integrase; a recombinase; a topoisomerase; a gyrase; a helicase; a lysosomal acid hydrolase); an antibody (e.g., an intact antibody, a fragment thereof, or a nanobody); a signaling peptide; a receptor ligand; a receptor; a clotting factor; a coagulation factor; a structural protein; a caspase; a membrane protein; a mitochondrial protein; a nuclear protein; or an engineered binder such as a centyrin, darpin, or adnectin. In an embodiment, the effector sequence is a DNA sequence encoding a reporter protein.

In embodiments, the dsDNA molecule can include a plurality of effector sequences. The plurality may be the same or different types, e.g., a dsDNA molecule can include an effector sequence that is a structural DNA and a second effector sequence that is a DNA sequence encoding a functional RNA or polypeptide. A dsDNA molecule can include an effector sequence that is a DNA sequence encoding a functional RNA and a second effector sequence that is a DNA sequence encoding a functional polypeptide. The plurality of effector sequences may be the same or different sequences of the same type.

In embodiments, the dsDNA molecule is not disposed in a carrier, e.g., it is formulated for naked administration.

In embodiments, the dsDNA molecule is formulated with a carrier, e.g., a lipid-based carrier, e.g., an LNP.

In embodiments, the dsDNA molecule is formulated with a pharmaceutical excipient.

In embodiments, the dsDNA molecule is formulated for parenteral administration.

In embodiments, the pharmaceutical composition is formulated for topical administration.

In embodiments, the pharmaceutical composition is substantially free of impurities or process byproducts, e.g., selected from the group consisting of: endotoxin, mononucleotides, chemically modified mononucleotides, DNA fragments or truncations, and proteins (e.g., enzymes, e.g., ligases, restriction enzymes). In some embodiments, the pharmaceutical composition is substantially free of circular DNA.

In another aspect, the invention includes a method of delivering an effector to a subject, e.g., a subject in need thereof. The method incudes administering to the subject a composition described herein, e.g., described in any embodiment above. In an embodiment, the subject has or has been diagnosed with a condition that can be treated with the effector.

In another aspect, the invention includes a method of modulating (e.g., increasing or decreasing) a biological parameter in a cell, tissue or subject. The method incudes administering to the subject a composition described herein, e.g., described in any embodiment above. In embodiments, the biological parameter is an increase or decrease in gene expression of a subject gene in a target cell, tissue or subject, which increase or decrease is effected by an effector sequence described herein. In an embodiment, the subject has or has been diagnosed with a condition that can be treated with the effector.

In another aspect, the invention includes a method of treating a cell, tissue or subject. The method includes administering to a cell, tissue or subject in need thereof a dsDNA molecule or construct described herein, e.g., described in any embodiment above. In an embodiment, the subject has or has been diagnosed with a condition that can be treated with the effector.

The disclosure also provides method of making the dsDNA molecules described herein. In an embodiment, the method comprises performing golden gate assembly.

In an embodiment, the method further comprises enriching or purifying the dsDNA molecule.

In an embodiment, the enriching or purifying includes substantially removing from the dsDNA molecule one or more impurity selected from: endotoxin, mononucleotides, chemically modified mononucleotides, single stranded DNA, DNA fragments or truncations, and proteins (e.g., enzymes, e.g., ligases, restriction enzymes).

In an embodiment, the method further comprises formulating the enriched or purified dsDNA molecule for pharmaceutical use, e.g., formulating the dsDNA molecule with a pharmaceutically acceptable excipient and/or with a carrier, e.g., an LNP.

### Definitions

As used herein, the term “alkyl” refers to a branched or unbranched saturated hydrocarbon chain having from 1 to 20 carbon atoms, or from 1 to 15 carbon atoms, or from 1 to 10 carbon atoms, or from 1 to 7 carbon atoms, or from 1 to 5 carbon atoms, or from 1 to 3 carbon atoms. In some embodiments, the alkyl group is a methyl group. The alkyl chain may be cyclic, in which case it would be known as “cycloalkyl” group.

As used herein, the term “amplicon region” refers to a particular contiguous region of a DNA molecule or an RNA molecule. In some embodiments, the amplicon region may be used as a template for PCR, using a first PCR primer and a second PCR primer that flank the amplicon region. For clarity, in such cases, the regions of the PCR template to which the first and second PCR primers bind are not included in the amplicon region. The length of an amplicon is typically given in base pairs, but an amplicon region can be found in a single stranded or double stranded nucleic acid molecule. Therefore, an amplicon region that is 200 base pairs in length can be used to refer to a double stranded region of 200 base pairs or a single stranded region of 200 nucleotides.

As used herein, the term “antibody” refers to a molecule that specifically binds to, or is immunologically reactive with, a particular antigen and includes at least the variable domain of a heavy chain, and normally includes at least the variable domains of a heavy chain and of a light chain of an immunoglobulin. Antibodies and antigen-binding fragments, variants, or derivatives thereof include, but are not limited to, polyclonal, monoclonal, multispecific, human, humanized, primatized, or chimeric antibodies, heteroconjugate antibodies (e.g., bi- tri- and quad-specific antibodies, diabodies, triabodies, and tetrabodies), single-domain antibodies (sdAb), epitope-binding fragments, e.g., Fab, Fab′ and F(ab′).sub.2, Fd, Fvs, single-chain Fvs (scFv), rIgG, single-chain antibodies, disulfide-linked Fvs (sdFv), nanobody, fragments including either a VL or VH domain, fragments produced by an Fab expression library, and anti-idiotypic (anti-Id) antibodies. Antibodies described herein can be of any type (e.g., IgG, IgE, IgM, IgD, IgA, and IgY), class (e.g., IgG1, IgG2, IgG3, IgG4, IgA1 and IgA2) or subclass of immunoglobulin molecule. Moreover, unless otherwise indicated, the term “monoclonal antibody” (mAb) is meant to include both intact molecules as well as antibody fragments (such as, for example, Fab and F(ab′)2 fragments) that are capable of specifically binding to a target protein. Fab and F(ab′)2 fragments lack the Fc fragment of an intact antibody.

As used herein, the term “carrier” means a compound, composition, reagent, or molecule that facilitates or promotes the transport or delivery of a composition (e.g., a TDSC or dsDNA molecule described herein) into a cell. For example, a carrier may be a partially or completely encapsulating agent.

As used herein, the term “chemically modified nucleotide,” as used herein with respect to DNAs, refers to a nucleotide comprising one or more structural differences relative to the canonical deoxyribonucleotides (i.e., G, T, C, and A). A chemically modified nucleotide may have (relative to a canonical nucleotide) a chemically modified nucleobase, a chemically modified sugar, a chemically modified phosphodiester linkage, or a combination thereof. No particular process of making is implied; for instance, a chemically modified nucleotide can be produced directly by chemical synthesis, or by covalently modifying a canonical nucleotide.

As used herein, the term “chemically modified cytosine nucleotide,” as used herein with respect to DNAs, refers to a chemically modified nucleotide wherein the nucleobase comprises a monocyclic 6-member ring in which carbon 4 is covalently bound to a nitrogen that is not one of the six members of the ring, wherein the nucleobase of the chemically modified cytosine nucleotide comprises one or more structural differences relative to canonical cytosine nucleobase. In some embodiments, the C-5 position of the nucleobase can have a substitution other than H. For example, the C-5 position of the nucleobase can have a substitution of —OH; -aldehyde; -carboxylic acid; -alkyl; —(CH₂)ₘOR₃, m=1-3 and R₃=H or a sugar molecule; or -propargylamino. In some embodiments, the chemically modified cytosine nucleotide further comprises a chemical modification on the sugar or phosphodiester linkage. No particular process of making is implied.

As used herein, the term “closed end” refers to a portion of a DNA molecule positioned at one end of a double-stranded region, in which all nucleotides within the portion of the DNA molecule are covalently attached to adjacent nucleotides on either side. A closed end may, in some embodiments, include a loop comprising one or more nucleotides that are not hybridized to another nucleotide. In some embodiments, every nucleotide of the closed end is hybridized to another nucleotide. In some embodiments, a dsDNA molecule (e.g., TDSC) comprises a first closed end (e.g., upstream of a heterologous object sequence) and a second closed end (e.g., downstream of a heterologous object sequence).

As used herein, the term “open end” refers to a portion of a DNA molecule positioned at one end of a double-stranded region, in which at least one nucleotide (a “terminal nucleotide”) is covalently attached to exactly one other nucleotide. In some embodiments, the terminal nucleotide comprises a free 5′ phosphate. In some embodiments, the terminal nucleotide comprises a free 3′ OH. In some embodiments, in a dsDNA molecule comprising a first DNA strand and a second DNA strand, the open end comprises a first terminal nucleotide on the first DNA strand and a second terminal nucleotide on the second DNA strand. In some embodiments, a dsDNA molecule comprises a first open end (e.g., upstream of a heterologous object sequence) and a second open end (e.g., downstream of a heterologous object sequence). In some embodiments, the open end comprises a blunt end, a sticky end, or a Y-adaptor.

As used herein, the term “desired DNA sequence” refers to the DNA sequence that a user intends to produce. In some embodiments, the desired DNA sequence is the sequence of an amplicon region in a PCR template. As is clear from context, in some embodiments (e.g., when all the dsDNA molecules in a sub-population have the same DNA sequence which is a desired DNA sequence) a DNA molecule has a desired DNA sequence throughout its whole length. In other embodiments, a DNA molecule may have a desired DNA sequence in a specified region (e.g., an amplicon region) of the DNA molecule and have one or more errors outside that region.

As used herein, the term “desired RNA sequence” refers to the RNA sequence that a user intends to produce. In some embodiments, the desired RNA sequence is the sequence produced by error-free transcription of a desired DNA sequence. As is clear from context, in some embodiments an RNA molecule has a desired RNA sequence throughout its whole length, while in other embodiments, an RNA molecule may have a desired RNA sequence in a specified region (e.g., an amplicon region) of the RNA molecule and have one or more errors outside that region.

As used herein, the term “DNA” refers to any compound and/or substance that comprises at least two (e.g., at least 10, at least 20, at least 50, at least 100) covalently linked deoxyribonucleotides. In some embodiments, the DNA is a single oligonucleotide chain, while in other embodiments, the DNA comprises a plurality of oligonucleotide chains, while in yet other embodiments the DNA is a portion of an oligonucleotide chain. In some embodiments, DNA is a compound and/or substance that is or can be incorporated into an oligonucleotide chain via a phosphodiester linkage. In some embodiments, the DNA comprises solely canonical nucleotides. In some embodiments, the DNA comprises one or more chemically modified nucleotides. In some embodiments, at least 80%, at least 85%, at least 90%, at least 95%, at least 98%, at least 99%, or 100% of the sugars of the DNA are deoxyribose sugars. In some embodiments, the DNA was prepared by one or more of: isolation from a natural source, enzymatic synthesis by polymerization based on a complementary template (in vivo or in vitro), reproduction in a recombinant cell or system, and chemical synthesis.

As used herein, the term “DNA endform” refers to a structure comprising DNA that is situated at an end of a dsDNA molecule (e.g., a TDSC). In some embodiments, the DNA end form comprises a closed end. In other embodiments, the DNA end form comprises an open end. In some embodiments, the DNA end form comprises a hairpin, a loop, a Y-adaptor, a blunt end, or a sticky end. The DNA end form may comprise one or both of a single stranded region and a double stranded region. The DNA end form may comprise canonical nucleotides, chemically modified nucleotides, or a combination thereof. In some embodiments, the DNA end form comprises between 3-100 nucleotides. In some embodiments, the dsDNA molecule comprises a first DNA end form at a first end and a second DNA end form at a second end. In some embodiments, the first DNA end form and the second DNA end form of a dsDNA molecule are the same type. In some embodiments, the first DNA end form and the second DNA end form of a dsDNA molecule are different types.

As used herein, with respect to a nucleic acid sequence, the term “error” refers to a nucleotide sequence difference in a nucleic acid sequence relative to a desired RNA or DNA sequence. In some embodiments, the error is a substitution, an insertion, or a deletion. In some embodiments, the error may be introduced by PCR. In some embodiments, the error may be introduced by a polymerase, e.g., a DNA polymerase or an RNA polymerase. For avoidance of doubt, a replacement of a canonical cytosine with a chemically modified cytosine nucleotide is not considered an error.

As used herein, the term “exonuclease-resistant”, when used to describe a DNA, means that the DNA, if it comprises closed ends, is resistant to the exonuclease assay described in Example 2, and if it comprises an open end (e.g., two open ends), is resistant to the exonuclease assay described in Example 3.

As used herein, the term “heterologous”, when used to describe a first element in reference to a second element means that the first element and second element do not exist in nature disposed as described. For example, a heterologous polypeptide, nucleic acid molecule, construct or sequence refers to (a) a polypeptide, nucleic acid molecule or portion of a polypeptide or nucleic acid molecule sequence that is not native to a cell in which it is expressed, (b) a polypeptide or nucleic acid molecule or portion of a polypeptide or nucleic acid molecule that has been altered or mutated relative to its native state, or (c) a polypeptide or nucleic acid molecule with an altered expression as compared to the native expression levels under similar conditions. For example, a heterologous regulatory sequence (e.g., promoter, enhancer) may be used to regulate expression of a gene or a nucleic acid molecule in a way that is different than the gene or a nucleic acid molecule is normally expressed in nature. In another example, a heterologous domain of a polypeptide or nucleic acid sequence (e.g., a DNA binding domain of a polypeptide or nucleic acid encoding a DNA binding domain of a polypeptide) may be disposed relative to other domains or may be a different sequence or from a different source, relative to other domains or portions of a polypeptide or its encoding nucleic acid. In certain embodiments, a heterologous nucleic acid molecule may exist in a native host cell genome, but may have an altered expression level or have a different sequence or both. In other embodiments, heterologous nucleic acid molecules may not be endogenous to a host cell or host genome but instead may have been introduced into a host cell by transformation (e.g., transfection, electroporation), wherein the added molecule may integrate into the host genome or can exist as extra-chromosomal genetic material either transiently (e.g., mRNA) or semi-stably for more than one generation (e.g., episomal viral vector, plasmid or other self-replicating vector).

As used herein, the term “heterologous functional sequence” refers to a nucleic acid sequence that is heterologous to an adjacent (e.g., directly adjacent) nucleic acid sequence and has one or more biological function. In some embodiments, the biological function comprises targeting to an organelle, e.g., nuclear targeting. In some embodiments, the heterologous functional sequence comprises a nuclear targeting sequence or a regulatory sequence.

As used herein, the terms “increasing” and “decreasing” refer to modulating resulting in, respectively, greater or lesser amounts, of function, expression, or activity of a metric relative to a reference. For example, subsequent to administration of a dsDNA molecule in a method described herein, the amount of the metric described herein (e.g., the level of gene expression, or a marker of innate immunity) may be increased or decreased in a subject by at least 5%, 10%, 15%, 20%, 25%, 30%, 35%, 40%, 45%, 50%, 55%, 60%, 65%, 70%, 75%, 80%, 85%, 90%, 95% or 98% or more relative to the amount of the marker prior to administration, or relative to administration of a control dsDNA molecule, such as a dsDNA molecule comprising chemically modified nucleotides compared to a control dsDNA molecule having only unmodified nucleotides. Generally, the metric is measured subsequent to administration at a time that the administration has had the recited effect, e.g., at least one day, one week, one month, 3 months, or 6 months, after a treatment regimen has begun.

As used herein the term “linear” in reference to a dsDNA molecule (e.g., TDSC) described herein, means a nucleic acid comprising two DNA strands or portions of strands which hybridize with each other (thereby forming a double stranded region), wherein the structure comprises two ends. An end may be a closed end or an open end. The two strands that hybridize with each other may be partially or completely complementary. In some embodiments, a linear dsDNA molecule consists of a single strand of DNA that is circular under denaturing conditions, wherein under physiological conditions a first portion of the strand hybridizes to a second portion of the strand (thereby forming a double stranded region), and the linear dsDNA molecule comprises a first closed end comprising a first loop and a second closed end comprising a second loop.

As used herein, the term “loop” refers to a nucleic acid sequence that is single stranded. A loop is connected at both ends by a double stranded region referred to as a “stem”, to form a “stem-loop”.

As used herein, the term “maintenance sequence” is a DNA sequence or motif that enables or facilitates retention of a DNA molecule in the nucleus through cell division. A maintenance sequence typically enables replication and/or transcription of DNA in the nucleus by interacting with proteins that facilitate chromatin looping. An example of a maintenance sequence is a scaffold/matrix attached region (S/MAR element).

As used herein, a “nuclear targeting sequence” is a DNA sequence that enables or facilitates DNA entry into a target cell nucleus.

As used herein, a “pharmaceutical composition” or “pharmaceutical preparation” is a composition or preparation which is indicated for animal, e.g., human or veterinary pharmaceutical use, for example, non-human animal or human prophylactic or therapeutic use. A pharmaceutical preparation comprises an active agent having a biological effect on a cell or tissue of a subject, e.g., having pharmacological activity or an effect in the mitigation, treatment, or prevention of disease, in combination with a pharmaceutically acceptable excipient or diluent. A pharmaceutical composition also means a finished dosage form or formulation of a prophylactic or therapeutic composition.

As used herein, the terms “peptide,” “polypeptide,” and “protein” are used interchangeably and refer to a compound comprising amino acid residues covalently linked by peptide bonds, or by means other than peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds or by means other than peptide bonds. As used herein, the term refers to both short chains, which also commonly are referred to in the art as peptides, oligopeptides and oligomers, for example, and to longer chains, which generally are referred to in the art as proteins, of which there are many types. In some embodiments, a polypeptide comprises a non-canonical amino acid residue.

As used herein, the term “propargylamino” refers to the functional group of —C≡CCH₂NH₂.

As used herein, the term “protelomerase sequence” refers to a nucleotide sequence capable of being generated by a protelomerase that joins a first protelomerase recognition sequence (PRS) to a second PRS. In some embodiments, the protelomerase sequence was produced by a process involving protelomerase, and in other embodiments the protelomerase sequence was produced by a process that does not involve protelomerase (e.g., by solid phase synthesis).

As used herein, a “sense strand” of a dsDNA is a strand which has the same sequence as an mRNA or pre-mRNA which encodes for a functional protein, and does not serve as a template for transcription. An “antisense strand” of a dsDNA is a strand that has a sequence complementary to an mRNA or pre-mRNA which encodes for a functional protein and/or can serve as a template for transcription.

As used herein, the term “double stranded DNA molecule” or dsDNA molecule means a DNA composition comprising two complementary chains of deoxyribonucleotides that base pair to each other. The two complementary strands may have perfect complementarity or may have one or more mismatches, e.g., forming bulges. Either of the two strands may, in some embodiments, have paired regions of self-complementarity that form intramolecular/intrastrand double stranded motifs in a folded configuration, for example, may form hairpin loops, junctions, bulges or internal loops. In some embodiments, the dsDNA molecule is circular or linear. In some embodiments, the dsDNA molecule comprises one or two closed ends. In some embodiments (e.g., in a dsDNA molecule with closed ends) the two complementary chains of deoxyribonucleotides are covalently linked. In some embodiments, the dsDNA molecule is a TDSC.

As used herein, the term “therapeutic double stranded construct” (“TDSC”) refers to a linear construct comprising DNA, wherein the construct is at least partially double stranded. A TDSC does not comprise a plasmid backbone sequence (e.g., does not comprise a bacterial origin of replication). A TDSC does not comprise a viral capsid or a viral envelope. In some embodiments, the TDSC comprises a closed end or an open end (e.g., a blunt end or a sticky end). In some embodiments, the TDSC is suitable for administration to a human subject.

As used herein, the term “terminal nucleotide” refers to a nucleotide that is covalently attached to exactly one other nucleotide. In some embodiments, the terminal nucleotide comprises a free 5′ phosphate. In some embodiments, the terminal nucleotide comprises a free 3′ OH.

As used herein, “treatment” and “treating” refer to the medical management of a subject with the intent to improve, ameliorate, stabilize (i.e., not worsen), prevent or cure a disease, pathological condition, or disorder. This term includes active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to minimizing or partially or completely inhibiting the development of the associated disease, pathological condition, or disorder); and supportive treatment (treatment employed to supplement another therapy). Treatment also includes diminishment of the extent of the disease or condition; preventing spread of the disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

As used herein, the term “Y-adaptor” refers to a nucleic acid structure comprising a first nucleic acid region and a second nucleic acid region which are complementary (e.g., perfectly complementary) to each other; the first and second regions may hybridize to form a double stranded region. The first nucleic acid region is covalently linked to a third nucleic acid region, and the second nucleic acid region is covalently linked to a fourth nucleic acid region, and the third and fourth nucleic acid regions are not substantially complementary to each other; the third and fourth regions may be single stranded. The first nucleic acid region is 3′ of the third nucleic acid region and the second nucleic acid region is 5′ of the fourth nucleic acid region. As a result, the third and fourth regions may be situated on the same side of the double stranded regions. The Y-adaptor may be part of a dsDNA molecule.

### BRIEF DESCRIPTION OF THE DRAWINGS

FIGS. 1A-1B are a series of diagrams showing exemplary covalently-closed DNA end forms that can be included in a dsDNA molecule, e.g., therapeutic double-stranded construct (TDSC), as described herein (e.g., at one or both ends of the dsDNA molecule). Shown in (A) are exemplary dsDNA molecules, e.g., TDSCs, comprising no loop ends (e.g., protelomerase sequences), inverted terminal repeats (ITRs), or hairpins at the ends, which can be made up of unmodified nucleotides (white symbols) or may comprise chemically modified nucleotides (gray symbols). Chemically modified nucleotides can include nucleotides modified, for example, in the backbone, sugar, or base, or nucleotides that are conjugated to a peptide or protein. In some instances, both of the DNA strands are unmodified. In some instances, both of the DNA strands are chemically modified. In some instances, the antisense strand is chemically modified. In some instances, the sense strand is chemically modified. The solid-line box indicates a dsDNA molecule that is covalently closed with hairpins at the ends, e.g., a linear, covalently closed dsDNA molecule with end forms comprising phosphorothioate modifications. The dashed-line box indicates a dsDNA molecule that is covalently closed with no loop ends, e.g., a linear, covalently closed dsDNA molecule with TelN end forms.

FIG. 2 is a series of diagrams showing double-stranded DNA constructs, including exemplary dsDNA molecules, e.g., TDSCs, comprising exemplary DNA end forms (e.g., at one or both ends) that are not covalently closed. Such exemplary dsDNA molecules, e.g., TDSCs, can comprise a Y end (e.g., a Y adaptor, e.g., as described herein). The DNA end forms can, in some instances, be made up of unmodified nucleotides (white symbols). In some instances, the DNA end forms comprise chemically modified nucleotides (gray symbols). Chemically modified nucleotides can include nucleotides modified, for example, in the backbone, sugar, or base, or nucleotides that are conjugated to a peptide or protein. In some instances, both of the DNA strands are unmodified. In some instances, both of the DNA strands are chemically modified. In some instances, the antisense strand is chemically modified. In some instances, the sense strand is chemically modified. Also shown in the upper right is an exemplary DNA construct lacking DNA end forms or chemical modifications (i.e., an unmodified double-stranded DNA molecule).

FIGS. 3A and 3B depict gel electrophoresis images of PCR products from reactions using unmodified dCTP (denoted as “0%”), or when 5-formyl-dCTP was added to the PCR mix at a 1:3 ratio to unmodified dCTP (denoted as “25%”). PCR was performed using either Deep Vent polymerase (FIG. 3A) or KOD Xtreme polymerase (FIG. 3B). Arrow indicates the desired product.

FIG. 4 is a diagram depicting production of covalently closed linear dsDNA molecules with end forms comprising phosphorothioate modifications. FIG. 4 discloses SEQ ID NOS 86-87, respectively, in order of appearance.

FIG. 5 is a diagram depicting production of covalently closed linear dsDNA molecules with TelN end forms. FIG. 5 discloses SEQ ID NOS 88-89, 58 and 57, respectively, in order of appearance.

FIG. 6 is a diagram depicting circular dsDNA molecules with or without chemical modifications.

FIG. 7 is a diagram depicting an exemplary method of production of circular dsDNA molecules. A linear dsDNA molecule may be contacted with a restriction enzyme (e.g., KpnI) that creates compatible sticky ends which may then be joined to each other by ligation, producing a circular dsDNA. FIG. 7 discloses SEQ ID NOS 90, 92, 91 and 93, respectively, in order of appearance.

FIG. 8 is a gel electrophoresis image of chemically modified DNA molecules, including modifications at the C-5 position of cytosine. Lane 1 shows unmodified control dsDNA molecules. Lane 2 shows dsDNA molecules produced in a reaction using 25% 5-formyl-dCTP. Lane 3 shows dsDNA molecules produced in a reaction using 50% 5-hydroxy-dCTP.

FIG. 9 shows fragment analyzer traces of circular dsDNA produced in a reaction using 25% 5-formyl-dCTP.

FIG. 10 shows fragment analyzer traces of linear covalently closed dsDNA molecules with end forms comprising phosphorothioate modifications produced in a reaction using 25% 5-formyl-dCTP.

FIG. 11 shows fragment analyzer traces of linear covalently closed dsDNA molecules with TelN end forms produced in a reaction using 25% 5-formyl-dCTP.

FIG. 12 shows fragment analyzer traces of linear covalently closed dsDNA molecules with end forms comprising phosphorothioate modifications produced in a reaction using 50% 5-hydroxy-dCTP.

FIG. 13 is a graph showing the proportion of HepG2, HEKa, or U937 cells expressing mCherry following lipofection with a circular dsDNA construct comprising 5-formylcytosine modifications produced in a reaction using 25% 5-formyl-dCTP, relative to cells lipofected with an unmodified dsDNA construct.

FIGS. 14A-14C are a series of graphs showing the relative mRNA levels of IFNβ, CXCL10, and IL6 in HEKa cells following lipofection with circular dsDNA molecules comprising 5-formylcytosine modifications produced in a reaction using 25% 5-formyl-dCTP, or unmodified dsDNA molecules. The mRNA level fold change versus method control (i.e., transfection with no DNA) across all time points (6 h, 24 h, and 72 h post-transfection) are shown.

FIGS. 15A-15C are a series of graphs showing the mRNA levels of IFNβ, CXCL10, and IL6 in U937 cells following transfection with circular dsDNA molecules comprising 5-formylcytosine modifications produced in a reaction using 25% 5-formyl-dCTP, or unmodified dsDNA molecules. Dashed horizontal line denotes method control (i.e., transfection with no DNA). The mRNA level fold change versus method control across all time points (6 h, 24 h, and 72 h post-transfection) are shown.

FIG. 16 is a scatterplot showing the innate immune response of HEKa cells to linear dsDNA molecules comprising phosphorothioated modifications and comprising 5-hydroxycytosine (produced in a reaction using 50% 5-hydroxy-dCTP), 5-formylcytosine (produced in a reaction using 25% 5-formyl-dCTP), or other modifications. X-axis represents reduction in interferon signaling, as defined as the average fold-change reduction of markers IFNB and CXCL10 relative to unmodified DNA. Y-axis represents reduction in inflammatory cytokine signaling, as defined as the average fold-change reduction of markers IL6 and TNFa relative to unmodified DNA. The dashed-line oval indicates other dsDNA chemical modifications (n=42).

FIG. 17 is a scatterplot showing the innate immune response of HEKa cells to linear dsDNA molecules comprising phosphorothioated end adapters and the indicated modification at the C-5 position of cytosine (i.e., 5-formylcytosine, 5-hydroxycytosine, glucosyl-5-hydroxymethylcytosine, 5-carboxycytosine, 5-propargylaminocytosine, 5-methylcytosine, or 5-hydroxymethylcytosine). X-axis represents reduction in interferon signaling, defined as the average fold-change reduction of markers IFNB and CXCL10 relative to unmodified DNA. Y-axis represents reduction in inflammatory cytokine signaling, defined as the average fold-change reduction of markers IL6 and TNFa relative to unmodified DNA. The dsDNA molecules were produced in reactions designed for 25% incorporation of 5-formylcytosine, 50% incorporation of 5-hydroxycytosine, 50% incorporation of glucosyl-5-hydroxymethylcytosine, 60% incorporation of 5-carboxycytosine, 25% incorporation of 5-propargylaminocytosine, 75% incorporation of 5-methylcytosine, or 50% incorporation of 5-hydroxymethylcytosine.

FIG. 18 is a scatterplot showing the innate immune response to linear dsDNA molecules with phosphorothioated end adapters, and reporter gene expression of constructs containing 5-hydroxycytosine (produced in a reaction using 50% 5-hydroxy-dCTP), 5-formylcytosine (produced in a reaction using 25% 5-formyl-dCTP), or other chemical modifications. X-axis represents reduction in innate immune signaling, defined as the average fold-change reduction of markers IFNB, CXCL10, IL6, and TNFa relative to unmodified dsDNA molecules. Y-axis represents relative reporter gene expression, defined as the proportion of mCherry+ cells relative to unmodified control DNA. The dashed-line oval indicates other dsDNA chemical modifications (n=38).

FIG. 19 is a pair of graphs showing the function (here, ability to produce a protein, in this case mCherry) of hemi-modified dsDNA produced in a reaction using 100% 5-hydroxycytosine (5hC100), measured as total fluorescence over background (left panel) or % mCherry+ cells (right panel).

FIG. 20 is a pair of graphs showing the immune steath characteristics of hemi-modified dsDNA produced in a reaction using 100% 5-hydroxycytosine (5hC100), based on IL6 levels (left panel) or CXCL10 levels (right panel). In FIGS. 19 and 20, P6 unmodified indicates a closed end dsDNA with phosphorothioate but lacking modified nucleobases.

### DETAILED DESCRIPTION

This disclosure relates to compositions and methods for providing an effector, e.g., a therapeutic effector, to a cell, tissue or subject, e.g., in vivo or in vitro. The effector may be a DNA sequence, a polypeptide, e.g., a therapeutic protein; or an RNA, e.g., a regulatory RNA or an mRNA.

### Chemically Modified Nucleotides

A dsDNA molecule described herein may comprise a chemically modified nucleotide, such as a chemically modified cytosine nucleotide. Without wishing to be bound by theory, in some embodiments the chemically modified cytosine nucleotides described herein increase the “stealth” of a dsDNA molecule to an immune response, while supporting expression of a gene on the dsDNA molecule. Exemplary chemically modified cytosine nucleotides are provided below. A nucleobase comprising 5-formylcytosine is shown below as Formula II.

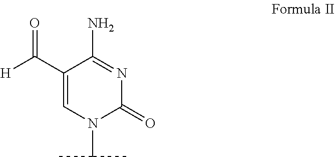

A nucleobase comprising 5-hydroxycytosine is shown below as Formula III.

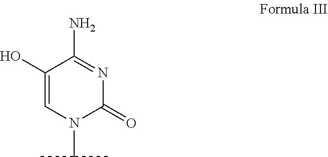

A nucleobase comprising 5-carboxycytosine is shown below as Formula IV.

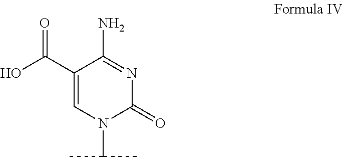

A nucleobase comprising 5-propargylaminocytosine is shown below as Formula V.

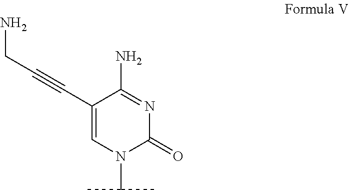

A nucleobase comprising 5-methylcytosine is shown below as Formula VI.

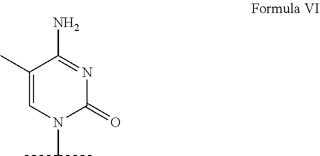

A nucleobase comprising 5-hydroxymethylcytosine is shown below as Formula VII.

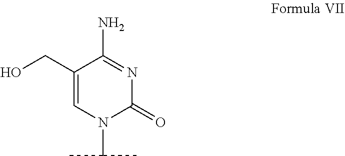

A nucleobase comprising glucosyl-5-hydroxymethylcytosine is shown below as Formula VIII.

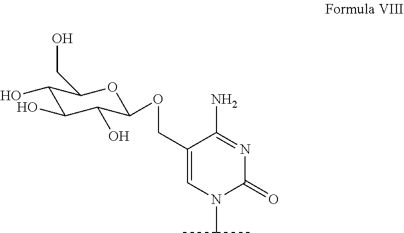

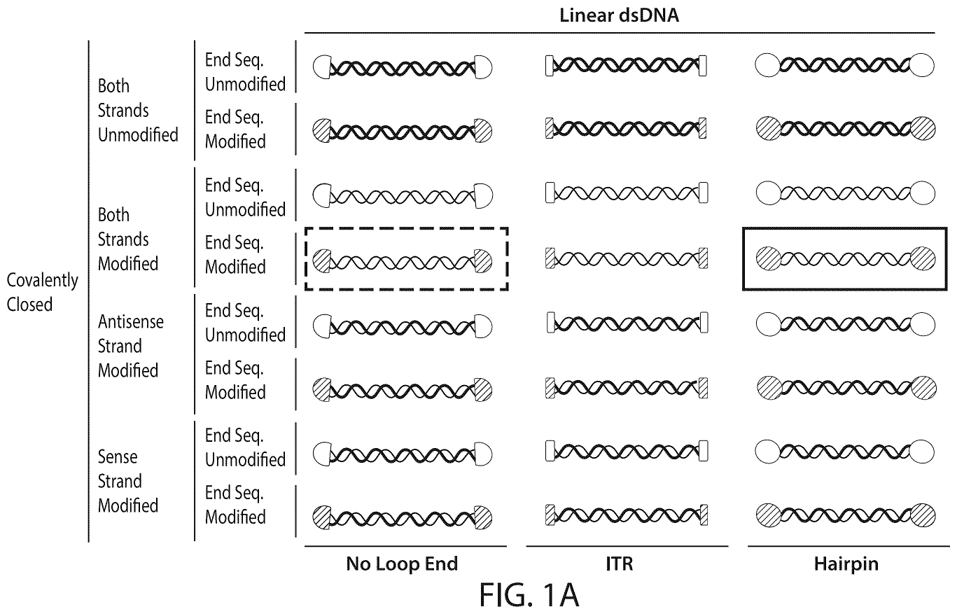

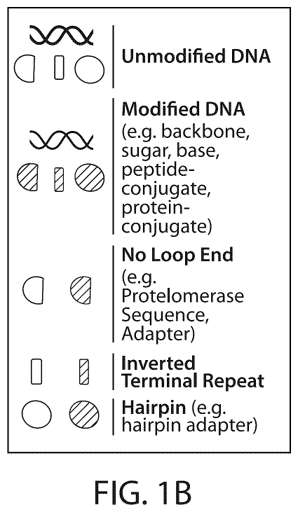

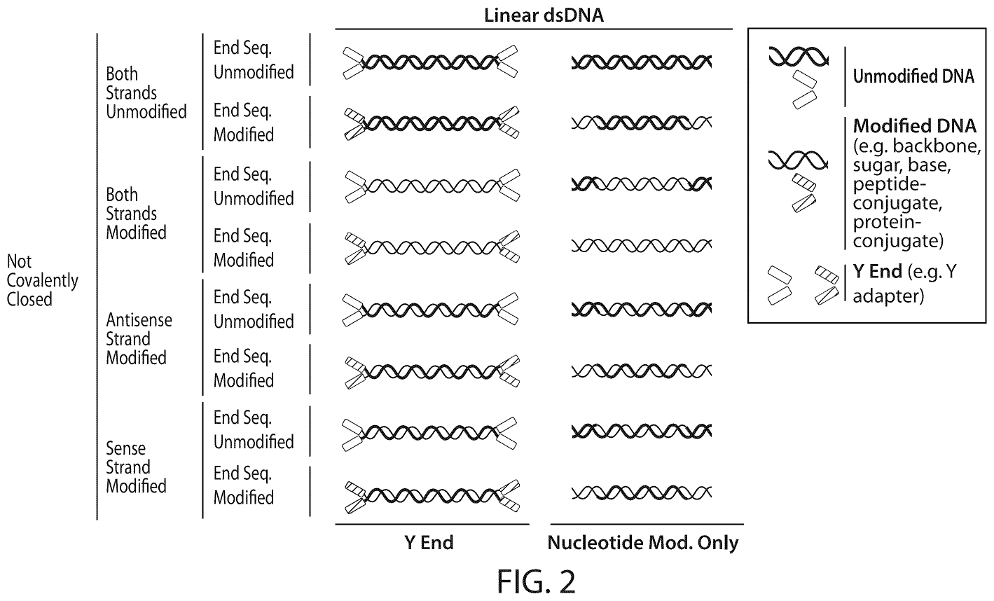

The dsDNA molecule (e.g., TDSC) compositions described herein may have chemical modifications of the nucleobases, sugars, and/or the phosphate backbone (e.g., as shown in FIGS. 1A-2). While not wishing to be bound by theory, such modifications can be useful for protecting a DNA from degradation (e.g., from exonucleases) or from the immune system of a host tissue or subject. In general, a chemically modified nucleotide has the same base-pairing specificity as the unmodified nucleotide, e.g., a chemically modified adenine “A” can base-pair with thymine “T”. In certain embodiments, chemical modifications (e.g., one or more modifications) are present in each of the sugar and the internucleoside linkage.

In some embodiments, the dsDNA molecule (e.g., TDSC) comprises at least one chemical modification. Examples of chemical modifications to DNA useful in the methods described herein include, e.g., 5-formylcytosine (5-formyl-2′-deoxycytosine, 5fC, f5C); 5-hydroxy-2′-deoxycytosine (5-hydroxycytosine, 5hC, h5C); 5-carboxyl-2′-deoxycytosine (5-carboxylcytosine, 5-carboxycytosine, ca5C, 5caC); 5-propargylamino-2′-deoxycytosine (5-propargylaminocytosine); 5-hydroxymethyl-2′-deoxycytosine (5-hydroxymethylcytosine, 5hmC, hm5C); glucosyl-5-hydroxymethyl-2′-deoxycytosine (glucosyl-5-hydroxymethylcytosine); 5-methyl-2′-deoxycytosine (5-methylcytosine, 5mC, m5C); phosphorothioate; or S and R phsophorothioate linkages. See, e.g., Pu et al. 2020. An in-vitro DNA phosphorothioate modification reaction. Mol Microbiol. 113: 452-463; Zheng & Sheng. 2021.

In some embodiments, a dsDNA molecule as described herein may comprise a phosphorothioate-modified nucleotide. In some embodiments, a DNA end form (e.g., an exonuclease-resistant DNA end form) as described herein may comprise a phosphorothioate-modified nucleotide. In some embodiments, the dsDNA molecules described herein may include S and R phosphorothioate modified nucleotide linkages. In one embodiment, the phosphorothioate linkages are made according to Iwamoto et al, 2017, Nature Biotechnology, Volume 35:845-851. Briefly, monomers of nucleoside 3′-oxazaphospholidine derivates undergo stereocontrolled oligonucleotide synthesis with iterative capping and sulfurization to create stereocontrolled phosphorothioate linkages. The final sample is analyzed by reverse-phase high-performance liquid chromatography (RP-HPLC) and Ultraperformance liquid chromatography mass spectrometry (UPLC/MS) to determine stereochemistry of the modification. Nucleic acids containing phosphorothioate linkages are also commercially available.

In some embodiments, the dsDNA molecules described herein may include 5-methylcytosine modified nucleotides, e.g., made following the methods in Lin et al, 2002, Mol Cell Biol, Volume 22, Issue 3:704-723. Briefly, cytosine or the sequence containing cytosine is incubated with glutathione S-transferase fusion of wild-type Dnmt3a (GST-3a) protein using unlabeled S-adenosylmethionine (AdoMet). The nucleotides are purified and analyzed by HPLC to determine that the nucleotides are methylated at the correct position. 5-methylcytosine modified nucleotides are also available commercially.

In some embodiments, a dsDNA molecule described herein comprises a carboxyl modification or a formyl modification.

In embodiments, a dsDNA molecule described herein, or one strand (e.g., the sense strand or the antisense strand) of the dsDNA molecule, comprises between 1-100% chemically modified nucleotides, between 1%-90% chemically modified nucleotides, between 1%-80% chemically modified nucleotides, between 1%-70% chemically modified nucleotides, between 1%-60% chemically modified nucleotides, between 1%-50% chemically modified nucleotides, between 1%-40% chemically modified nucleotides, between 1%-30% chemically modified nucleotides, between 1%-20% chemically modified nucleotides, between 1%-15% chemically modified nucleotides, between 1%-10% chemically modified nucleotides, between 20%-90% chemically modified nucleotides, between 20%-80% chemically modified nucleotides. In embodiments, a dsDNA molecule described herein, or one strand (e.g., the sense strand or the antisense strand) of the dsDNA molecule, comprises at least 1% chemically modified nucleotides, at least 5% chemically modified nucleotides; at least 10% chemically modified nucleotides; at least 15% chemically modified nucleotides; at least 20% chemically modified nucleotides; at least 25% chemically modified nucleotides; at least 30% chemically modified nucleotides; at least 40% chemically modified nucleotides; at least 50% chemically modified nucleotides; at least 60% chemically modified nucleotides; at least 70% chemically modified nucleotides; at least 80% chemically modified nucleotides; at least 85% chemically modified nucleotides; at least 90% chemically modified nucleotides; at least 92% chemically modified nucleotides; at least 95% chemically modified nucleotides; at least 97% chemically modified nucleotides. In embodiments, a dsDNA molecule described herein, or one strand (e.g., the sense strand or the antisense strand) of the dsDNA molecule, comprises chemically modified nucleotides at between 0-100%, 10%-100%, 20%-100%, 30%-100%, 40%-100%, 50%-100%, 60%-100%, 10%-50% of cytosines.

In embodiments, chemically modified nucleotides, e.g., modifications described herein, can be introduced in the dsDNA molecules described herein throughout the entire sequence; within an element of a sequence, e.g., an element described herein; at a 5′- or 3′-end; and/or between the last 10, 8, 6, 5, 4, 3, or 2 nucleotides at the 5′- or 3′-end.

In some embodiments, a dsDNA molecule as described herein comprises chemically modified nucleotides on only one strand (e.g., as shown in FIG. 1A). In some embodiments, a dsDNA molecule as described herein comprises chemically modified nucleotides on the antisense strand. In some embodiments, a dsDNA molecule as described herein comprises chemically modified nucleotides on the sense strand.

In some embodiments, a dsDNA molecule as described herein comprises chemically modified nucleotides on both strands (e.g., as shown in FIGS. 1A and 2). In certain embodiments, both strands comprise chemical modifications at the same positions (e.g., chemically modified nucleotides on one strand are base-paired with chemically modified nucleotides on the opposite strand, and/or non-chemically modified nucleotides on one strand are base-paired with non-chemically modified nucleotides on the opposite strand). In embodiments, the entirety of both strands are composed of chemically modified nucleotides. In other embodiments, the two strands of a dsDNA molecule as described herein comprise different chemical modification patterns (e.g., one or more chemically modified nucleotides on one strand are base-paired with non-chemically modified nucleotides on the other strand). In embodiments, a dsDNA molecule as described herein comprises one or more double-stranded regions in which both strands are chemically modified, and/or one or more double-stranded regions in which neither strand is chemically modified. In embodiments, a dsDNA molecule as described herein comprises one or more double-stranded regions in which one strand is chemically modified and the other is not.

In embodiments, a dsDNA molecule as described herein comprises one or more DNA end forms (e.g., exonuclease-resistant DNA end forms, e.g., covalently closed DNA end forms or non-covalently closed DNA end forms, e.g., as described herein) that each comprise one or more chemically-modified nucleotides (e.g., on one or both strands of the DNA end form). In embodiments, a dsDNA molecule comprises a double-stranded region flanked by non-covalently closed exonuclease-resistant DNA end forms comprising chemically-modified nucleotides, e.g., as described herein (e.g., in FIG. 2).

In embodiments, a dsDNA molecule described herein has one or more chemical modification that disrupts the ability of a portion of the dsDNA molecule to form a double stranded structure, e.g., a dsDNA molecule described herein has one or more chemical modification on a nucleotide present in a region having intramolecular complementarity. In embodiments, a dsDNA molecule described herein has one or more chemical modification that disrupts base pairing of regions of intramolecular complementarity relative to the unmodified sequence of the dsDNA molecule. In some embodiments the chemically modified nucleotides used herein have a reduced propensity to base-pair with chemically modified nucleotides compared to the propensity of unmodified nucleotides to base pair with unmodified nucleotides. In some embodiments the chemically modified nucleotides used herein have an increased propensity to base-pair with unmodified nucleotides compared to modified nucleotides.

In some embodiments, a chemically modified dsDNA molecule described herein exhibits decreased recognition by DNA sensors in a host tissue or subject compared to an unmodified dsDNA molecule of the same sequence, e.g., at least 10%, at least 20%, at least 30%, at least 40%, at least 50%, at least 60%, at least 70%, at least 80%, at least 90%, at least 95% or more decreased recognition by DNA sensors in a host tissue or subject compared to an unmodified dsDNA molecule of the same sequence. In some embodiments, a chemically modified dsDNA molecule described herein exhibits decreased recognition by DNA sensors in a host tissue or subject compared to an unmodified dsDNA molecule of the same sequence, e.g., 10%-20%, 20%-30%, 30%-40%, 40%-50%, 50%-60%. 60%-70%, 70%-80%, 80%-90%, or 90%-95%, decreased recognition by DNA sensors in a host tissue or subject compared to an unmodified dsDNA molecule of the same sequence. In some embodiments, a chemically modified dsDNA molecule described herein exhibits decreased degradation by DNA nucleases compared to an unmodified dsDNA molecule of the same sequence, e.g., at least 10%, at least 20%, at least 30%, at least 40%, at least 50%, at least 60%, at least 70%, at least 80%, at least 90%, at least 95% or more decreased degradation by DNA nucleases in a host tissue or subject compared to an unmodified dsDNA molecule. In some embodiments, a chemically modified dsDNA molecule described herein exhibits decreased degradation by DNA nucleases compared to an unmodified dsDNA molecule of the same sequence, e.g., 10%-20%, 20%-30%, 30%-40%, 40%-50%, 50%-60%. 60%-70%, 70%-80%, 80%-90%, or 90%-95% decreased degradation by DNA nucleases in a host tissue or subject compared to an unmodified dsDNA molecule. In some embodiments, a chemically modified dsDNA molecule described herein shows decreased activation of the innate immune system in a target/host tissue or subject compared to an unmodified dsDNA molecule of the same sequence, e.g., at least 10%, at least 20%, at least 30%, at least 40%, at least 50%, at least 60%, at least 70%, at least 80%, at least 90%, at least 95% or more decreased activation of the innate immune system in a target/host tissue or subject compared to an unmodified dsDNA molecule of the same sequence. In some embodiments, a chemically modified dsDNA molecule described herein shows decreased activation of the innate immune system in a target/host tissue or subject compared to an unmodified dsDNA molecule of the same sequence, e.g., 10%-20%, 20%-30%, 30%-40%, 40%-50%, 50%-60%. 60%-70%, 70%-80%, 80%-90%, or 90%-95% decreased activation of the innate immune system in a target/host tissue or subject compared to an unmodified dsDNA molecule of the same sequence.

In some embodiments, a dsDNA molecule comprising chemically modified nucleotides described herein exhibits any of the following properties in a target/host tissue or subject compared to dsDNA of the same sequence that does not comprise chemically modified nucleotides (unmodified dsDNA): increased integration of exogenous construct in genome of target cell; increased retention in a target cell through replication; reduced secondary or tertiary structure formation; reduced interaction with innate immune sensors; reduced interaction with nucleases; enhanced stability; enhanced longevity; reduced toxicity; enhanced delivery; increased expression; increased transport across membranes; increased binding to DNA binding moieties such as nuclear DNA binding proteins, transcription factors, chaperones, DNA polymerases. In embodiments, any of the above listed properties is modulated at least 10%, at least 20%, at least 30%, at least 40%, at least 50%, at least 60%, at least 70%, at least 80%, at least 90%, at least 95% or more in a target/host tissue or subject compared to an unmodified dsDNA of the same sequence.

### Elements of DNA Constructs

The dsDNA molecules (e.g., TDSCs) or nucleic acids comprising dsDNA described herein contain elements sufficient to deliver an effector sequence to a target cell, tissue or subject. In some embodiments, the effector sequence is a DNA sequence. In some embodiments, the dsDNA molecule drives expression of an effector, e.g., comprises a promoter and a sequence encoding an RNA or a polypeptide, e.g., a therapeutic RNA or polypeptide. In some embodiments, the DNA constructs described herein further contain one or both of: a nuclear targeting sequence and a maintenance sequence. While many of the embodiments herein refer to a TDSC, it is understood that as applicable an embodiment that refers to a TDSC may also apply to a nucleic acid comprising dsDNA.

### Exonuclease-Resistant DNA End Forms

The TDSCs or nucleic acids comprising dsDNA described herein comprise a DNA end form at each end of the double-stranded DNA molecule. The DNA end forms described herein can, in some instances, comprise a closed end, wherein every nucleotide of the DNA end form is covalently attached to two other nucleotides of the DNA end form. In other instances, the DNA end forms described herein comprise an open end comprising at least one nucleotide that is only covalently attached to one other nucleotide of the DNA end form. The DNA end forms are generally exonuclease resistant. In some instances, a DNA end form comprising a closed end (e.g., a covalently closed end) is resistant to the exonuclease assay described in Example 2. In some instances, a DNA end form comprising an open end (e.g., such as a Y adaptor, blunt end, or sticky end, e.g., as described herein) is resistant to the exonuclease assay described in Example 3.

### Hairpins

In some embodiments, an exonuclease-resistant DNA end form comprises a DNA hairpin. A hairpin generally comprises a single-stranded loop region covalently attached at both the 5′ and 3′ ends to a double-stranded stalk region. In certain embodiments, the single-stranded loop region comprises one or more nucleotides (e.g., 1-2, 2-5, 5-10, 10-15, 15-20, 20-25, 25-30, 30-35, or 35-40 nucleotides) that are not hybridized to another nucleotide. Exemplary hairpin structures, and exemplary dsDNA molecules comprising hairpins, are shown in FIG. 1A.

In certain embodiments, the single-stranded loop region comprises one or more functional elements (e.g., a nuclear import sequence (e.g., a CT3 ssDNA sequence), or a regulatory sequence. In embodiments, a functional element comprised in the single-stranded loop region is heterologous to one or more other elements of the DNA end form and/or a dsDNA molecule comprising the DNA end form. In certain embodiments, the single-stranded loop region of a hairpin loop is less than about 5, 10, 15, 20, 25, 26, 27, 28, 29, or 30 nucleotides in length.

In embodiments, the hairpin is comprised in a dsDNA molecule having a doggybone conformation. In embodiments, the hairpin comprises a protelomerase sequence (e.g., as described herein). In embodiments, the protelomerase sequence is produced by TelN protelomerase, ResT protelomerase, Tel PY54 protelomerase, or TelK protelomerase digestion. In embodiments, the protelomerase sequence is less than about 15, 20, 25, 26, 27, 28, 29, or 30 nucleotides in length. In embodiments, the protelomerase sequences are between about 28 (e.g., 25, 26, 27, 28, 29, 30, 31, 32, 33, 34, or 35) nucleotides and about 56 (e.g., 50, 51, 52, 53, 54, 55, 56, 57, 58, 59, or 60) nucleotides in length. In embodiments, the protelomerase sequences are greater than about 56 (e.g., greater than 50, 51, 52, 53, 54, 55, 56, 57, 58, 59, 60, 65, 70, 75, 80, 90, or 100) nucleotides in length.

A hairpin can be attached to one or both ends of a double-stranded DNA molecule, for example, by ligation (e.g., as described herein). In some embodiments, a dsDNA molecule as described herein comprises, at one or both ends, a DNA hairpin loop. In some embodiments, the upstream exonuclease-resistant DNA end form of a dsDNA molecule as described herein comprises a DNA hairpin loop. In some embodiments, the downstream exonuclease-resistant DNA end form of a dsDNA molecule as described herein comprises a DNA hairpin loop.

In certain embodiments, a DNA hairpin loop comprises one or more unmodified nucleotides. In embodiments, a DNA hairpin loop consists entirely of unmodified nucleotides. In certain embodiments, a DNA hairpin loop comprises one or more chemically modified nucleotides (e.g., phosphorothioate-modified nucleotides, e.g., as described herein). In embodiments, a DNA hairpin loop consists entirely of chemically modified nucleotides (e.g., phosphorothioate-modified nucleotides, e.g., as described herein).

In certain embodiments, the single-stranded loop region of a DNA hairpin loop comprises one or more chemically modified nucleotides (e.g., phosphorothioate-modified nucleotides, e.g., as described herein). In embodiments, at least 50%, at least 60%, at least 70%, at least 75%, at least 80%, at least 85%, at least 90%, at least 95%, at least 96%, at least 97%, or at least 99% of the nucleotides in the single-stranded loop region are chemically modified nucleotides (e.g., phosphorothioate-modified nucleotides, e.g., as described herein). In embodiments, the single-stranded loop region of a DNA hairpin loop consists entirely of chemically modified nucleotides (e.g., phosphorothioate-modified nucleotides, e.g., as described herein). In certain embodiments, the single-stranded loop region of a DNA hairpin loop comprises one or more unmodified nucleotides. In embodiments, the single-stranded loop region of a DNA hairpin loop consists entirely of unmodified nucleotides.

In certain embodiments, the double-stranded stalk region of a DNA hairpin loop comprises one or more unmodified nucleotides. In embodiments, the double-stranded stalk region of a DNA hairpin loop consists entirely of unmodified nucleotides. In certain embodiments, the double-stranded stalk region of a DNA hairpin loop comprises one or more chemically modified nucleotides (e.g., phosphorothioate-modified nucleotides, e.g., as described herein). In embodiments, at least 50%, at least 60%, at least 70%, at least 75%, at least 80%, at least 85%, at least 90%, at least 95%, at least 96%, at least 97%, or at least 99% of the nucleotides in the double-stranded stalk region are modified nucleotides (e.g., phosphorothioate-modified nucleotides, e.g., as described herein). In embodiments, the double-stranded stalk region of a DNA hairpin loop consists entirely of chemically modified nucleotides (e.g., phosphorothioate-modified nucleotides, e.g., as described herein).

In embodiments, the single-stranded loop region of a DNA hairpin loop comprises one or more chemically modified nucleotides (e.g., phosphorothioate-modified nucleotides, e.g., as described herein) and the double-stranded stalk region comprises one or more unmodified nucleotides. In embodiments, at least 50%, at least 60%, at least 70%, at least 75%, at least 80%, at least 85%, at least 90%, at least 95%, at least 96%, at least 97%, or at least 99% of the nucleotides in the single-stranded loop region are chemically modified nucleotides (e.g., phosphorothioate-modified nucleotides, e.g., as described herein). In embodiments, the single-stranded loop region of a DNA hairpin loop consists entirely of chemically modified nucleotides (e.g., phosphorothioate-modified nucleotides, e.g., as described herein) and the double-stranded stalk region consists entirely of unmodified nucleotides.

### Y-Adaptors

In some embodiments, an exonuclease-resistant DNA end form as described herein comprises a Y-adaptor. As described herein, a Y-adaptor generally comprises a pair of single-stranded DNA regions, each attached at one end to a strand of a double-stranded DNA region, thereby forming a “Y” shape (wherein the base of the “Y” represents the double-stranded DNA region, and each of the upper prongs of the “Y” represents the two single-stranded DNA region). Exemplary Y-adaptor structures and exemplary dsDNA molecules comprising Y-adaptors are shown in FIG. 2.

In some embodiments, a Y-adaptor is produced by attaching a hairpin loop comprising a single-stranded region comprising a cleavable moiety to the end of a double-stranded DNA region (e.g., via ligation). The cleavable moiety can then be cleaved to produce the two single-stranded DNA regions of the Y-adaptor.

In certain embodiments, a single-stranded DNA region (e.g., one or both single-stranded DNA regions) of a Y-adaptor comprises one or more chemically modified nucleotides (e.g., phosphorothioate-modified nucleotides, e.g., as described herein). In embodiments, at least 50%, at least 60%, at least 70%, at least 75%, at least 80%, at least 85%, at least 90%, at least 95%, at least 96%, at least 97%, or at least 99% of the nucleotides in the single-stranded DNA region are chemically modified nucleotides (e.g., phosphorothioate-modified nucleotides, e.g., as described herein). In embodiments, a single-stranded DNA region (e.g., one or both single-stranded DNA regions) of a Y-adaptor consists entirely of chemically modified nucleotides (e.g., phosphorothioate-modified nucleotides, e.g., as described herein). In certain embodiments, a single-stranded DNA region (e.g., one or both single-stranded DNA regions) of a Y-adaptor comprises one or more unmodified nucleotides.

In embodiments, a single-stranded DNA region (e.g., one or both single-stranded DNA regions) of a Y-adaptor comprises one or more chemically modified nucleotides (e.g., phosphorothioate-modified nucleotides, e.g., as described herein) and a double-stranded DNA region of the Y-adaptor comprises one or more unmodified nucleotides. In embodiments, at least 50%, at least 60%, at least 70%, at least 75%, at least 80%, at least 85%, at least 90%, at least 95%, at least 96%, at least 97%, or at least 99% of the nucleotides in the single-stranded DNA region or regions are chemically modified nucleotides (e.g., phosphorothioate-modified nucleotides, e.g., as described herein). In embodiments, a single-stranded DNA region (e.g., one or both single-stranded DNA regions) of a Y-adaptor consists entirely of chemically modified nucleotides (e.g., phosphorothioate-modified nucleotides, e.g., as described herein) and the double-stranded DNA region of the Y-adaptor consists entirely of unmodified nucleotides.

### No Loop Closed DNA End Forms

In some embodiments, a dsDNA molecule (e.g., a TDSC) as described herein comprises an exonuclease-resistant DNA end form that is covalently closed but does not include a hairpin loop. For example, in certain embodiments, every nucleotide of a covalently-closed DNA end form is hybridized to another nucleotide. In certain embodiments, the covalently-closed DNA end form comprises a first region and a second region, wherein the first region is capable of hybridizing in its entirety to the second region (e.g., wherein the first region is complementary to the second region) and wherein the 3′ end of the first region is covalently attached to the 5′ end of the second region. In embodiments, a covalently-closed DNA end form as described herein can be attached to one end of a dsDNA molecule as described herein, e.g., by ligation.

### Open DNA End Forms

In some embodiments, a dsDNA molecule (e.g., a TDSC) as described herein comprises an exonuclease-resistant DNA end form that is not covalently closed. In certain embodiments, the DNA end form comprises a blunt end (e.g., a blunt end comprising one or more chemical modifications as described herein) or a sticky end (e.g., a sticky end comprising one or more chemical modifications as described herein).

In certain embodiments, the open DNA end form is produced by nuclease digestion of a covalently closed DNA end form, such as a DNA hairpin. In embodiments, the DNA hairpin comprises a double-stranded stalk region comprising a cleavable moiety on each strand, and the DNA hairpin is then contacted with an enzyme capable of cleaving the cleavable moieties. In embodiments, this results in the formation of a sticky end comprising an overhang. In embodiments, the overhang is digested with an enzyme (e.g., a single-stranded specific nuclease, e.g., a Mung Bean nuclease) to form a blunt end.

In certain embodiments, a DNA end form comprising a blunt end comprises one or more chemically modified nucleotides (e.g., phosphorothioate-modified nucleotides, e.g., as described herein). In embodiments, at least 50%, at least 60%, at least 70%, at least 75%, at least 80%, at least 85%, at least 90%, at least 95%, at least 96%, at least 97%, or at least 99% of the nucleotides in the DNA end form comprising a blunt end are chemically modified nucleotides (e.g., phosphorothioate-modified nucleotides, e.g., as described herein). In embodiments, the DNA end form comprising a blunt end consists entirely of chemically modified nucleotides (e.g., phosphorothioate-modified nucleotides, e.g., as described herein). In embodiments, the terminal base pair of the DNA end form comprising a blunt end comprises a chemically modified nucleotide (e.g., one or both nucleotides of the base pair are chemically modified), e.g., a phosphorothioate-modified nucleotide, e.g., as described herein. In embodiments, a plurality of base pairs (e.g., 2, 3, 4, 5, or 6 base pairs) at the terminal end of the DNA end form comprise chemically modified nucleotides (e.g., one or both nucleotides of the base pair are chemically modified), e.g., phosphorothioate-modified nucleotides, e.g., as described herein. In an embodiment, the three base pairs at the terminal end of the DNA end form comprise chemically modified nucleotides (e.g., one or both nucleotides of the base pair are chemically modified), e.g., phosphorothioate-modified nucleotides, e.g., as described herein. In an embodiment, the six base pairs at the terminal end of the DNA end form comprise chemically modified nucleotides (e.g., one or both nucleotides of the base pair are chemically modified), e.g., phosphorothioate-modified nucleotides, e.g., as described herein.

In certain embodiments, a DNA end form comprising a sticky end comprises one or more chemically modified nucleotides (e.g., phosphorothioate-modified nucleotides, e.g., as described herein). In embodiments, at least 50%, at least 60%, at least 70%, at least 75%, at least 80%, at least 85%, at least 90%, at least 95%, at least 96%, at least 97%, or at least 99% of the nucleotides in the DNA end form comprising a sticky end are chemically modified nucleotides (e.g., phosphorothioate-modified nucleotides, e.g., as described herein). In embodiments, the DNA end form comprising a sticky end consists entirely of chemically modified nucleotides (e.g., phosphorothioate-modified nucleotides, e.g., as described herein). In embodiments, a terminal nucleotide of the DNA end form comprising a sticky end comprises a chemically modified nucleotide (e.g., one or both nucleotides of the base pair are chemically modified), e.g., a phosphorothioate-modified nucleotide, e.g., as described herein. In embodiments, the overhang region of the sticky end of a DNA end form comprises one or more chemically modified nucleotide, e.g., phosphorothioate-modified nucleotides, e.g., as described herein.

### Inverted Terminal Repeats (ITRs)

In some embodiments, a dsDNA molecule (e.g., a TDSC) as described herein comprises an exonuclease-resistant DNA end form comprising an inverted terminal repeat (ITR). In some embodiments, the ITR is an ITR from a virus, e.g., an adenovirus or an adeno-associated virus (AAV). In some embodiments, the ITR comprises a nucleic acid sequence having at least 75%, at least 80%, at least 85%, at least 90%, at least 95%, at least 96%, at least 97%, at least 98%, at least 99%, or 100% sequence identity to an ITR sequence from a virus, e.g., an adenovirus or an adeno-associated virus (AAV). In certain embodiments, the ITR comprises an origin of replication (e.g., a viral origin of replication). In embodiments, a dsDNA molecule as described herein comprises an exonuclease-resistant DNA end form comprising an ITR (e.g., as described herein) at each end. In some embodiments, a dsDNA molecule does not comprise an ITR.

### Promoters and Other Regulatory Sequences

The TDSC or nucleic acid comprising dsDNA described herein may contain a promoter (a DNA sequence at which RNA polymerase and transcription factors bind to, directly or indirectly, to initiate transcription) operably linked to an effector sequence. A promoter may be found in nature operably linked to the effector sequence, or may be heterologous to the effector sequence. A promoter described herein may be native to the target cell or tissue, or heterologous to the target cell or tissue. A promoter may be constitutive, inducible and/or tissue-specific.

Examples of constitutive promoters include the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985), the SV40 promoter, the dihydrofolate reductase promoter, the beta-actin promoter, the phosphoglycerol kinase (PGK) promoter, and the EFlalpha promoter.

Inducible promoters allow regulation of expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of sources. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268:1766-1769 (1995), see also Harvey et al, Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)).

In some embodiments, the native promoter for the sequence encoding the effector can be used.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are known in the art. Exemplary tissue-specific regulatory sequences include, but are not limited to the following tissue specific promoters: a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a alpha-myosin heavy chain (a-MHC) promoter, or a cardiac Troponin T (cTnT) promoter. Other exemplary promoters include Beta-actin promoter, hepatitis B virus core promoter, Sandig et al., Gene Ther., 3:1002-9 (1996); alpha-fetoprotein (AFP) promoter, Arbuthnot et al., Hum. Gene Ther., 7:1503-14 (1996)), bone osteocalcin promoter (Stein et al., Mol. Biol. Rep., 24:185-96 (1997)); bone sialoprotein promoter (Chen et al., J. Bone Miner. Res., 11:654-64 (1996)), CD2 promoter (Hansal et al., J. Immunol., 161:1063-8 (1998); immunoglobulin heavy chain promoter; T cell receptoralpha.-chain promoter, neuronal such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific vgf gene promoter (Piccioli et al., Neuron, 15:373-84 (1995)), among others which will be known to the skilled artisan.

Examples of tissue/cell specific promoters are listed in Table 1:

TABLE 1
Tissue or cell specific promoters

| | | Accession Number; Human |
|---|---|---|
| Tissue/Cell | Promoter | Genome Coordinate (hg38) |
| Skeletal muscle | ACTA1 | NM_001100; chr1: 229,439,090- |
| | | 229,432,090 |
| Melanoma | TYR | NM_000372; chr11: 89,300,750- |
| | | 89,293,750 |
| Hepatoma | a-fetoprotein | NM_001354717; chr4: 73,461,175- |
| | | 73,454,175 |
| Mammary | Mucin 1 | NM_001371720; |
| carcinoma | | chr1: 155,197,900-155,190,900 |
| Prostate Cancer | KLK3 | NM_001648; chr19: 50,865,760- |
| | | 50,858,760 |
| Neuronal cells | ENO2 | NM_001975; chr12: 6,928,700- |
| | | 6,921,700 |
| Response to | HIF-1alpha | NM_001530; chr14: 61,753,200- |
| Hypoxia | | 61,746,200 |
| Retinoblastoma | E2F1 | NM_005225; chr20: 33,691,380- |
| | | 33,684,380 |
| Ionizing radiation | EGR-1 | NM_001964; chr5: 138,474,303- |
| | | 138,467,303 |
| Oncogene | ErbB2 | NM_004448; chr17: 39,735,530- |
| | | 39,728,530 |
| Endothelial cells | vWF | NM_000552; chr12: 6,129,670- |
| | | 6,122,670 |
| Endothelial cells | FLT-1 | NM_002019; chr13: 28,500,100- |
| | | 28,493,100 |
| Endothelial cells | ICAM-2 | NM_001099786; |
| | | chr17: 64,025,630-64,018,630 |
| Retinal pigment | VMD2 | NM_004183; chr11: 61,972,630- |
| epithelium | | 61,965,630 |
| Rod cells | RHO | NM_000539; chr3: 129,540,350- |
| | | 129,533,350 |
| Cone cells | Red/green | NM_020061; chrX: 154,164,030- |
| | opsin | 154,157,030 |
| | (OPN1LW) | |
| Ganglion cells | Thymocyte | NM_006288; chr11: 119,428,150- |
| | antigen | 119,421,150 |
| | (Thy1) | |
| T cells | TIM3 | NM_032782; chr5: 157,114,050- |
| | | 157,107,050 |
| T cells | FOXP3 | NM_014009; chrX: 49,269,700- |
| | | 49,262,700 |
| PBMCs | Vβ6.7 | ENST00000390373.2; |
| | | chr7: 142,493,295-142,486,295 |
| Cell cycle | Cdk1 | NM_001786; chr10: 60,799,850- |
| | | 60,792,850 |

The constructs described herein may also include other native or heterologous expression control elements, such as enhancer elements, polyadenylation sites or Kozak consensus sequences.

### Effector Sequence

The effector sequence of a dsDNA molecule (e.g., TDSC) described herein may be, e.g., a functional DNA sequence, e.g., a therapeutically functional DNA sequence; a DNA sequence encoding a therapeutic peptide, polypeptide or protein; or a DNA sequence encoding a therapeutic RNA (e.g., a non-coding RNA). In some embodiments, a therapeutic payload sequence is an effector sequence described herein.

A therapeutic payload sequence may be used to express the therapeutic payload encoded by the therapeutic payload sequence. An effector sequence may be used to express the effector encoded by the effector sequence. In some embodiments, a therapeutic payload is an effector described herein.

### DNA Effectors:

A therapeutically functional DNA sequence may be a DNA sequence that forms a functional structure, e.g., a DNA sequence comprising a DNA aptamer, DNAzyme or allele-specific oligonucleotide (a DNA ASO). A therapeutically functional DNA sequence may not have a promoter operably linked. In embodiments, a dsDNA molecule (e.g., TDSC) described herein may include one or a plurality of functional DNA sequences, e.g., 2, 3, 4, 5, 6, or more sequences, which may be the same or different.

### Polypeptide Effectors:

A DNA sequence encoding a therapeutic polypeptide may be a DNA sequence encoding one or more effector which is a peptide, protein, or combinations thereof. For example, the DNA sequence encodes an mRNA. The peptide or protein may be: a DNA binding protein; an RNA binding protein; a transporter; a transcription factor; a translation factor; a ribosomal protein; a chromatin remodeling factor; an epigenetic modifying factor; an antigen; a hormone; an enzyme (such as a nuclease, e.g., an endonuclease, e.g., a nuclease element of a CRISPR system, e.g., a Cas9, dCas9, aCas9-nickase, Cpf/Cas12a); a Crispr-linked enzyme, e.g. a base editor or prime editor; a mobile genetic element protein (e.g., a transposase, a retrotransposase, a recombinase, an integrase); a gene writer; a polymerase; a methylase; a demethylase; an acetylase; a deacetylase; a kinase; a phosphatase; a ligase; a deubiquitinase; a protease; an integrase; a recombinase; a topoisomerase; a gyrase; a helicase; a lysosomal acid hydrolase); an antibody (e.g., an intact antibody, a fragment thereof, or a nanobody); a signaling peptide; a receptor ligand; a receptor; a clotting factor; a coagulation factor; a structural protein; a caspase; a membrane protein; a mitochondrial protein; a nuclear protein; an engineered binder such as a centyrin, darpin, or adnectin. See, e.g., Gebauer & Skerra. 2020. Annual Review of Pharmacology and Toxicology 60:1, 391-415.

In embodiments, a dsDNA molecule (e.g., TDSC) described herein may include one or a plurality of sequences encoding a polypeptide, e.g., 2, 3, 4, 5, 6, or more sequences encoding a polypeptide. Each of the plurality may encode the same or different protein. For example, a dsDNA molecule (e.g., TDSC) described herein may include multiple sequences encoding multiple proteins, e.g., a plurality of proteins in a biological pathway.

In some embodiments, a dsDNA molecule (e.g., TDSC) may include a plurality of sequences encoding a polypeptide, e.g., 2, 3, 4, 5, 6, or more sequences encoding a polypeptide, separated by a self-cleaving peptide, e.g., P2A, T2A, E2A or F2A. self-cleaving peptides are 18-22 amino acids long, and can induce ribosomal skipping during protein translation so that two polypeptides can be encoded in the same transcript. Each of the polypeptides may encode the same or different protein. In one embodiment, a dsDNA molecule (e.g., TDSC) may include a promoter followed by a sequence encoding a first polypeptide of interest, a sequence encoding a 2A self-cleaving peptide, a sequence encoding a second polypeptide of interest, and a polyA site. In another embodiment, a dsDNA molecule (e.g., TDSC) may include a promoter followed by a sequence encoding the first polypeptide of interest, a first 2A self-cleaving peptide, a second polypeptide of interest, a sequence encoding a second 2A self-cleaving peptide, a sequence encoding a third polypeptide of interest, and a polyA site.

In some embodiments, the effector comprises a cell penetrating polypeptide. In some embodiments, the effector is a fusion protein that comprises a cell penetrating polypeptide and a second amino acid sequence.

### RNA Effectors:

An effector sequence may be a DNA sequence encoding a non-coding RNA, e.g., one or more of a short interfering RNA (siRNA), a microRNA (miRNA), long non-coding RNA, a piwi-interacting RNA (piRNA), a small nucleolar RNA (snoRNA), a small Cajal body-specific RNA (scaRNA), a transfer RNA (tRNA), a ribosomal RNA (rRNA), an RNA aptamer, and a small nuclear RNA (snRNA).

In some embodiments, the dsDNA molecule (e.g., TDSC) disclosed herein comprises one or more expression sequences that encode a regulatory RNA, e.g., an RNA that modifies expression of an endogenous gene and/or an exogenous gene. In some embodiments, the dsDNA molecule or sequence disclosed herein can comprise a sequence that is antisense to a regulatory nucleic acid like a non-coding RNA, such as, but not limited to, tRNA, lncRNA, miRNA, rRNA, snRNA, microRNA, siRNA, piRNA, snoRNA, snRNA, exRNA, scaRNA, Y RNA, and hnRNA.

In one embodiment, the regulatory nucleic acid targets a host gene. A regulatory nucleic acid may include, but is not limited to, a nucleic acid that hybridizes to an endogenous gene, e.g., an antisense RNA, a guide RNA, a nucleic acid that hybridizes to an exogenous nucleic acid such as a viral DNA or RNA, nucleic acid that hybridizes to an RNA, nucleic acid that interferes with gene transcription, nucleic acid that interferes with RNA translation, nucleic acid that stabilizes RNA or destabilizes RNA such as through targeting for degradation, and nucleic acid that modulates a DNA or RNA binding factor. In one embodiment, the sequence is an miRNA. In some embodiments, the regulatory nucleic acid targets a sense strand of a host gene. In some embodiments, the regulatory nucleic acid targets an antisense strand of a host gene.

In some embodiments, the dsDNA molecule encodes a guide RNA. Guide RNA sequences are generally designed to have a sequence having a length of between 15-30 nucleotides (e.g., 17, 19, 20, 21, 24 nucleotides) that is complementary to the targeted nucleic acid sequence, and a region that facilitates complex formation (e.g., with a tracrRNA or a nuclease). Custom gRNA generators and algorithms are available commercially for use in the design of effective guide RNAs. Gene editing has also been achieved using a chimeric “single guide RNA” (“sgRNA”), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing). Chemically modified sgRNAs have also been demonstrated to be effective in genome editing; see, for example, Hendel et al. (2015) Nature Biotechnol., 985-991. The gRNA may recognize specific DNA sequences (e.g., sequences adjacent to or within a promoter, enhancer, silencer, or repressor of a gene). In one embodiment, the gRNA is used as part of a CRISPR system for gene editing. For the purposes of gene editing, the dsDNA molecule or sequence disclosed herein may be designed to include one or multiple sequences encoding guide RNA sequences corresponding to a desired target DNA sequence; see, for example, Cong et al. (2013) Science, 339:819-823; Ran et al. (2013) Nature Protocols, 8:2281-2308.

A dsDNA molecule or sequence disclosed herein may encode certain regulatory nucleic acids that can inhibit gene expression through the biological process of RNA interference (RNAi). RNAi molecules comprise RNA or RNA-like structures typically containing 15-50 base pairs (such as about 18-25 base pairs) and having a nucleobase sequence identical (complementary) or nearly identical (substantially complementary) to a coding sequence in an expressed target gene within the cell. Such RNAi molecules include, but are not limited to: short interfering RNAs (siRNAs), double-strand RNAs (dsRNA), micro RNAs (miRNAs), short hairpin RNAs (shRNA), meroduplexes, and dicer substrates (U.S. Pat. Nos. 8,084,599 8,349,809 and 8,513,207), RNA antisense oligonucleotides (RNA ASOs).

In one embodiment, the dsDNA molecule or sequence disclosed herein comprises a sequence comprising a sense strand of a lncRNA. In one embodiment, the dsDNA molecule or sequence disclosed herein comprises a sequence encoding an antisense strand of a lncRNA.

The dsDNA molecule or sequence disclosed herein may encode a regulatory nucleic acid substantially complementary, or fully complementary, to a fragment of an endogenous gene or gene product (e.g., mRNA). The regulatory nucleic acids may complement sequences at the boundary between introns and exons, in between exons, or adjacent to exon, to prevent the maturation of newly-generated nuclear RNA transcripts of specific genes into mRNA for transcription. The regulatory nucleic acids that are complementary to specific genes can hybridize with the mRNA for that gene and prevent its translation. The antisense regulatory nucleic acid can be DNA, RNA, or a derivative or hybrid thereof. In some embodiments, the regulatory nucleic acid comprises a protein-binding site that can bind to a protein that participates in regulation of expression of an endogenous gene or an exogenous gene.

The length of a dsDNA molecule or sequence disclosed herein that may encode a regulatory nucleic acid that hybridizes to a transcript of interest and may be, for instance, between about 5 to 30 nucleotides, between about 10 to 30 nucleotides, or about 11, 12, 13, 14, 15, 16, 17, 18, 19, 20, 21, 22, 23, 24, 25, 26, 27, 28, 29, 30 or more nucleotides. The degree of identity of the regulatory nucleic acid to the targeted transcript should be at least 75%, at least 80%, at least 85%, at least 90%, or at least 95%.

A dsDNA molecule or sequence disclosed herein may encode a micro-RNA (miRNA) molecule identical to about 5 to about 30 contiguous nucleotides of a target gene. In some embodiments, the miRNA sequence targets a mRNA and commences with the dinucleotide AA, comprises a GC-content of about 30-70% (about 30-60%, about 40-60%, or about 45%-55%), and does not have a high percentage identity to any nucleotide sequence other than the target in the genome of the mammal in which it is to be introduced, for example as determined by standard BLAST search. In some embodiments, the dsDNA molecule or sequence disclosed herein encodes at least one miRNA, e.g., 2, 3, 4, 5, 6, or more. In some embodiments, the dsDNA molecule or sequence disclosed herein comprises a sequence that encodes an miRNA having at least about 75%, at least about 80%, at least about 85%, at least about 90%, at least about 95%, at least about 96%, at least about 97%, at least about 98%, at least about 99% or 100% nucleotide sequence identity to any one of the nucleotide sequences or a sequence that is complementary to a target sequence. Lists of known miRNA sequences can be found in databases maintained by research organizations, such as Wellcome Trust Sanger Institute, Penn Center for Bioinformatics, Memorial Sloan Kettering Cancer Center, and European Molecule Biology Laboratory, among others. Known effective siRNA sequences and cognate binding sites are also well represented in the relevant literature. RNAi molecules are readily designed by technologies known in the art. In addition, there are computational tools that increase the chance of finding effective and specific sequence motifs (see, e.g., Lagana et al., Methods Mol. Bio., 2015, 1269:393-412).

The dsDNA molecule or sequence disclosed herein may modulate expression of RNA encoded by a gene. Because multiple genes can share some degree of sequence homology with each other, in some embodiments, the dsDNA molecule or sequence disclosed herein can be designed to target a class of genes with sufficient sequence homology. In some embodiments, the dsDNA molecule or sequence disclosed herein can contain a sequence that has complementarity to sequences that are shared amongst different gene targets or are unique for a specific gene target. In some embodiments, the dsDNA molecule or sequence disclosed herein can be designed to target conserved regions of an RNA sequence having homology between several genes thereby targeting several genes in a gene family (e.g., different gene isoforms, splice variants, mutant genes, etc.). In some embodiments, the dsDNA molecule or sequence disclosed herein can be designed to target a sequence that is unique to a specific RNA sequence of a single gene.

In embodiments, the effector sequence encoding a regulatory RNA has a length less than 5000 bps (e.g., less than about 5000 bps, less than about 4000 bps, less than about 3000 bps, less than about 2000 bps, less than about 1000 bps, less than about 900 bps, less than about 800 bps, less than about 700 bps, less than about 600 bps, less than about 500 bps, less than about 400 bps, less than about 300 bps, less than about 200 bps, less than about 100 bps, less than about 50 bps, less than about 40 bps, less than about 30 bps, less than about 20 bps, less than about 10 bps, or less). In some embodiments, the effector sequence has, independently or in addition to, a length greater than 10 bps (e.g., at least about 10 bps, at least about 20 bps, at least about 30 bps, at least about 40 bps, at least about 50 bps, at least about 60 bps, at least about 70 bps, at least about 80 bps, at least about 90 bps, at least about 100 bps, at least about 200 bps, at least about 300 bps, at least about 400 bps, at least about 500 bps, at least about 600 bps, at least about 700 bps, at least about 800 bps, at least about 900 bps, at least about 1000 kb, at least about 1.1 kb, at least about 1.2 kb, at least about 1.3 kb, at least about 1.4 kb, at least about 1.5 kb, at least about 1.6 kb, at least about 1.7 kb, at least about 1.8 kb, at least about 1.9 kb, at least about 2 kb, at least about 2.1 kb, at least about 2.2 kb, at least about 2.3 kb, at least about 2.4 kb, at least about 2.5 kb, at least about 2.6 kb, at least about 2.7 kb, at least about 2.8 kb, at least about 2.9 kb, at least about 3 kb, at least about 3.1 kb, at least about 3.2 kb, at least about 3.3 kb, at least about 3.4 kb, at least about 3.5 kb, at least about 3.6 kb, at least about 3.7 kb, at least about 3.8 kb, at least about 3.9 kb, at least about 4 kb, at least about 4.1 kb, at least about 4.2 kb, at least about 4.3 kb, at least about 4.4 kb, at least about 4.5 kb, at least about 4.6 kb, at least about 4.7 kb, at least about 4.8 kb, at least about 4.9 kb, at least about 5 kb or greater).

In some embodiments, a dsDNA molecule or sequence disclosed herein comprises one or more of the features described hereinabove, e.g., one or more structural DNA sequence, a sequence encoding one or more peptides or proteins, a sequence encoding one or more regulatory element, a sequence encoding one or more regulatory nucleic acids, e.g., one or more non-coding RNAs, other expression sequences, and any combination of the aforementioned. A construct described herein may have one or a plurality of effector sequences, e.g., 2, 3, 4, 5 or more effector sequences. In the case of a plurality of effector sequences in a single construct, the effector sequences may be the same or different.

In one embodiment, the dsDNA molecule includes a therapeutically functional, structural DNA sequence. In one embodiment, the dsDNA molecule includes a promoter and a sequence encoding a therapeutic peptide, polypeptide, or protein described herein. In one embodiment, the dsDNA molecule includes a promoter and a sequence encoding a regulatory RNA described herein.

In some embodiments, the effector sequence that encodes a polypeptide or protein is codon optimized, e.g., codon optimized for expression in a mammal, e.g., a human. In general, codon optimization means modifying a nucleic acid sequence for enhanced expression in the host cells of interest by replacing at least one codon (e.g., one or more, e.g., 1, 2, 3, 4, 5, 10, 15, 20, 25, 50, or more codons; e.g., at least 1%, at least 5%, at least 10%, at least 20%, at least 25%, at least 50%, at least 60%, at least 70%, at least 80%, at least 90% or 100%) of the native sequence with codons that are more frequently or most frequently used in the genes of that host cell while maintaining the native amino acid sequence. Codon usage tables are available, for example, at the “Codon Usage Database” available at http://www.kazusa.or.jp/codon/. These tables can be adapted in a number of ways, see, e.g., Nakamura et al., 2000, Nucl. Acids Res. 28:292. Computer algorithms for codon optimizing a particular sequence for expression in a particular host cell are also available, such as Gene Forge.

### Nuclear Targeting Sequences (NTS)

A dsDNA molecule (e.g. TDSC) or nucleic acid comprising dsDNA (e.g., as disclosed herein) may include a nuclear targeting sequence (NTS) that facilitates transport of DNA from the cytoplasm into the nucleus of a cell. An NTS includes binding sites to proteins (e.g., transcription factors, chaperones, etc.) which bind to importin which transports cargo into the nucleus via the nuclear pore complex. In embodiments, an NTS may function generally (e.g. SV40 enhancer NTS). In other embodiments, NTS's may be cell or tissue specific, e.g., containing binding sites for transcription factors expressed in unique cell types that may target a dsDNA molecule described herein to the nucleus in a cell-specific manner (e.g., SRF, Nkx3). An NTS can be functional in multiple locations in a dsDNA molecule described herein, e.g., before the promoter and/or after the effector sequence.

An NTS may be viral or non-viral derived. NTSs are described, e.g., in Le Guen et al. 2021. Nucleic Acids Vol. 24: 477-486. Examples of NTS's are disclosed in Table 2:

TABLE 2
Exemplary nuclear targeting sequences

| Viral/ | | |
|---|---|---|
| Non-viral | Name | Sequence |
| Viral | SV40 | 5′-cccaagaagaagaggaaagtc-3′ |
| | | (SEQ ID NO: 1) |
| Non-viral | 3NF | 5′-ctggggactttccagcctggggac |
| | | tttccagctgggactttccagg-3′ |
| | | (SEQ ID NO: 85) |

In some embodiments, the NTS has a sequence according to Table 2, or a functional sequence having at least 80%, at least 90%, at least 95%, at least 96%, at least 97%, at least 98%, or at least 99% identity thereto.

### Nuclear Import Proteins

In some embodiments, a TDSC or nucleic acid comprising dsDNA (e.g., as described herein) is capable of being imported into the nucleus, e.g., by a nuclear import protein. In some embodiments, a TDSC or nucleic acid comprising dsDNA (e.g., as described herein) can be bound by a nuclear import protein. In some embodiments, a TDSC or nucleic acid comprising dsDNA (e.g., as described herein) comprises a recognition sequence for a nuclear import protein. In some embodiments, an exonuclease-resistant DNA end form (e.g., comprised in a TDSC or nucleic acid comprising dsDNA, e.g., as described herein) comprises a recognition sequence for a nuclear import protein.

Exemplary import proteins include, e.g., basic helix-loop-helix (bHLH) proteins, heterogeneous nuclear ribonucleoprotein (hnRNP) isoforms, or nuclear factor I (NFI) proteins. In some embodiments, the import protein comprises an importin.

In some embodiments, the import protein comprises a Ran binding protein. In some embodiments, the import protein comprises a homeobox transcription factor. In some embodiments, the import factor specifically binds an E-box, a DTS, a promoter, a telomere, an ATTT motif, a cell cycle regulatory unit (CCRU), a CT3 sequence, an S/MAR, a topoisomerase II consensus sequence, an ARS consensus sequence, a 3NF, or a viral ori.

### Maintenance Sequence

A dsDNA molecule (e.g., a TDSC) disclosed herein may include a maintenance sequence that supports or enables sustained gene expression through successive rounds of cell division and/or progenitor differentiation in a host cell for a dsDNA molecule of the invention. In embodiments, a maintenance sequence is a nuclear scaffold/matrix attachment region (S/MAR). S/MAR elements are diverse, AT-rich sequences ranging from 60-500 bp that are conserved across species, thought to anchor chromatin to nuclear matrix proteins during interphase (Bode et al. 2003. Chromosome Res 11, 435-445). An S/MAR can be incorporated into a dsDNA molecule described herein to facilitate long-term transgene expression and extra-chromosomal maintenance. In one embodiment, the maintenance sequence is human interferon-beta MAR (5′tataattcactggaatttttttgtgtgtatggtatgacatatgggttcccttttattttttacatataaatatatttccctgtttttctaaaaaagaaaa agatcatcattttcccattgtaaaatgccatatttttttcataggtcacttacata-3′ (SEQ ID NO: 39)), or a functional sequence having at least 80%, at least 90%, at least 95%, or at least 98% identity thereto. In embodiments, S/MARs useful in the constructs described herein can be found by searching the MARome at http://bioinfo.net.in/MARome, described also by Narwade et al. 2019. Nucleic Acids Research. Volume 47, Issue 14: 7247-7261.

In embodiments, a dsDNA molecule (e.g., a TDSC) described herein is capable of replicating in a mammalian cell, e.g., human cell. In some embodiments, a dsDNA molecule described herein is maintained in a host cell, tissue or subject through at least one cell division. For example, a dsDNA molecule described herein is maintained in a host cell, tissue or subject through at least 2, at least 3, at least 4, at least 5, at least 6, at least 7, at least 8, at least 10, at least 15, at least 20, at least 40, at least 50 or more cell divisions. In vitro, cell division may be tracked by flow cytometry or microscopy. In vivo, cell division may be tracked by intravital microscopy.

### Other Elements

A TDSC or nucleic acid comprising dsDNA disclosed herein may also include other control elements operably linked to the effector sequence, e.g., the sequence encoding an effector, in a manner which permits its transport, localization, transcription, translation and/or expression in a target cell, or which promotes its degradation or repression of expression in a non-target cell. As used herein, “operably linked” sequences include both expression control sequences that are contiguous with the sequence encoding the effector and expression control sequences that act in trans or at a distance to control the sequence encoding the effector. The precise nature of regulatory sequences needed for gene expression in host cells may vary between species, tissues or cell types, but in general may include, as necessary, 5′ non-transcribed and 5′ non-translated sequences involved with the initiation of transcription and translation respectively, such as a TATA box, capping sequence, CAAT sequence, enhancer elements and the like. Regulatory sequences may also include enhancer sequences or upstream activator sequences as desired. The constructs described herein may optionally include 5′ leader or signal sequences.

### Structure of DNA Constructs

In some embodiments, the dsDNA molecule (e.g., TDSC) disclosed herein is at least about 20 nucleotides, at least about 30 nucleotides, at least about 40 nucleotides, at least about 50 nucleotides, at least about 75 nucleotides, at least about 100 nucleotides, at least about 200 nucleotides, at least about 300 nucleotides, at least about 500 nucleotides, at least about 1000 nucleotides, at least about 2000 nucleotides, at least about 3000 nucleotides, at least about 4000 nucleotides, at least about 5000 nucleotides, at least about 6000 nucleotides, at least about 7000 nucleotides, at least about 8000 nucleotides, at least about 9000 nucleotides, at least about 10,000 nucleotides, at least about 20,000 nucleotides, at least about 30,000 nucleotides, at least about 40,000 nucleotides, or at least about 50,000 nucleotides in length. In some embodiments, the dsDNA molecule disclosed herein is between 20-30, 30-40, 40-50, 50-75, 75-100, 100-200, 200-300, 300-500, 500-1000, 1000-2000, 2000-3000, 3000-4000, 4000-5000, 5000-6000, 6000-7000, 7000-8000, 8000-9000, 9000-10,000, 10,000-20,000, 20,000-30,000, 30,000-40,000, or 40,000-50,000 nucleotides in length. In some embodiments, the size of a dsDNA molecule disclosed herein is a length sufficient to encode useful polypeptides or RNAs. It is understood that when the length of a linear closed-ended dsDNA molecule is discussed herein, the length refers to the number of nucleotides starting with and including the upstream end, through the downstream end. For example, a no-loop dsDNA molecule having 100 base pairs would have a length of 100 nucleotides.

In some embodiments, a dsDNA molecule comprises an exonuclease-resistant DNA end form (e.g., as described herein). In some embodiments, the DNA end form is at least 2, at least 3, at least 4, at least 5, at least 10, at least 15, at least 20, at least 25, at least 30, at least 40, at least 50, at least 60, at least 70, at least 80, at least 90, or at least 100 nucleotides in length. In some embodiments, the DNA end form is less than 10, less than 15, less than 20, less than 25, less than 30, less than 40, less than 50, less than 60, less than 70, less than 80, less than 90, or less than 100 nucleotides in length. In some embodiments, the DNA end form is 2-5, 5-10, 10-15, 15-20, 20-25, 25-30, 30-35, 35-40, 40-45, 45-50, 50-55, 55-60, 60-70, 70-80, 80-90, or 90-100 nucleotides in length.

In some embodiments, a dsDNA molecule comprises double stranded region encoding an effector (e.g., a polypeptide or RNA, e.g., as described herein), e.g., positioned between two exonuclease-resistant DNA end forms. In some embodiments, the double stranded region is at least 10, at least 15, at least 20, at least 25, at least 30, at least 40, at least 50, at least 60, at least 70, at least 80, at least 90, at least 100, at least 200, at least 300, at least 400, at least 500, at least 600, at least 700, at least 800, at least 900, at least 1000, at least 2000, at least 3000, at least 4000, at least 5000, at least 6000, at least 7000, at least 8000, at least 9000, at least 10,000, at least 20,000, at least 30,000, at least 40,000, or at least 50,000 nucleotides in length. In some embodiments, the double stranded region is less than 50, less than 60, less than 70, less than 80, less than 90, less than 100, less than 200, less than 300, less than 400, less than 500, less than 600, less than 700, less than 800, less than 900, less than 1000, less than 2000, less than 3000, less than 4000, less than 5000, less than 6000, less than 7000, less than 8000, less than 9000, less than 10,000, less than 20,000, less than 30,000, less than 40,000, or less than 50,000 nucleotides in length. In some embodiments, the double stranded region is 10-15, 15-20, 20-25, 25-30, 30-35, 35-40, 40-45, 45-50, 50-55, 55-60, 60-70, 70-80, 80-90, 90-100, 100-200, 200-300, 300-400, 400-500, 500-600, 600-700, 700-800, 800-900, 900-1000, 1000-2000, 2000-3000, 3000-4000, 4000-5000, 5000-6000, 6000-7000, 7000-8000, 8000-9000, 9000-10,000, 10,000-20,000, 20,000-30,000, 30,000-40,000, or 40,000 to 50,000 nucleotides in length.

A dsDNA molecule described herein may have less than a threshold level of single stranded structures. In one embodiment, the dsDNA molecule does not comprise more than 20, 18, 16, 14, 12, 10, 8, 7, 5, 4, 3, 2, or 1 single stranded region longer than 100, 80, 70, 60, 50, 40, 30, 20 or 10 bases, e.g., does not comprise single stranded regions longer than 100, 80, 70, 60, 50, 40, 30, 20 or 10 bases. In one embodiment, double stranded regions formed by a dsDNA molecule described herein is determined as described by Xayaphoummine et al. 2005. Kinefold web server for RNA/DNA folding path and structure prediction including pseudoknots and knots. Nucleic Acids Research, Volume 33:W605-610. In one embodiment, the Kinefold website (http://kinefold.curie.fr/cgi-bin/form.pl) is used to predict double stranded regions of a construct described herein, using the following parameters:
Sequence to fold: enter and select “DNA sequence”
Stochastic Simulation: Co-transcriptional fold, 3 milliseconds
Simulated molecular time: default
Pseudoknots: not allowed
Entanglements: non crossing
Random seed: 11453

In some embodiments, a dsDNA form described herein is asymmetrically modified, where one strand comprises chemically modified nucleobases and the other strand is substantially free of chemically modified nucleobases. In some embodiments, the hemi-modified DNA may be completely free of chemically modified nucleotides on the antisense strand, and in other embodiments, the hemi-modified DNA may comprise a few chemical modifications (such as backbone modifications, e.g., phosphorothioate) on the antisense strand. In some embodiments, the hemi-modified DNA molecule comprises chemically modified nucleotides (e.g., nucleotides comprising chemically modified nucleobases) on the sense strand. In some embodiments, the hemi-modified DNA molecule comprises chemically modified cytosine nucleotides on the sense strand.

### Production

In some embodiments, a dsDNA molecule (e.g., TDSC) as described herein is produced from a plasmid assembled to contain the desired elements described herein. The plasmid template can be assembled, for example, using Golden Gate cloning for assembly of multiple DNA fragments in a defined linear order in a recipient vector using a one-pot assembly procedure. Golden Gate cloning is described in Marillonnet & Grützner, 2020, Synthetic DNA assembly using golden gate cloning and the hierarchical modular cloning pipeline, Current Protocols in Molecular Biology, 130:e115. In some embodiments, a plasmid template is linearized, for example, by digestion with a nuclease (e.g., a restriction endonuclease) or by PCR amplification of a linear nucleic acid sequence from the plasmid template.

In some embodiments, a dsDNA molecule comprising chemical modifications on one strand is produced by amplification of one strand (e.g., from a plasmid template) using a dNTP mixture comprising one or more chemically modified nucleotides and a primer that can amplify one strand of the dsDNA molecule sequence. In certain embodiments, the opposite strand (e.g., an unmodified strand or a differently chemically modified strand, e.g., as described herein, for example, in FIGS. 1A-2) is produced in a separate amplification reaction, e.g., using a dNTP mixture comprising unmodified nucleotides or a different set of chemically modified nucleotides, and a primer that can amplify the opposite strand of the dsDNA molecule sequence.

In some embodiments, a dsDNA molecule comprising the same chemical modification(s) on both strand is produced by amplification of the dsDNA molecule strands (e.g., from a plasmid template) using a dNTP mixture comprising one or more chemically modified nucleotides and primers that can amplify both strand of the dsDNA molecule sequence.

In some embodiments, an exonuclease-resistant DNA end form (e.g., as described herein) is introduced (e.g., attached) to one or both ends of a dsDNA molecule. In certain embodiments, the DNA end form is attached to an end of the dsDNA molecule by ligation. In embodiments, attachment (e.g., ligation) of the DNA end form (e.g., a covalently closed DNA end form) to the dsDNA molecule produces the final dsDNA molecule. In certain embodiments, exonuclease resistance of the attached DNA end form is confirmed, for example, by incubating the dsDNA molecule in the presence of an exonuclease (e.g., Exonuclease III and/or Mung Bean Nuclease), e.g., as described in Examples 2 and 3. In embodiments, exonuclease resistance of the attached DNA end form is confirmed, for example, by incubating the dsDNA molecule in the presence of Exonuclease III. In embodiments, the DNA end form comprises a blunt end, sticky end, or Y-adaptor (e.g., as described herein), and the exonuclease resistance of the attached DNA end form is confirmed by incubating the dsDNA molecule in the presence of Exonuclease III and (e.g., subsequently, prior to, or concurrently) Mung Bean nuclease.

In certain embodiments, the DNA end form is attached to the end of the dsDNA molecule in a nascent form (e.g., a non-covalently closed DNA end form may be attached to the dsDNA molecule as a hairpin. In a subsequent step, the nascent form of the DNA end form may be further modified (e.g., cleaved) to produce the final DNA end form. For example, a non-covalently closed DNA end form may be produced by cleavage of a nascent form, e.g., by a nuclease. In some embodiments, a nascent form comprising an overhang or sticky end can be converted to a blunt end by digestion with a single strand-specific nuclease, e.g., a Mung Bean nuclease. In some embodiments, a nascent form comprising a hairpin comprising a cleavable moiety in its single-stranded loop region is converted to a Y-adaptor by cleavage of the cleavable moiety.

In an embodiment, the method further comprises formulating the enriched or purified dsDNA molecule for pharmaceutical use, e.g., formulating the dsDNA molecule with a pharmaceutically acceptable excipient and/or with a carrier, e.g., an LNP.

In an embodiment, a method described herein comprises enriching or purifying the dsDNA molecule. In an embodiment, the enriching or purifying includes substantially removing from the dsDNA molecule one or more impurity selected from: endotoxin, mononucleotides, chemically modified mononucleotides, single stranded DNA, DNA fragments or truncations, and proteins (e.g., enzymes, e.g., ligases, restriction enzymes).

The dsDNA molecule may be enriched or purified from impurities or byproducts selected from the group consisting of: endotoxin, mononucleotides, chemically modified mononucleotides, single stranded DNA, circular DNA, proteins (e.g., enzymes, e.g., ligases, restriction enzymes), DNA fragments or truncations. In some embodiments, the purified dsDNA molecule is substantially free of process byproducts and impurities, e.g., process byproducts or impurities described herein.

In embodiments, a pharmaceutical composition comprising a dsDNA molecule described herein is substantially free of impurities or process byproducts, e.g., selected from the group consisting of: endotoxin, mononucleotides, chemically modified mononucleotides, DNA fragments or truncations, and proteins (e.g., enzymes, e.g., ligases, restriction enzymes). In some embodiments, the pharmaceutical composition is substantially free of circular DNA. In some embodiments, the pharmaceutical composition is substantially free of RNA. In some embodiments, the pharmaceutical composition is substantially free of single stranded DNA (ssDNA). In some embodiments, the pharmaceutical composition is substantially free of DNA fragments. In some embodiments, the pharmaceutical composition is substantially free of open-ended double stranded DNA. In some embodiments, the pharmaceutical composition is substantially free of microorganisms. In some embodiments, the pharmaceutical composition is substantially free of bacterial proteins. In some embodiments, the pharmaceutical composition is substantially free of bacterial DNA.

In some embodiments, all dsDNA molecules in the pharmaceutical composition have substantially the same length in nucleotides (e.g., all dsDNA molecules in the pharmaceutical composition have the same length in nucleotides). In some embodiments, at least 50%, at least 60%, at least 70%, at least 80%, or at least 90% of dsDNA molecules in the pharmaceutical composition have the same length in nucleotides. In some embodiments, the therapeutic payload sequences of dsDNA molecules in the pharmaceutical composition have substantially the same length in nucleotides (e.g., the therapeutic payload sequences of dsDNA molecules in the pharmaceutical composition have the same length in nucleotides). In some embodiments, all dsDNA molecules in the pharmaceutical composition have a length of between 100, 200, 500, or 1000 nucleotides of each other. In some embodiments, all dsDNA molecules in the pharmaceutical composition have a length of between 500-1000, 1000-2000, 2000-3000, 3000-4000, 4000-5000, 5000-6000, 6000-7000, 7000-8000, 8000-9000, 9000-10000, 10000-11000, or 11000-12000 nucleotides. In some embodiments, all dsDNA molecules in the pharmaceutical composition encode substantially the same effector (e.g., all dsDNA molecules in the pharmaceutical composition encode the same effector). In some embodiments, all dsDNA molecules in the pharmaceutical composition have substantially the same sequence (e.g., all dsDNA molecules in the pharmaceutical composition have the same sequence).

In some embodiments, the pharmaceutical composition is substantially free of a given impurity or process byproduct, e.g., the pharmaceutical composition is free of the impurity or process byproduct.

In some embodiments, a pharmaceutical composition comprises a plurality of dsDNA molecules described herein, wherein the dsDNA molecules of the plurality comprise an amplicon region beginning at the start codon for the encoded polypeptide of the dsDNA molecules and extending 200 base pairs to 210 base pairs, 210 base pairs to 220 base pairs, 220 base pairs to 230 base pairs, 230 base pairs to 240 base pairs, or 240 base pairs to 250 base pairs in the direction of transcription. In some embodiments, the pharmaceutical composition comprises a first sub-population of dsDNA molecules, wherein each amplicon region in the first sub-population has the same DNA sequence, e.g., a desired sequence, and at least one additional dsDNA molecule, wherein the amplicon region of the additional dsDNA molecule has a different DNA sequence from the amplicon region in the first sub-population, e.g., the amplicon region of the additional dsDNA molecule has one or more errors relative to the desired DNA sequence. In some embodiments, at least 70%, between 20% and 70%, or 20% or less of the dsDNA molecules are part of the first sub-population. In some embodiments, the dsDNA molecules in the pharmaceutical composition have an average of at least 5, between 1 and 5, or less than 1 substitutions per kilobase relative to the desired DNA sequence. In some embodiments, the dsDNA molecules in the pharmaceutical composition have an average of at least 0.1, between 0.05 and 0.1, or less than 0.05 insertions per kilobase relative to the desired DNA sequence. In some embodiments, the dsDNA molecules in the pharmaceutical composition have an average of at least 0.25, between 0.15 and 0.25, or less than 0.15 deletions per kilobase relative to the desired DNA sequence. In some embodiments, in an amplicon region, on average at least 99.5%, between 0.15% and 99.5%, or less than 0.15% of the positions that are adenine, cytosine, guanine, or thymine in the desired DNA sequence are the same nucleobase in the dsDNA molecules in the pharmaceutical composition.

In some embodiments, a pharmaceutical composition comprises a plurality of dsDNA molecules described herein. In some embodiments, when the plurality of dsDNA molecules is introduced into a cell, the cell transcribes the dsDNA molecules to produce a plurality of RNA molecules, the plurality of RNA molecules comprising an amplicon region beginning at the start codon for the encoded polypeptide of the dsDNA molecules and extending at least 200 base pairs, at least 210 base pairs, at least 220 base pairs, at least 230 base pairs, at least 240 base pairs, or at least 250 base pairs in the direction of transcription. In some embodiments, the plurality of RNA molecules comprises a first sub-population of RNA molecules, wherein each amplicon region in the first sub-population has the same RNA sequence, e.g., a desired RNA sequence, and at least one additional RNA molecule, wherein the amplicon region of the additional RNA molecule has a different RNA sequence from the amplicon region in the first sub-population, e.g., wherein the amplicon region of the additional RNA molecule has one or more errors relative to the desired RNA sequence. In some embodiments, at least 70%, between 20% and 70%, or 20% or less of the RNA molecules are part of the first sub-population. In some embodiments, the RNA molecules in the pharmaceutical composition have an average of at least 5, between 1 and 5, or less than 1 substitutions per kilobase relative to the desired RNA sequence. In some embodiments, the RNA molecules in the pharmaceutical composition have an average of at least 0.1, between 0.05 and 0.1, or less than 0.05 insertions per kilobase relative to the desired RNA sequence. In some embodiments, the RNA molecules in the pharmaceutical composition have an average of at least 0.25, between 0.15 and 0.25, or less than 0.15 deletions per kilobase relative to the desired RNA sequence.

In some embodiments, a dsDNA molecule is formulated with a lipid based carrier, e.g., a lipid nanoparticle (LNP), e.g., as described in Example 1.

The dsDNA molecule may be sequenced to confirm the desired, designed sequence. In embodiments, other structural analysis of the dsDNA molecule (e.g., restriction enzyme analysis) may be performed to confirm or verify its sequence.

A chemically modified dsDNA molecule described herein may be produced by a number of methods, including methods routine in the art. For instance, a chemically modified dsDNA molecule can be produced by performing polymerase chain reaction on a DNA template in the presence of unmodified and chemically modified nucleotides and a suitable polymerase. Exemplary suitable polymerases are described in Example 5 and include KOD polymerase (710864, Sigma Aldrich), KOD Xtreme polymerase (719753, Sigma Aldrich), and Deep Vent polymerase (M0258, NEB). A wide variety of other polymerases are available, e.g., from commercial sources. Other polymerases can be used so long as they incorporate chemically modified nucleotides with a sufficiently high efficiency.

A chemically modified dsDNA molecule may also be produced by a method that does not comprise performing polymerase chain reaction. For instance, direct chemical synthesis may be used.

A chemically modified dsDNA molecule may be produced by providing a dsDNA molecule and chemically modifying nucleotides of the dsDNA molecule. For instance, a dsDNA molecule may be contacted with an enzyme, resulting in a chemically modified dsDNA molecule. In some embodiments, the enzyme is an enzyme that chemically modifies cytosine, e.g., a 5mC methyltransferase or a 5-hmC glycosyltransferase (e.g., Zymo Research, E2026). In some embodiments, the enzyme converts an unmodified nucleotide into a chemically modified nucleotide. In some embodiments, the enzyme converts a chemically modified nucleotide into a differently modified nucleotide.

In some embodiments, a dsDNA molecule as described herein, e.g., a dsDNA molecule comprising a chemically modified cytosine nucleotide having a substitution other than hydrogen at carbon 5 of the cytosine, is produced from a plasmid assembled to contain the desired elements described herein. In some embodiments, the plasmid template comprises a promoter sequence (e.g. an Ef1a promoter sequence), an effector sequence (e.g., a sequence encoding a model/marker protein, e.g., a sequence encoding mCherry), an enhancer sequence (e.g., a SV40 enhancer sequence), a maintenance sequence (e.g., a sequence from human interferon-β MAR), and/or a second strand motif (e.g., a sequence from AAV2 wildtype ITR). The plasmid template can be designed using standard DNA design manipulation software. Once the designed, the plasmids can be ordered from a commercial supplier (GenScript). In some embodiments, the plasmid template is used as a template for PCR amplification.

In some embodiments, purification involves reduction, e.g., partial reduction or complete reduction, of one or more contaminants.

### Pharmaceutical Compositions

The present disclosure includes a dsDNA molecule (e.g., TDSC) and related compositions in combination with one or more pharmaceutically acceptable excipients and/or carriers.

Pharmaceutical compositions may optionally comprise one or more additional active substances, e.g., therapeutically and/or prophylactically active substances. Pharmaceutical compositions of the present invention are generally sterile and/or pyrogen-free.

A dsDNA molecule described herein may be formulated without a carrier, e.g., the dsDNA molecule described herein may be administered to a host cell, tissue or subject “naked”. A naked formulation may include pharmaceutical excipients or diluents but lacks a carrier.

Pharmaceutically acceptable excipients or diluents may comprise an inactive substance that serves as a vehicle or medium for the compositions described herein, such as any one of the inactive ingredients approved by the United States Food and Drug Administration (FDA) and listed in the Inactive Ingredient Database, which is incorporated by reference herein. Non-limiting examples of pharmaceutically acceptable excipients or diluents include solvents, aqueous solvents, non-aqueous solvents, tonicity agents, dispersion media, cryoprotectants, diluents, suspension aids, surface active agents, isotonic agents, thickening agents, emulsifying agents, preservatives, hyaluronidases, dispersing agents, preservatives, lubricants, granulating agents, disintegrating agents, binding agents, antioxidants, buffering agents (e.g., phosphate buffered saline (PBS)), lubricating agents, oils, and mixtures thereof.

General considerations in the formulation and/or manufacture of pharmaceutical agents may be found, for example, in Remington: The Science and Practice of Pharmacy 21st ed., Lippincott Williams & Wilkins, 2005 (incorporated herein by reference).

### Carriers

A dsDNA molecule (e.g., TDSC) described herein may also be formulated, or included, with a carrier. General considerations of carriers and delivery of pharmaceutical agents may be found, for example, in Delivery Technologies for Biopharmaceuticals: Peptides, Proteins, Nucleic Acids and Vaccines (Lene Jorgensen and Hanne Morck Nielson, Eds.) Wiley; 1st edition (Dec. 21, 2009); and Vargason et al. 2021. Nat Biomed Eng 5, 951-967.

Non-limiting examples of carriers include carbohydrate carriers (e.g., an anhydride-modified phytoglycogen or glycogen-type material, GalNAc), nanoparticles (e.g., a nanoparticle that encapsulates or is covalently linked to the dsDNA molecule, gold nanoparticles, silica nanoparticles), lipid particles (e.g., liposomes, lipid nanoparticles), cationic carriers (e.g., a cationic lipopolymer or transfection reagent), fusosomes, non-nucleated cells (e.g., ex vivo differentiated reticulocytes), nucleated cells, exosomes, protein carriers (e.g., a protein covalently linked to the dsDNA molecule), peptides (e.g., cell-penetrating peptides), materials (e.g., graphene oxide), single pure lipids (e.g., cholesterol), DNA origami (e.g., DNA tetrahedron).

In one embodiment, the dsDNA molecule compositions, constructs and systems described herein can be formulated in liposomes or other similar vesicles. Liposomes are spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review).

Vesicles can be made from several different types of lipids; however, phospholipids are most commonly used to generate liposomes as drug carriers. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

Exosomes can also be used as drug delivery vehicles for the compositions and systems described herein. For a review, see Ha et al. July 2016. Acta Pharmaceutica Sinica B. Volume 6, Issue 4, Pages 287-296; https://doi.org/10.1016/j.apsb.2016.02.001.

Ex vivo differentiated red blood cells can also be used as a carrier for an agent (e.g., a dsDNA molecule, e.g., a TDSC) described herein. See, e.g., WO2015073587; WO2017123646; WO2017123644; WO2018102740; WO2016183482; WO2015153102; WO2018151829; WO2018009838; Shi et al. 2014. Proc Natl Acad Sci USA. 111(28): 10131-10136; U.S. Pat. No. 9,644,180; Huang et al. 2017. Nature Communications 8: 423; Shi et al. 2014. Proc Natl Acad Sci USA. 111(28): 10131-10136.

Fusosome compositions, e.g., as described in WO2018208728, can also be used as carriers to deliver the dsDNA molecules described herein.

### Lipid Nanoparticles:

Lipid nanoparticles (LNPs) are carriers made of ionizable lipids. LNPs are taken up by cells via endocytosis, and their properties allow endosomal escape, which allows release of the cargo into the cytoplasm of a target cell. In addition to ionizable lipids, LNPs may contain a helper lipid to promote cell binding, cholesterol to fill the gaps between the lipids, and/or a polyethylene glycol (PEG) to reduce opsonization by serum proteins and reticuloendothelial clearance. Lipid nanoparticles, in some embodiments, comprise one or more ionic lipids, such as non-cationic lipids (e.g., neutral or anionic, or zwitterionic lipids); one or more conjugated lipids (such as PEG-conjugated lipids or lipids conjugated to polymers described in Table 5 of WO2019217941; incorporated herein by reference in its entirety); one or more sterols (e.g., cholesterol); and, optionally, one or more targeting molecules (e.g., conjugated receptors, receptor ligands, antibodies); or combinations of the foregoing.

Lipids that can be used in nanoparticle formations (e.g., lipid nanoparticles) include, for example those described in Table 4 of WO2019217941, which is incorporated by reference e.g., a lipid-containing nanoparticle can comprise one or more of the lipids in Table 4 of WO2019217941. Lipid nanoparticles can include additional elements, such as polymers, such as the polymers described in Table 5 of WO2019217941, incorporated by reference.

In some embodiments, conjugated lipids, when present, can include one or more of PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-O-(2′,3′-di(tetradecanoyloxy)propyl-1-O-(w-methoxy(polyethoxy)ethyl) butanedioate (PEG-S-DMG)), PEG dialkoxypropylcarbam, N-(carbonyl-methoxypoly ethylene glycol 2000)-1,2-distearoyl-sn-glycero-3-phosphoethanolamine sodium salt, and those described in Table 2 of WO2019051289 (incorporated by reference), and combinations of the foregoing.

In some embodiments, sterols that can be incorporated into lipid nanoparticles include one or more of cholesterol or cholesterol derivatives, such as those in WO2009/127060 or US2010/0130588, which are incorporated by reference. Additional exemplary sterols include phytosterols, including those described in Eygeris et al (2020), dx.doi.org/10.1021/acs.nanolett.Oc01386, incorporated herein by reference.

In some embodiments, the lipid particle comprises an ionizable lipid, a non-cationic lipid, a conjugated lipid that inhibits aggregation of particles, and a sterol. The amounts of these components can be varied independently and to achieve desired properties. For example, in some embodiments, the lipid nanoparticle comprises an ionizable lipid is in an amount from about 20 mol % to about 90 mol % of the total lipids (in other embodiments it may be 20-70% (mol), 30-60% (mol) or 40-50% (mol); about 50 mol % to about 90 mol % of the total lipid present in the lipid nanoparticle), a non-cationic lipid in an amount from about 5 mol % to about 30 mol % of the total lipids, a conjugated lipid in an amount from about 0.5 mol % to about 20 mol % of the total lipids, and a sterol in an amount from about 20 mol % to about 50 mol % of the total lipids. The ratio of total lipid to nucleic acid can be varied as desired. For example, the total lipid to nucleic acid (mass or weight) ratio can be from about 10:1 to about 30:1.

In some embodiments, the lipid to nucleic acid ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of lipids and nucleic acid can be adjusted to provide a desired N/P ratio, for example, N/P ratio of 3, 4, 5, 6, 7, 8, 9, 10 or higher. Generally, the lipid nanoparticle formulation's overall lipid content can range from about 5 mg/ml to about 30 mg/mL.

Some non-limiting example of lipid compounds that may be used (e.g., in combination with other lipid components) to form lipid nanoparticles for the delivery of compositions described herein, e.g., nucleic acid described herein includes,

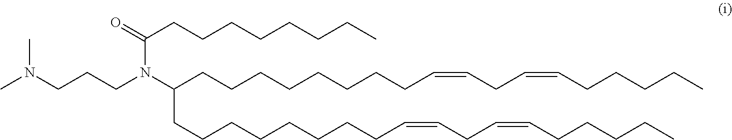 [SMILES: CCCCC/C=C\C/C=C\CCCCCCCCC(CCCCCCCC/C=C\C/C=C\CCCCC)N(CCCN(C)C)C(=O)CCCCCCCC]

In some embodiments an LNP comprising Formula (i) is used to deliver a DNA composition described herein to the liver and/or hepatocyte cells.

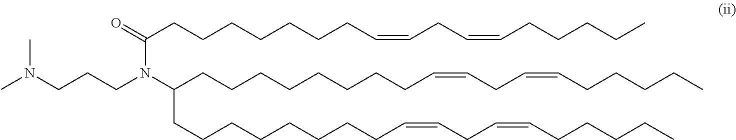 [SMILES: CCCCC/C=C\C/C=C\CCCCCCCCC(CCCCCCCC/C=C\C/C=C\CCCCC)N(CCCN(C)C)C(=O)CCCCCCC/C=C\C/C=C\CCCCC]

In some embodiments an LNP comprising Formula (ii) is used to deliver a DNA composition described herein to the liver and/or hepatocyte cells.

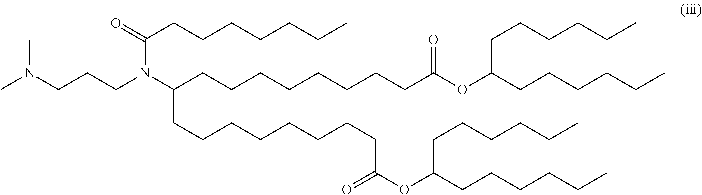 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC]

In some embodiments an LNP comprising Formula (iii) is used to deliver a DNA composition described herein to the liver and/or hepatocyte cells.

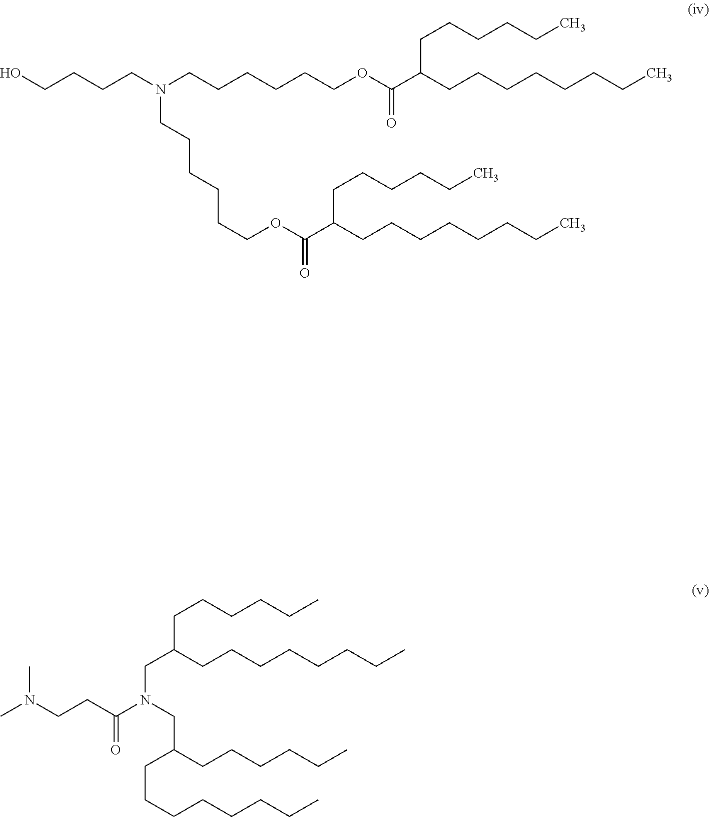

In some embodiments an LNP comprising Formula (v) is used to deliver a DNA composition described herein to the liver and/or hepatocyte cells.

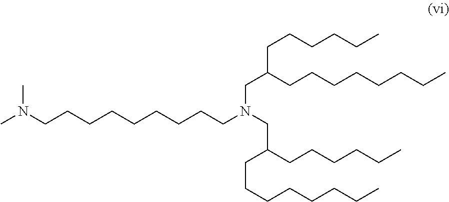 [SMILES: CCCCCCCCC(CCCCCC)CN(CCCCCCCCCN(C)C)CC(CCCCCC)CCCCCCCC]

In some embodiments an LNP comprising Formula (vi) is used to deliver a DNA composition described herein to the liver and/or hepatocyte cells.

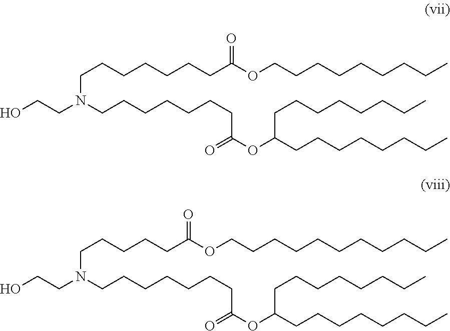 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC]

In some embodiments an LNP comprising Formula vii or (viii) is used to deliver a DNA composition described herein to the liver and/or hepatocyte cells.

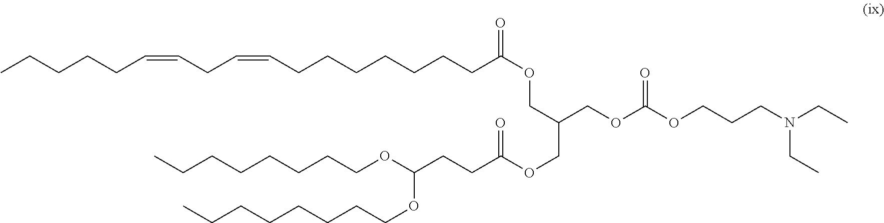 [SMILES: CCCCC/C=C\C/C=C\CCCCCCCC(=O)OCC(COC(=O)CCC(OCCCCCCCC)OCCCCCCCC)COC(=O)OCCCN(CC)CC]

In some embodiments an LNP comprising Formula (ix) is used to deliver a DNA composition described herein to the liver and/or hepatocyte cells.

In some embodiments an LNP comprising Formula (x) is used to deliver a DNA composition described herein to the liver and/or hepatocyte cells:

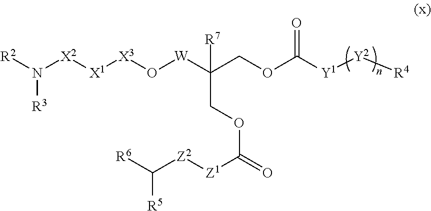

wherein
X¹ is O, NR or a direct bond X² is C2-5 alkylene, X³ is C(═O) or a direct bond, R¹ is H or Me, R³ is C1-3 alkyl, R² is C1-3 alkyl, or R² taken together with the nitrogen atom to which it is attached and 1-3 carbon atoms of X² form a 4-, 5-, or 6-membered ring, or X¹ is NR¹, R¹ and R² taken together with the nitrogen atoms to which they are attached form a 5- or 6-membered ring, or R² taken together with R³ and the nitrogen atom to which they are attached form a 5-, 6-, or 7-membered ring, Y is C2-12 alkylene, Y² is selected from

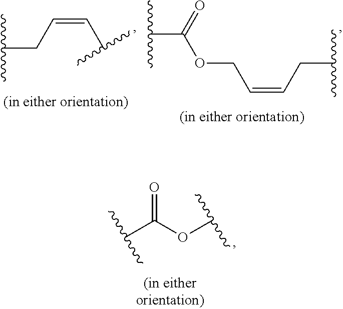 [SMILES: */C=C\C* ; *C/C=C\COC(*)=O ; *OC(*)=O]

n is 0 to 3, R⁴ is C1-15 alkyl, Z¹ is C1-6 alkylene or a direct bond,

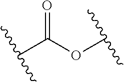 [SMILES: *OC(*)=O]

Z² is
(in either orientation) or absent, provided that if Z¹ is a direct bond, Z² is absent;
R⁵ is C5-9 alkyl or C6-10 alkoxy, R⁶ is C5-9 alkyl or C6-10 alkoxy, W is methylene or a direct bond, and R⁷ s is H or Me, or a salt thereof, provided that if R³ and R² are C2 alkyls, X¹ is O, X² is linear C3 alkylene, X³ is C(═O), Y is linear Ce alkylene, (Y²)n-R⁴ is

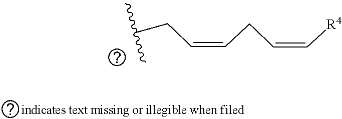

R⁴ is linear C5 alkyl T is C2 alkylene, Z² is absent, W is methylene, and R⁷ is H, then R⁵ and R⁶ are not Cx alkoxy.

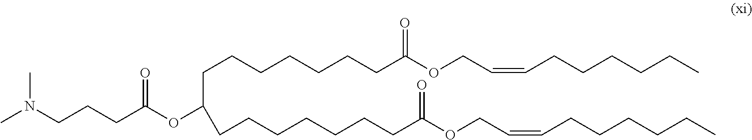 [SMILES: CCCCCC/C=C\COC(=O)CCCCCCCC(CCCCCCCC(=O)OC/C=C\CCCCCC)OC(=O)CCCN(C)C]

In some embodiments an LNP comprising Formula (xi) is used to deliver a DNA composition described herein to the liver and/or hepatocyte cells.

In some embodiments an LNP comprising Formula (xii) is used to deliver a DNA composition described herein to the liver and/or hepatocyte cells.

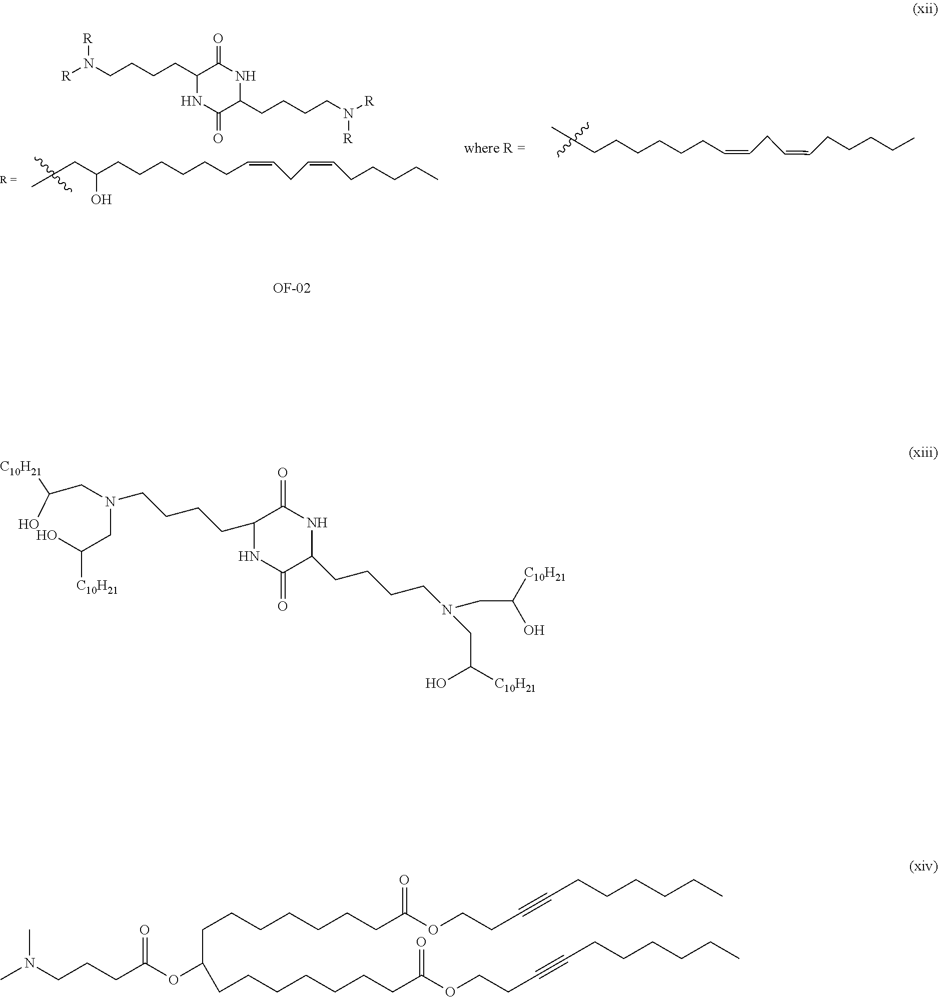

In some embodiments an LNP comprises a compound of Formula (xiii) and a compound of Formula (xiv).

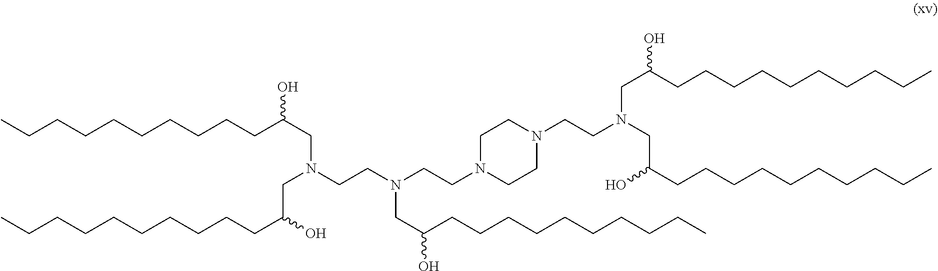 [SMILES: CCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC]

In some embodiments an LNP comprising Formula (xv) is used to deliver a DNA composition described herein to the liver and/or hepatocyte cells.

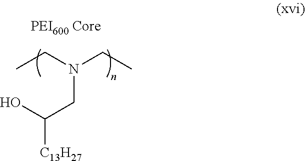

In some embodiments an LNP comprising a formulation of Formula (xvi) is used to deliver a DNA composition described herein to the lung endothelial cells.

In some embodiments an LNP comprising a formulation of Formula (xvii), (xviii), or (xix) is used to deliver a DNA composition described herein to the lung endothelial cells.

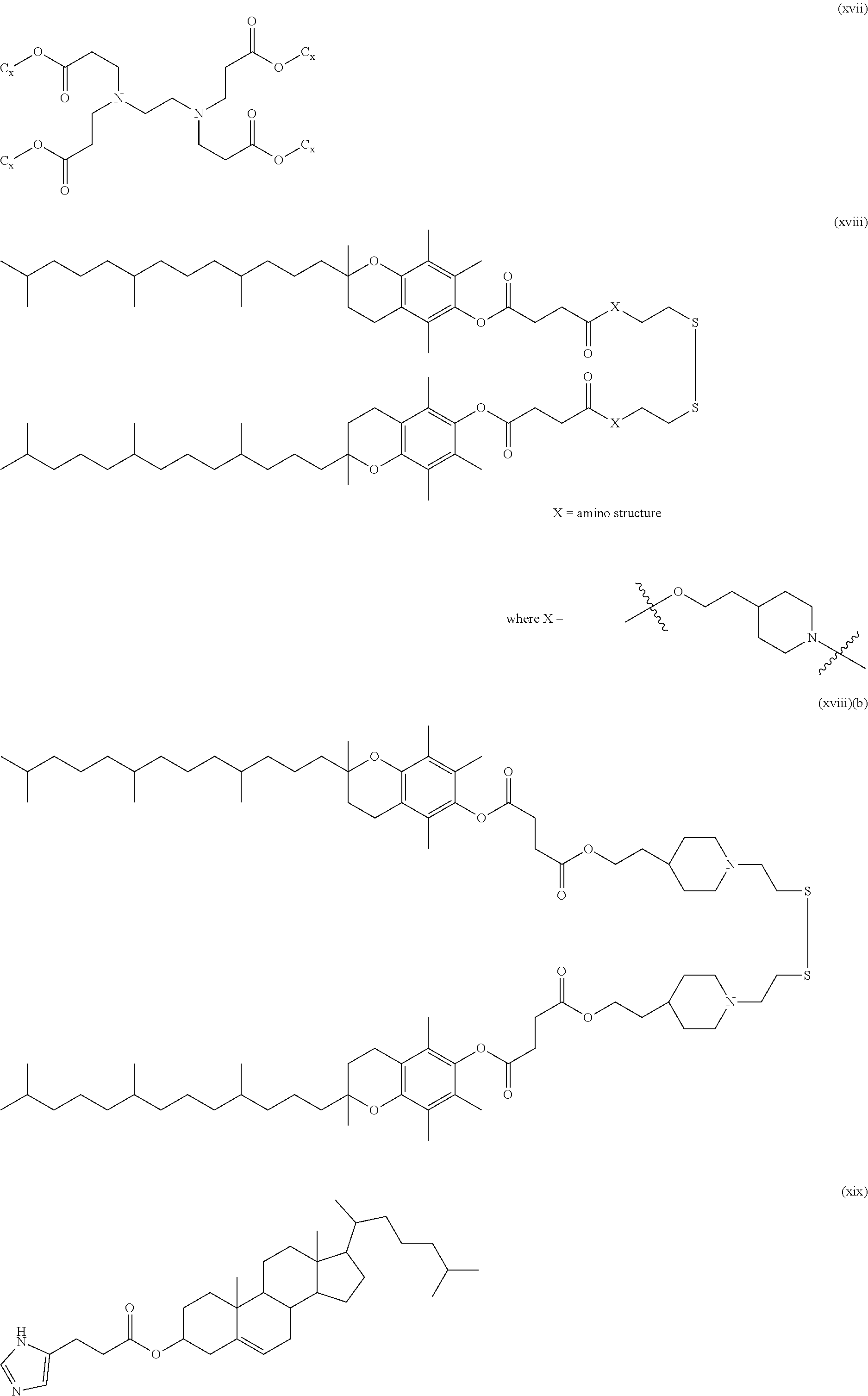

In some embodiments, a lipid compound used to form lipid nanoparticles for the delivery of compositions described herein. e.g., nucleic acid described herein is made by one of the following reactions:

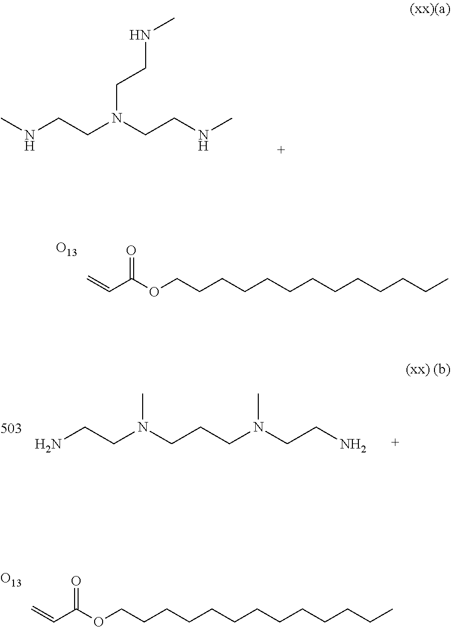

In some embodiments, a composition described herein (e.g., a nucleic acid or a protein) is provided in an LNP that comprises an ionizable lipid. In some embodiments, the ionizable lipid is heptadecan-9-yl 8-((2-hydroxyethyl)(6-oxo-6-(undecyloxy)hexyl)amino)octanoate (SM-102); e.g., as described in Example 1 of U.S. Pat. No. 9,867,888 (incorporated by reference herein in its entirety). In some embodiments, the ionizable lipid is 9Z,12Z)-3-((4,4-bis(octyloxy)butanoyl)oxy)-2-((((3-(diethylamino)propoxy)carbonyl)oxy)methyl)propyl octadeca-9,12-dienoate (LP01), e.g., as synthesized in Example 13 of WO2015/095340 (incorporated by reference herein in its entirety). In some embodiments, the ionizable lipid is Di((Z)-non-2-en-1-yl) 9-((4-dimethylamino)butanoyl)oxy)heptadecanedioate (L319), e.g. as synthesized in Example 7, 8, or 9 of US2012/0027803 (incorporated by reference herein in its entirety). In some embodiments, the ionizable lipid is 1,1′-((2-(4-(2-((2-(Bis(2-hydroxydodecyl)amino)ethyl)(2-hydroxydodecyl)amino)ethyl)piperazin-1-yl)ethyl)azanediyl)bis(dodecan-2-ol) (C12-200), e.g., as synthesized in Examples 14 and 16 of WO2010/053572 (incorporated by reference herein in its entirety). In some embodiments, the ionizable lipid is Imidazole cholesterol ester (ICE) lipid (3S,10R,13R,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 3-(1H-imidazol-4-yl)propanoate, e.g., Structure (I) from WO2020/106946 (incorporated by reference herein in its entirety).

In some embodiments, an ionizable lipid may be a cationic lipid, an ionizable cationic lipid, e.g., a cationic lipid that can exist in a positively charged or neutral form depending on pH, or an amine-containing lipid that can be readily protonated. In some embodiments, the cationic lipid is a lipid capable of being positively charged, e.g., under physiological conditions. Exemplary cationic lipids include one or more amine group(s) which bear the positive charge. In some embodiments, the lipid particle comprises a cationic lipid in formulation with one or more of neutral lipids, ionizable amine-containing lipids, biodegradable alkyne lipids, steroids, phospholipids including polyunsaturated lipids, structural lipids (e.g., sterols), PEG, cholesterol and polymer conjugated lipids. In some embodiments, the cationic lipid may be an ionizable cationic lipid. An exemplary cationic lipid as disclosed herein may have an effective pKa over 6.0. In embodiments, a lipid nanoparticle may comprise a second cationic lipid having a different effective pKa (e.g., greater than the first effective pKa), than the first cationic lipid. A lipid nanoparticle may comprise between 40 and 60 mol percent of a cationic lipid, a neutral lipid, a steroid, a polymer conjugated lipid, and a therapeutic agent, e.g., a nucleic acid described herein, encapsulated within or associated with the lipid nanoparticle. In some embodiments, the nucleic acid is co-formulated with the cationic lipid. The nucleic acid may be adsorbed to the surface of an LNP, e.g., an LNP comprising a cationic lipid. In some embodiments, the nucleic acid may be encapsulated in an LNP, e.g., an LNP comprising a cationic lipid. In some embodiments, the lipid nanoparticle may comprise a targeting moiety, e.g., coated with a targeting agent. In embodiments, the LNP formulation is biodegradable. In some embodiments, a lipid nanoparticle comprising one or more lipid described herein, e.g., Formula (i), (ii), (ii), (vii) and/or (ix) encapsulates at least 1%, at least 5%, at least 10%, at least 20%, at least 30%, at least 40%, at least 50%, at least 60%, at least 70%, at least 80%, at least 90%, at least 92%, at least 95%, at least 97%, at least 98% or 100% of a dsDNA molecule described herein.

Exemplary ionizable lipids that can be used in lipid nanoparticle formulations include, without limitation, those listed in Table 1 of WO2019051289, incorporated herein by reference. Additional exemplary lipids include, without limitation, one or more of the following formulae: X of US2016/0311759; I of US20150376115 or in US2016/0376224; I, II or III of US20160151284; I, IA, II, or IIA of US20170210967; I-c of US20150140070; A of US2013/0178541; I of US2013/0303587 or US2013/0123338; I of US2015/0141678; II, III, IV, or V of US2015/0239926; I of US2017/0119904; I or II of WO2017/117528; A of US2012/0149894; A of US2015/0057373; A of WO2013/116126; A of US2013/0090372; A of US2013/0274523; A of US2013/0274504; A of US2013/0053572; A of WO2013/016058; A of WO2012/162210; I of US2008/042973; I, II, III, or IV of US2012/01287670; I or II of US2014/0200257; I, II, or III of US2015/0203446; I or III of US2015/0005363; I, IA, IB, IC, ID, II, IIA, IIB, IIC, IID, or III-XXIV of US2014/0308304; of US2013/0338210; I, II, III, or IV of WO2009/132131; A of US2012/01011478; I or XXXV of US2012/0027796; XIV or XVII of US2012/0058144; of US2013/0323269; I of US2011/0117125; I, II, or III of US2011/0256175; I, II, III, IV, V, VI, VII, VIII, IX, X, XI, XII of US2012/0202871; I, II, III, IV, V, VI, VII, VIII, X, XII, XIII, XIV, XV, or XVI of US2011/0076335; I or II of US2006/008378; I of US2013/0123338; I or X-A-Y-Z of US2015/0064242; XVI, XVII, or XVIII of US2013/0022649; I, II, or III of US2013/0116307; I, II, or III of US2013/0116307; I or II of US2010/0062967; I-X of US2013/0189351; I of US2014/0039032; V of US2018/0028664; I of US2016/0317458; I of US2013/0195920; 5, 6, or 10 of U.S. Pat. No. 10,221,127; III-3 of WO2018/081480; I-5 or I-8 of WO2020/081938; 18 or 25 of U.S. Pat. No. 9,867,888; A of US2019/0136231; II of WO2020/219876; 1 of US2012/0027803; OF-02 of US2019/0240349; 23 of U.S. Pat. No. 10,086,013; cKK-E12/A6 of Miao et al (2020); C12-200 of WO2010/053572; 7C1 of Dahlman et al (2017); 304-013 or 503-013 of Whitehead et al; TS-P4C2 of U.S. Pat. No. 9,708,628; I of WO2020/106946; I of WO2020/106946.

In some embodiments, the ionizable lipid is MC3 (6Z,9Z,28Z,31Z)-heptatriaconta-6,9,28,31-tetraen-19-yl-4-(dimethylamino) butanoate (DLin-MC3-DMA or MC3), e.g., as described in Example 9 of WO2019051289A9 (incorporated by reference herein in its entirety). In some embodiments, the ionizable lipid is the lipid ATX-002, e.g., as described in Example 10 of WO2019051289A9 (incorporated by reference herein in its entirety). In some embodiments, the ionizable lipid is (13Z,16Z)-A,A-dimethyl-3-nonyldocosa-13,16-dien-1-amine (Compound 32), e.g., as described in Example 11 of WO2019051289A9 (incorporated by reference herein in its entirety). In some embodiments, the ionizable lipid is Compound 6 or Compound 22, e.g., as described in Example 12 of WO2019051289A9 (incorporated by reference herein in its entirety).

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid, cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C10-C24 carbon chains, e.g., lauroyl, myristoyl, palmitoyl, stearoyl, or oleoyl. Additional exemplary lipids, in certain embodiments, include, without limitation, those described in Kim et al. (2020) dx.doi.org/10.1021/acs.nanolett.Oc01386, incorporated herein by reference. Such lipids include, in some embodiments, plant lipids found to improve liver transfection with mRNA (e.g., DGTS).

Other examples of non-cationic lipids suitable for use in the lipid nanoparticles include, without limitation, nonphosphorous lipids such as, e.g., stearylamine, dodeeylamine, hexadecylamine, acetyl palmitate, glycerol ricinoleate, hexadecyl stereate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, and the like. Other non-cationic lipids are described in WO2017/099823 or US patent publication US2018/0028664, the contents of which is incorporated herein by reference in their entirety.

In some embodiments, the non-cationic lipid is oleic acid or a compound of Formula I, II, or IV of US2018/0028664, incorporated herein by reference in its entirety. The non-cationic lipid can comprise, for example, 0-30% (mol) of the total lipid present in the lipid nanoparticle. In some embodiments, the non-cationic lipid content is 5-20% (mol) or 10-15% (mol) of the total lipid present in the lipid nanoparticle. In embodiments, the molar ratio of ionizable lipid to the neutral lipid ranges from about 2:1 to about 8:1 (e.g., about 2:1, 3:1, 4:1, 5:1, 6:1, 7:1, or 8:1).

In some embodiments, the lipid nanoparticles do not comprise any phospholipids.

In some aspects, the lipid nanoparticle can further comprise a component, such as a sterol, to provide membrane integrity. One exemplary sterol that can be used in the lipid nanoparticle is cholesterol and derivatives thereof. Non-limiting examples of cholesterol derivatives include polar analogues such as 5a-choiestanol, 53-coprostanol, choiesteryl-(2′-hydroxy)-ethyl ether, choiesteryl-(4′-hydroxy)-butyl ether, and 6-ketocholestanol; non-polar analogues such as 5a-cholestane, cholestenone, 5a-cholestanone, 5p-cholestanone, and cholesteryl decanoate; and mixtures thereof. In some embodiments, the cholesterol derivative is a polar analogue, e.g., choiesteryl-(4′-hydroxy)-butyl ether. Exemplary cholesterol derivatives are described in PCT publication WO2009/127060 and US patent publication US2010/0130588, each of which is incorporated herein by reference in its entirety.

In some embodiments, the component providing membrane integrity, such as a sterol, can comprise 0-50% (mol) (e.g., 0-10%, 10-20%, 20-30%, 30-40%, or 40-50%) of the total lipid present in the lipid nanoparticle. In some embodiments, such a component is 20-50% (mol) 30-40% (mol) of the total lipid content of the lipid nanoparticle.

In some embodiments, the lipid nanoparticle can comprise a polyethylene glycol (PEG) or a conjugated lipid molecule. Generally, these are used to inhibit aggregation of lipid nanoparticles and/or provide steric stabilization. Exemplary conjugated lipids include, but are not limited to, PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. In some embodiments, the conjugated lipid molecule is a PEG-lipid conjugate, for example, a (methoxy polyethylene glycol)-conjugated lipid.

Exemplary PEG-lipid conjugates include, but are not limited to, PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-O-(2′,3′-di(tetradecanoyloxy)propyl-1-O-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, or a mixture thereof. Additional exemplary PEG-lipid conjugates are described, for example, in U.S. Pat. Nos. 5,885,613, 6,287,591, US2003/0077829, US2003/0077829, US2005/0175682, US2008/0020058, US2011/0117125, US2010/0130588, US2016/0376224, US2017/0119904, and US/099823, the contents of all of which are incorporated herein by reference in their entirety. In some embodiments, a PEG-lipid is a compound of Formula III, III-a-I, III-a-2, III-b-1, III-b-2, or V of US2018/0028664, the content of which is incorporated herein by reference in its entirety. In some embodiments, a PEG-lipid is of Formula II of US20150376115 or US2016/0376224, the content of both of which is incorporated herein by reference in its entirety. In some embodiments, the PEG-DAA conjugate can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy(polyethylene glycol)-2000]. In some embodiments, the PEG-lipid comprises PEG-DMG, 1,2-dimyristoyl-sn-glycero-3-phosphoethanolamine-N-[methoxy(polyethylene glycol)-2000]. In some embodiments, the PEG-lipid comprises a structure selected from:

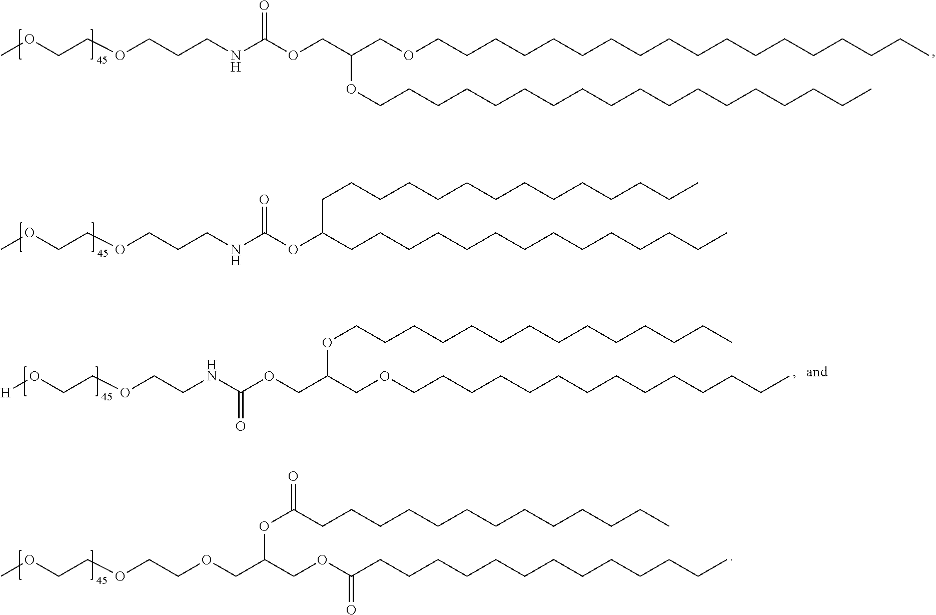

In some embodiments, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (GPL) conjugates can be used in place of or in addition to the PEG-lipid.

Exemplary conjugated lipids, i.e., PEG-lipids, (POZ)-lipid conjugates, ATTA-lipid conjugates and cationic polymer-lipids are described in the PCT and LIS patent applications listed in Table 2 of WO2019051289A9, the contents of all of which are incorporated herein by reference in their entirety.

In some embodiments, the PEG or the conjugated lipid can comprise 0-20% (mol) of the total lipid present in the lipid nanoparticle. In some embodiments, PEG or the conjugated lipid content is 0.5-10% or 2-5% (mol) of the total lipid present in the lipid nanoparticle. Molar ratios of the ionizable lipid, non-cationic-lipid, sterol, and PEG/conjugated lipid can be varied as needed. For example, the lipid particle can comprise 30-70% ionizable lipid by mole or by total weight of the composition, 0-60% cholesterol by mole or by total weight of the composition, 0-30% non-cationic-lipid by mole or by total weight of the composition and 1-10% conjugated lipid by mole or by total weight of the composition. Preferably, the composition comprises 30-40% ionizable lipid by mole or by total weight of the composition, 40-50% cholesterol by mole or by total weight of the composition, and 10-20% non-cationic-lipid by mole or by total weight of the composition. In some other embodiments, the composition is 50-75% ionizable lipid by mole or by total weight of the composition, 20-40% cholesterol by mole or by total weight of the composition, and 5 to 10% non-cationic-lipid, by mole or by total weight of the composition and 1-10% conjugated lipid by mole or by total weight of the composition. The composition may contain 60-70% ionizable lipid by mole or by total weight of the composition, 25-35% cholesterol by mole or by total weight of the composition, and 5-10% non-cationic-lipid by mole or by total weight of the composition. The composition may also contain up to 90% ionizable lipid by mole or by total weight of the composition and 2 to 15% non-cationic lipid by mole or by total weight of the composition. The formulation may also be a lipid nanoparticle formulation, for example comprising 8-30% ionizable lipid by mole or by total weight of the composition, 5-30% non-cationic lipid by mole or by total weight of the composition, and 0-20% cholesterol by mole or by total weight of the composition; 4-25% ionizable lipid by mole or by total weight of the composition, 4-25% non-cationic lipid by mole or by total weight of the composition, 2 to 25% cholesterol by mole or by total weight of the composition, 10 to 35% conjugate lipid by mole or by total weight of the composition, and 5% cholesterol by mole or by total weight of the composition; or 2-30% ionizable lipid by mole or by total weight of the composition, 2-30% non-cationic lipid by mole or by total weight of the composition, 1 to 15% cholesterol by mole or by total weight of the composition, 2 to 35% conjugate lipid by mole or by total weight of the composition, and 1-20% cholesterol by mole or by total weight of the composition; or even up to 90% ionizable lipid by mole or by total weight of the composition and 2-10% non-cationic lipids by mole or by total weight of the composition, or even 100% cationic lipid by mole or by total weight of the composition. In some embodiments, the lipid particle formulation comprises ionizable lipid, phospholipid, cholesterol and a PEG-ylated lipid in a molar ratio of 50:10:38.5:1.5. In some other embodiments, the lipid particle formulation comprises ionizable lipid, cholesterol and a PEG-ylated lipid in a molar ratio of 60:38.5:1.5.

In some embodiments, the lipid particle comprises ionizable lipid, non-cationic lipid (e.g. phospholipid), a sterol (e.g., cholesterol) and a PEG-ylated lipid, where the molar ratio of lipids ranges from 20 to 70 mole percent for the ionizable lipid, with a target of 40-60, the mole percent of non-cationic lipid ranges from 0 to 30, with a target of 0 to 15, the mole percent of sterol ranges from 20 to 70, with a target of 30 to 50, and the mole percent of PEG-ylated lipid ranges from 1 to 6, with a target of 2 to 5.

In some embodiments, the lipid particle comprises ionizable lipid/non-cationic-lipid/sterol/conjugated lipid at a molar ratio of 50:10:38.5:1.5.

In an aspect, the disclosure provides a lipid nanoparticle formulation comprising phospholipids, lecithin, phosphatidylcholine and phosphatidylethanolamine.

In some embodiments, one or more additional compounds can also be included. Those compounds can be administered separately, or the additional compounds can be included in the lipid nanoparticles of the invention. In other words, the lipid nanoparticles can contain other compounds in addition to the nucleic acid or at least a second nucleic acid, different than the first. Without limitations, other additional compounds can be selected from the group consisting of small or large organic or inorganic molecules, monosaccharides, disaccharides, trisaccharides, oligosaccharides, polysaccharides, peptides, proteins, peptide analogs and derivatives thereof, peptidomimetics, nucleic acids, nucleic acid analogs and derivatives, an extract made from biological materials, or any combinations thereof.

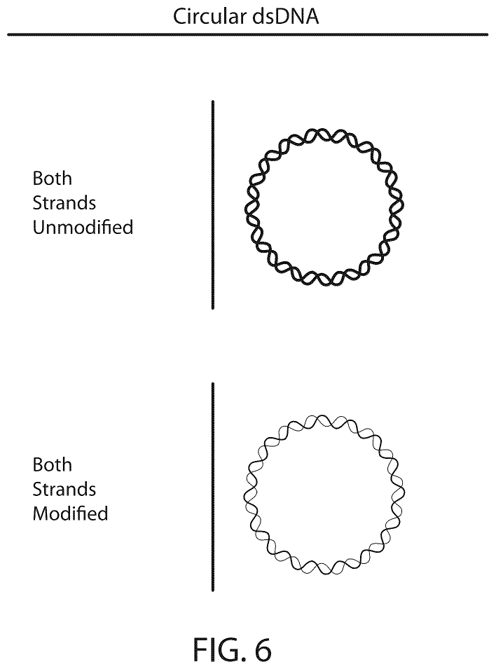

In some embodiments, LNPs are directed to specific tissues by the addition of targeting domains. For example, biological ligands may be displayed on the surface of LNPs to enhance interaction with cells displaying cognate receptors, thus driving association with and cargo delivery to tissues wherein cells express the receptor. In some embodiments, the biological ligand may be a ligand that drives delivery to the liver, e.g., LNPs that display GalNAc result in delivery of nucleic acid cargo to hepatocytes that display asialoglycoprotein receptor (ASGPR). The work of Akinc et al. Mol Ther 18(7):1357-1364 (2010) teaches the conjugation of a trivalent GalNAc ligand to a PEG-lipid (GalNAc-PEG-DSG) to yield LNPs dependent on ASGPR for observable LNP cargo effect (see, e.g., FIG. 6 of Akinc et al. 2010, supra). Other ligand-displaying LNP formulations, e.g., incorporating folate, transferrin, or antibodies, are discussed in WO2017223135, which is incorporated herein by reference in its entirety, in addition to the references used therein, namely Kolhatkar et al., Curr Drug Discov Technol. 2011 8:197-206; Musacchio and Torchilin, Front Biosci. 2011 16:1388-1412; Yu et al., Mol Membr Biol. 2010 27:286-298; Patil et al., Crit Rev Ther Drug Carrier Syst. 2008 25:1-61; Benoit et al., Biomacromolecules. 2011 12:2708-2714; Zhao et al., Expert Opin Drug Deliv. 2008 5:309-319; Akinc et al., Mol Ther. 2010 18:1357-1364; Srinivasan et al., Methods Mol Biol. 2012 820:105-116; Ben-Arie et al., Methods Mol Biol. 2012 757:497-507; Peer 2010 J Control Release. 20:63-68; Peer et al., Proc Natl Acad Sci USA. 2007 104:4095-4100; Kim et al., Methods Mol Biol. 2011 721:339-353; Subramanya et al., Mol Ther. 2010 18:2028-2037; Song et al., Nat Biotechnol. 2005 23:709-717; Peer et al., Science. 2008 319:627-630; and Peer and Lieberman, Gene Ther. 2011 18:1127-1133.

In some embodiments, LNPs are selected for tissue-specific activity by the addition of a Selective ORgan Targeting (SORT) molecule to a formulation comprising traditional components, such as ionizable cationic lipids, amphipathic phospholipids, cholesterol and poly(ethylene glycol) (PEG) lipids. The teachings of Cheng et al. Nat Nanotechnol 15(4):313-320 (2020) demonstrate that the addition of a supplemental “SORT” component precisely alters the in vivo RNA delivery profile and mediates tissue-specific (e.g., lungs, liver, spleen) gene delivery and editing as a function of the percentage and biophysical property of the SORT molecule.

In some embodiments, the LNPs comprise biodegradable, ionizable lipids. In some embodiments, the LNPs comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g., lipids of WO2019/067992, WO/2017/173054, WO2015/095340, and WO2014/136086, as well as references provided therein. In some embodiments, the term cationic and ionizable in the context of LNP lipids is interchangeable, e.g., wherein ionizable lipids are cationic depending on the pH.

In some embodiments, the average LNP diameter of the LNP formulation may be between 10s of nm and 100s of nm, e.g., measured by dynamic light scattering (DLS). In some embodiments, the average LNP diameter of the LNP formulation may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the average LNP diameter of the LNP formulation may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In some embodiments, the average LNP diameter of the LNP formulation may be from about 70 nm to about 100 nm. In a particular embodiment, the average LNP diameter of the LNP formulation may be about 80 nm. In some embodiments, the average LNP diameter of the LNP formulation may be about 100 nm. In some embodiments, the average LNP diameter of the LNP formulation ranges from about 1 mm to about 500 mm, from about 5 mm to about 200 mm, from about 10 mm to about 100 mm, from about 20 mm to about 80 mm, from about 25 mm to about 60 mm, from about 30 mm to about 55 mm, from about 35 mm to about 50 mm, or from about 38 mm to about 42 mm.

A LNP may, in some instances, be relatively homogenous. A polydispersity index may be used to indicate the homogeneity of a LNP, e.g., the particle size distribution of the lipid nanoparticles. A small (e.g., less than 0.3) polydispersity index generally indicates a narrow particle size distribution. A LNP may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a LNP may be from about 0.10 to about 0.20.

The zeta potential of a LNP may be used to indicate the electrokinetic potential of the composition. In some embodiments, the zeta potential may describe the surface charge of an LNP. Lipid nanoparticles with relatively low charges, positive or negative, are generally desirable, as more highly charged species may interact undesirably with cells, tissues, and other elements in the body. In some embodiments, the zeta potential of a LNP may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a protein and/or nucleic acid, describes the amount of protein and/or nucleic acid that is encapsulated or otherwise associated with a LNP after preparation, relative to the initial amount provided. The encapsulation efficiency is desirably high (e.g., close to 100%). The encapsulation efficiency may be measured, for example, by comparing the amount of protein or nucleic acid in a solution containing the lipid nanoparticle before and after breaking up the lipid nanoparticle with one or more organic solvents or detergents. An anion exchange resin may be used to measure the amount of free protein or nucleic acid in a solution. Fluorescence may be used to measure the amount of free protein and/or nucleic acid in a solution. For the lipid nanoparticles described herein, the encapsulation efficiency of a protein and/or nucleic acid may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In some embodiments, the encapsulation efficiency may be at least 90%. In some embodiments, the encapsulation efficiency may be at least 95%.

A LNP may optionally comprise one or more coatings. In some embodiments, a LNP may be formulated in a capsule, film, or table having a coating. A capsule, film, or tablet including a composition described herein may have any useful size, tensile strength, hardness or density.

Additional exemplary lipids, formulations, methods, and characterization of LNPs are taught by WO2020061457, which is incorporated herein by reference in its entirety. See also: Hou et al. Lipid nanoparticles for mRNA delivery. Nat Rev Mater (2021). https://doi.org/10.1038/s41578-021-00358-0.

In some embodiments, in vitro or ex vivo cell lipofections are performed using Lipofectamine MessengerMax (Thermo Fisher) or TransIT-mRNA Transfection Reagent (Mirus Bio). In certain embodiments, LNPs are formulated using the GenVoy_ILM ionizable lipid mix (Precision NanoSystems). In certain embodiments, LNPs are formulated using 2,2-dilinoleyl-4-dimethylaminoethyl-[1,3]-dioxolane (DLin-KC2-DMA) or dilinoleylmethyl-4-dimethylaminobutyrate (DLin-MC3-DMA or MC3), the formulation and in vivo use of which are taught in Jayaraman et al. Angew Chem Int Ed Engi 51(34):8529-8533 (2012), incorporated herein by reference in its entirety.

LNP formulations optimized for the delivery of CRISPR-Cas systems, e.g., Cas9-gRNA RNP, gRNA, Cas9 mRNA, are described in WO2019067992 and WO2019067910, both incorporated by reference.

Additional specific LNP formulations useful for delivery of nucleic acids are described in U.S. Pat. Nos. 8,158,601 and 8,168,775, both incorporated by reference, which include formulations used in patisiran, sold under the name ONPATTRO.

The following embodiments are contemplated:
A. A lipid nanoparticle (LNP) comprising a dsDNA molecule (e.g., a TDSC) construct, sequence or composition described herein.
B. The LNP of embodiment A, comprising a cationic lipid.
C. The LNP of embodiment B, wherein the cationic lipid has a structure according to:

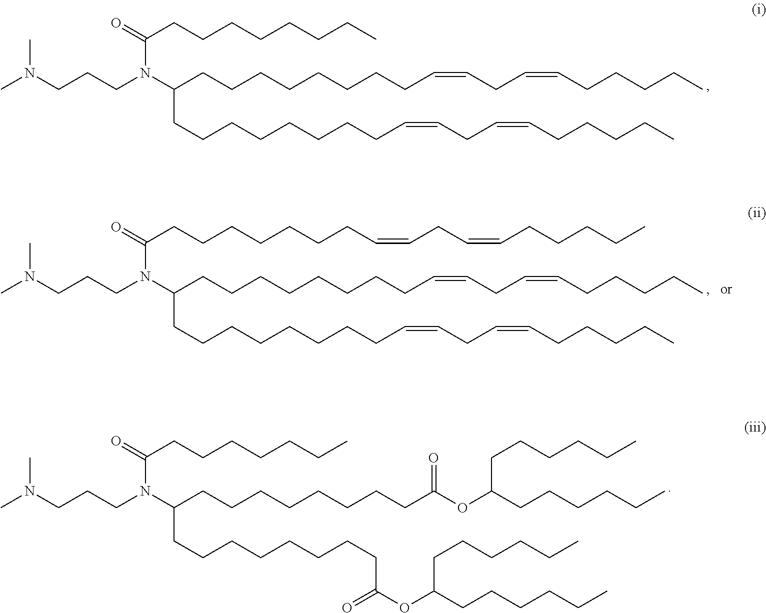

D. The LNP of any of embodiments A-C, further comprising one or more neutral lipid, e.g., DSPC, DPPC, DMPC, DOPC, POPC, DOPE, SM, a steroid, e.g., cholesterol, and/or one or more polymer conjugated lipid, e.g., a pegylated lipid, e.g., PEG-DAG, PEG-PE, PEG-S-DAG, PEG-cer or a PEG dialkyoxypropylcarbamate.

In embodiments, an LNP preparation comprising a dsDNA molecule described herein can be targeted to the desired cell type by surface decoration with targeting effectors. Such targeting effectors include, e.g., cell specific receptor ligands that bind a target cell; antibodies or other binders against a target cell; centryins; cell penetrating peptides; peptides that enable endosomal escape (e.g., GALA, KALA). See, e.g., Tables 1 and 2 of Tai & Gao. 2017. Adv Drug Deliv Rev. 110-111:157-168, for a review.

In embodiments, an LNP preparation comprising a dsDNA molecule described herein can be co-administered with an adjuvant, e.g., co-delivered in the same preparation with an adjuvant.

### Route of Administration

A dsDNA molecule (e.g., TDSC) described herein is introduced into a cell, tissue or subject by any suitable route.

Administration to a target cell or tissue (e.g., ex vivo) may be by methods known in the art such as transfection, e.g., transient or stable transfection using reagents (e.g., liposomal, calcium phosphate) or physical means (e.g., electroporation, gene gun, microinjection, microfluidic fluid shear, cell squeezing). Other methods are described, e.g., in Rad et al. 2021. Adv. Mater. 33:2005363, which is incorporated herein by reference.

Administration to a subject, e.g., a mammal, e.g., a human subject, may be by parenteral (e.g., intravenous, intramuscular, intraperitoneal, subcutaneous, or intracranial) route; by topical administration, transdermal administration or transcutaneous administration. Other suitable routes include oral, rectal, transmucosal, intranasal, inhalation (e.g., via an aerosol), buccal (e.g., sublingual), vaginal, intrathecal, intraocular, transdermal, intraendothelial, in utero (or in ovo), intrapleural, intracerebral, intraarticular, topical, intralymphatic. Also included is direct tissue or organ injection (e.g., to liver, eye, skeletal muscle, cardiac muscle, diaphragm, muscle or brain).

### Applications

The dsDNA molecule (e.g., TDSC) described herein can be used in therapeutic or health applications for a subject, e.g., a human or non-human animal. Although the descriptions of pharmaceutical compositions provided herein are principally directed to pharmaceutical compositions which are suitable for administration to humans, it will be understood by the skilled artisan that such compositions are generally suitable for administration to any other animal. The subject can be any animal, e.g., a mammal, e.g., a human or non-human mammal. In embodiments, the subject is a vertebrate animal (e.g., mammal, bird, fish, reptile, or amphibian). In embodiments, the subject is a human. In embodiments, the method subject is a non-human mammal. In embodiments, the subject is a non-human mammal is such as a non-human primate (e.g., monkeys, apes), ungulate (e.g., cattle, buffalo, sheep, goat, pig, camel, llama, alpaca, deer, horses, donkeys), carnivore (e.g., dog, cat), rodent (e.g., rat, mouse), or lagomorph (e.g., rabbit). In embodiments, the subject is a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots). In embodiments, the subject is an invertebrate such as an arthropod (e.g., insects, arachnids, crustaceans), a nematode, an annelid, a helminth, or a mollusk.

In some embodiments, a DNA described herein is provided at a dose of about 0.1-100 mg/kg of the DNA.

In some embodiments, a dsDNA molecule described herein imparts a biological effect of the effector, e.g., expression of a therapeutic polypeptide, on a host cell, tissue or subject over a time period of at least 2, at least 3, at least 4, at least 5, at least 6 days or at least a week; at least 8, at least 9, at least 10, at least 12, at least 14 days or at least two weeks; at least 16, at least 18, at least 20 days or at least 3 weeks; at least 22, at least 24, at least 25, at least 27, at least 28 days or at least a month; at least 2 months, at least 3 months, at least 4 months, at least 5 months, at least 6 months or more; between one week and 6 months, between 1 month to 6 months, or between 3 months to 6 months.

In some embodiments, a dsDNA molecule described herein imparts a biological effect of the effector, e.g., expression of a therapeutic polypeptide, on a host cell, tissue or subject over a time period of at least 1 cell divisions of the host cell.

In embodiments, a dsDNA molecule described herein can be used to deliver an effector, e.g., an effector described herein, to a cell, tissue or subject.

In embodiments, a dsDNA molecule described herein can be used to modulate (e.g., increase or decrease) a biological parameter in a cell, tissue or subject. The biological parameter may be an increase or decrease in gene expression of a subject gene in a target cell, tissue or subject.

In embodiments, a dsDNA molecule described herein can be used to treat a cell, tissue or subject in need thereof by administering a dsDNA molecule described herein to such cell, tissue or subject.

In embodiments, the dsDNA molecule delivers an effector to a cell chosen from an immune cell (e.g., a monocyte), a cancer cell, a HEK293 cell, a hepatocyte, or an epidermal cell (e.g., a keratinocyte).

### EXAMPLES

### Table of Contents

Example 1: Formulation of a dsDNA molecule (e.g., TDSC) with LNP
Example 2: Determining exonuclease resistance for a dsDNA molecule (e.g., TDSC) comprising closed ends
Example 3: Determining exonuclease resistance for a dsDNA molecule (e.g., TDSC) comprising an open end (e.g., two open ends)
Example 4: Design and assembly of a plasmid template for production of double-stranded DNA (dsDNA) molecules
Example 5: Production of dsDNA molecules with chemical modifications
Example 6: Assessment of reporter gene expression in vitro
Example 7: Assessment of innate immune response in cells in vitro
Example 8: Quantification of DNA chemical modifications in vitro
Example 9: Validation of chemically modified DNA sequences in cells
Example 10: Assessment of reporter gene expression in vitro
Example 11: Assessment of innate immune response in cells in vitro
Example 12: Assessment of mutations during production of dsDNA molecules

### Example 1: Formulation of a dsDNA Molecule (e.g., TDSC) with LNP

This example describes how to formulate the constructs made as described herein with a lipid nanoparticle (LNP).

Nucleic acid constructs are combined with lipid components via microfluidic devices according to the method of Chen et al. 2012. J Am Chem Soc. Volume 134, Issue 16:6948-6951. Briefly, the microfluidic devices are fabricated in polydimethylsiloxane (PDMS) according to standard lithographic procedures (McDonald & Whitesides. 2002. Accounts Chem Res Volume 35, Issue 7:491-499). The lipid components, typically containing cationic lipids, cholesterol, helper lipids, polyethylene glycol modified lipids, and lipids facilitating targeting moiety conjugation (optional), are combined and solubilized in 90% ethanol. The nucleic acid constructs are dissolved in buffer. The nucleic acid solution, the lipid solution, and phosphate buffer saline (PBS) are injected into the microfluidic device. The freshly prepared LNPs are dialyzed against PBS buffer using membranes with MWCO of 3.5 kD to remove ethanol and exchange buffer.

The LNPs are characterized in terms of effective diameter, polydispersity, and zeta potential using dynamic light scattering (DLS) (ZetaPALS, Brookhaven Instruments, NY, 15-mW laser, incident beam 676 nm); and total nucleic acid concentration is determined by lysing the particles and using Quant-iT™ 1×dsDNA Assay Kits, High Sensitivity (HS) and Broad Range (BR) according to the manufacturer protocols (ThermoFisher Scientific, Q33232).

### Example 2: Determining Exonuclease Resistance for a dsDNA Molecule (e.g., TDSC) Comprising Closed Ends

This example describes how to test if a dsDNA molecule (e.g., TDSC) comprising closed ends (e.g., an adaptor-ligated linear dsDNA construct) is Exonuclease III (M0206, New England Biolabs Inc.) resistant. The TDSC is tested next to a non-nuclease control. The non-nuclease control contains DNA with the identical sequence to the TDSC of interest except that it underwent the adaptor ligation protocol that is used to add the exonuclease-resistant DNA end form to the TDSC, but without an adaptor oligonucleotide added to the mixture. 1 μL of Exonuclease III (at a starting concentration of 100 units/uL) is added per 5 μg of DNA in 50 μL. The tubes are mixed well and spun down. The tubes are run on the thermocycler for 1 hour at 37° C., and heat inactivated at 70° C. for 30 minutes.

The samples are purified via the Nucleospin® Gel and PCR Clean-up kit (catalog #740609, Macherey-Nagel) using a vacuum manifold according to manufacturer protocols. Briefly, the elution buffer is warmed to 70° C. 2× volumes of NTI binding buffer are added to 1× volume of Exo Ill-treated DNA. The samples are mixed until evenly distributed and left at room temperature for 5 minutes. The column on the vacuum manifold is secured, valve opened, and vacuum turned on. 375 μL DNA-NTI mix is added to 2× columns and allowed to fully pass through each column. 700 μL of NTC wash buffer is added twice. The column is removed from the vacuum manifold and placed into a collection tube. The assembly is centrifuged at 11,000×g for 1 minute. The column is placed into a new low bind microcentrifuge tube, 25 μL of prewarmed buffer is added, and the assembly is incubated at 70° C. for 5 min. The assembly is centrifuged at 11,000×g for 1 min. The incubation and elution steps are repeated a second time. The collected DNA is quantified by dsDNA BR Qubit (Q32850, Thermo Fisher Scientific) on the Qubit 4 Fluorometer (Q33226, Thermo Fisher Scientific) according to manufacturer protocols.

The samples are loaded into E-Gel EX, 1% Agarose Gel (G402021, Thermo Fisher Scientific) in individual wells at an amount of 16 ng of DNA per well. The ladder (10488090, Thermo Fisher Scientific) is loaded at 2 μl into the left most lane of the gel. The gel is run through the E-Gel Power Snap Electrophoresis System according to manufacturer protocols (G8100, G8200, Thermo Fisher Scientific). After the gel is run, the exonuclease-resistant TDSC is visible at the molecular weight corresponding to the full-length DNA plus closed-adapter sequence. A TDSC will be considered exonuclease-resistant in this assay if at least 95% of the product that appears in the gel in that lane corresponds to the full-length TDSC.

### Example 3: Determining Exonuclease Resistance for a dsDNA Molecule (e.g., TDSC) Comprising an Open End (e.g., Two Open Ends)

This example describes how to test if a dsDNA molecule (e.g., TDSC) comprising an open end (e.g., an adaptor-ligated linear dsDNA construct) is Exonuclease III (M0206, New England Biolabs Inc.) resistant. The TDSC is tested next to a non-nuclease control. The non-nuclease control contains DNA with the identical sequence to the TDSC of interest except that it underwent the adaptor ligation protocol that is used to add the exonuclease-resistant DNA end form to the TDSC, but without an adaptor oligonucleotide added to the mixture. 2 units of Exonuclease III are added per 200 ng of DNA (at 10 ng/ul), in a 20 ul reaction. The tubes are mixed well and spun down. The tubes are run on the thermocycler for 30 min at 37° C.

The samples are loaded into E-Gel EX, 1% Agarose Gel (G402021, Thermo Fisher Scientific) in individual wells at an amount of 20 ng of DNA per well. The ladder (10488090, Thermo Fisher Scientific) is loaded at 2 μl into the left most lane of the gel. The gel is run through the E-Gel Power Snap Electrophoresis System according to manufacturer protocols (G8100, G8200, Thermo Fisher Scientific). After the gel is run, the exonuclease-resistant TDSC is visible at the molecular weight corresponding to the full-length DNA plus closed-adapter sequence. A TDSC will be considered exonuclease-resistant in this assay if at least 95% of the product that appears in the gel in that lane corresponds to the full-length TDSC.

### Example 4: Design and Assembly of a Plasmid Template for Production of Double-Stranded DNA (dsDNA) Molecules

This example describes production of a plasmid template for a dsDNA molecule. In this example, a construct template was designed with the following specific sequence components.

Promoter Ef1a:
(SEQ ID NO: 37)
5′ggctccggtgcccgtcagtgggcagagcgcacatcgcccacagtcccc
gagaagttggggggaggggtcggcaattgaaccggtgcctagagaaggtg
gcgcggggtaaactgggaaagtgatgtcgtgtactggctccgcctttttc
ccgaggggggggagaaccgtatataagtgcagtagtcgccgtgaacgttc
tttttcgcaacgggtttgccgccagaacacaggtaagtgccgtgtgtggt
tcccgcgggcctggcctctttacgggttatggcccttgcgtgccttgaat
tacttccacctggctgcagtacgtgattcttgatcccgagcttcgggttg
gaagtggggggagagttcgaggccttgcgcttaaggagccccttcgcctc
gtgcttgagttgaggcctggcctgggcgctggggccgccgcgtgcgaatc
tggtggcaccttcgcgcctgtctcgctgctttcgataagtctctagccat
ttaaaatttttgatgacctgctgcgacgctttttttctggcaagatagtc
ttgtaaatgcgggccaagatctgcacactggtatttcggtttttggggcc
gcgggcggcgacggggcccgtgcgtcccagcgcacatgttcggcgagggg
ggcctgcgagcgcggccaccgagaatcggacgggggtagtctcaagctgg
ccggcctgctctggtgcctggcctcgcgccgccgtgtatcgccccgccct
gggcggcaaggctggcccggtcggcaccagttgcgtgagcggaaagatgg
ccgcttcccggccctgctgcagggagctcaaaatggaggacgcggcgctc
gggagagcggggggtgagtcacccacacaaaggaaaagggcctttccgtc
ctcagccgtcgcttcatgtgactccacggagtaccggggccgtccaggca
cctcgattagttctcgagcttttggagtacgtcgtctttaggttgggggg
aggggttttatgcgatggagtttccccacactgagtgggtggagactgaa
gttaggccagcttggcacttgatgtaattctccttggaatttgccctttt
tgagtttggatcttggttcattctcaagcctcagacagtggttcaaagtt
tttttcttccatttcaggtgtcgtga-3′
Effector sequence encoding a
model/marker protein (mCherry):
(SEQ ID NO: 38)
5′atggtgagcaagggcgaggaggataacatggccatcatcaaggagttc
atgcgcttcaaggtgcacatggagggctccgtgaacggccacgagttcga
gatcgagggcgagggcgagggccgcccctacgagggcacccagaccgcca
agctgaaggtgaccaagggtggccccctgcccttcgcctgggacatcctg
tcccctcagttcatgtacggctccaaggcctacgtgaagcaccccgccga
catccccgactacttgaagctgtccttccccgagggcttcaagtgggagc
gcgtgatgaacttcgaggacggcggcgtggtgaccgtgacccaggactcc
tccctgcaggacggcgagttcatctacaaggtgaagctgcgcggcaccaa
cttcccctccgacggccccgtaatgcagaagaagaccatgggctgggagg
cctcctccgagcggatgtaccccgaggacggcgccctgaagggcgagatc
aagcagaggctgaagctgaaggacggcggccactacgacgctgaggtcaa
gaccacctacaaggccaagaagcccgtgcagctgcccggcgcctacaacg
tcaacatcaagttggacatcacctcccacaacgaggactacaccatcgtg
gaacagtacgaacgcgccgagggccgccactccaccggcggcatggacga
gctgtacaagtaa-3′
Optional:
NTS: SV40 enhancer:
(SEQ ID NO: 1)
5′-cccaagaagaagaggaaagtc-3′
Maintenance sequence: human
interferon-β MAR
(SEQ ID NO: 39)
5′tataattcactggaatttttttgtgtgtatggtatgacatatgggttc
ccttttattttttacatataaatatatttccctgtttttctaaaaaagaa
aaagatcatcattttcccattgtaaaatgccatatttttttcataggtca
cttacata3′
Second strand motif: AAV2
wildtype ITR
(SEQ ID NO: 26)
5′aggaacccctagtgatggagttggccactccctctctgcgcgctcgct
cgctcactgaggccgggcgaccaaaggtcgcccgacgcccgggctttgcc
cgggcggcctcagtgagcgagcgagcgcgcagctgcctgcagg-3′

A plasmid template was designed with these elements using standard DNA design manipulation software. Once designed, plasmids were ordered from a commercial supplier (GenScript) for use as a template in PCR amplification.

### Example 5: Production of dsDNA Molecules with Chemical Modifications

This Example demonstrates preparation of dsDNA molecules containing cytosines with chemical modifications at the carbon 5 position (C-5 position), such as 5-formyl-2′-deoxycytosine (5-formylcytosine).

Plasmid DNA (10 ng/50 ul PCR reaction) was used as a template for PCR amplification using KOD polymerase (710864, Sigma Aldrich), KOD Xtreme (KODX) polymerase (719753, Sigma Aldrich) or Deep Vent polymerase (M0258, NEB). Other commercially available polymerases may also be used. The product versions used were purchased as constitutive components, rather than in a mastermix format, to ensure precise ratios of modified nucleotides to standard dNTPs. PCR reaction conditions for each enzyme included:
a. For the KOD polymerase, MgSO₄ at a final concentration of 2 mM.
b. 100 mM unmodified dNTP solution set (N0446, New England Biolabs), at a final concentration of 200 μM.
c. Modified deoxynucleoside triphosphates (e.g. 5-formyl-dCTP, N-2064, Trilink Biotechnologies) were added at various ratios with their cognate dNTP, summing to a total of 200 μM (i.e., 200 μM dATP, 200 μM dCTP, 200 μM dTTP, and 200 μM dGTP). Thus, a reaction designed for 25% incorporation would be 50 μM modified dCTP and 150 μM unmodified dCTP for a total of 200 μM dCTP.
d. Forward and reverse primers at a final concentration of 300 M. Thermocycling was performed according to manufacturers' protocols, except that the extension time was lengthened from 20-60 seconds per kilobase of amplicon (for KOD and KOD Xtreme polymerases, respectively) to 2-3 minutes per kilobase.

For the synthesis of linear, covalently closed dsDNA molecules, primers contained either a phosphate group for improved ligation efficiency or a TelN recognition sequence.

For the synthesis of circular dsDNA forms, in addition to containing sequences complementary to the plasmid, primers contained additional sequences useful in downstream processes:
a. Nicking enzyme(s) recognition sequence;
b. Restriction enzyme recognition sequence (e.g. BsaI, KpnI, or NheI), used to create sticky-ends in the DNA after restriction enzyme digestion and facilitate DNA circularization; and
c. Additional bases (e.g., 5′-CCGTGGTCCTTC-3′) (SEQ ID NO: 40) to increase restriction enzyme digestion efficiency.

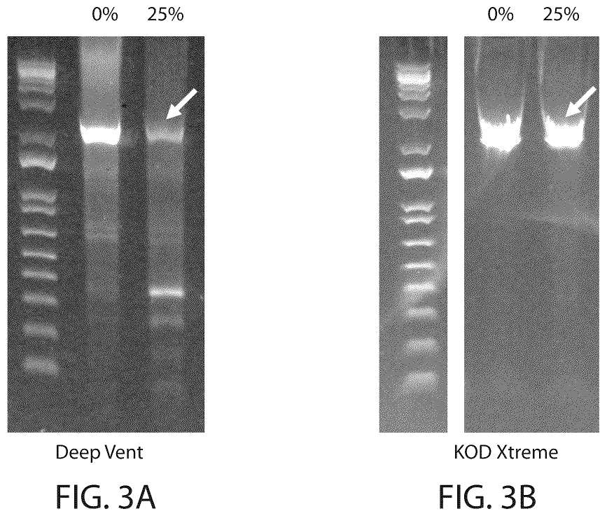

FIG. 3A and FIG. 3B show the successful incorporation of 5-formyl-dCTP into a PCR product. The desired product (indicated with an arrow) was detected when 5-formyl-dCTP was added to the PCR mix at a 1:3 ratio to unmodified dCTP (i.e., 25% 5-formyl-dCTP of total dCTP) and when either Deep Vent polymerase (FIG. 3A) or KOD Xtreme polymerase (FIG. 3B) were used.

For all forms, the PCR product was purified using standard DNA purification columns.

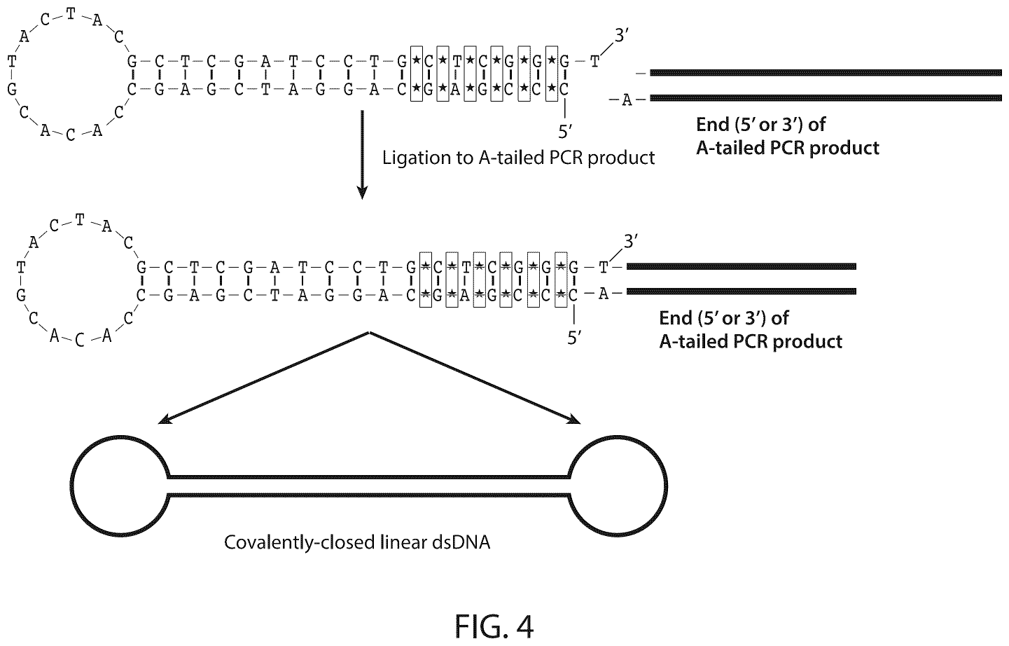

FIG. 4 depicts production of covalently closed linear dsDNA molecules with end forms comprising phosphorothioate modifications. For dsDNA molecules with end forms comprising phosphorothioate modifications, up to 10 μg in 50 μL of PCR DNA per reaction was added to the NEBNext Ultra II End Repair/dA-Tailing buffer (8 μL) and enzyme (3 μL) mixes (E7546L) and incubated first at 20° C. for 30 min, then at 65° C. for 30 min. After briefly cooling on ice, NEBNext Ultra II ligation module components were added, including ligation mix (30 μL), ligation enhancer (1 μL), and 3 μL of a 100 μM solution containing the phosphorothioated DNA adapter to be ligated. This reaction was incubated >1 hr (typically overnight). The ligated PCR-adapter solution was then purified by Nucleospin Midi columns, quantified by Nanodrop, and any non-ligated PCR was removed with Exonuclease III (NEB M0206) for one hour at 37° C.

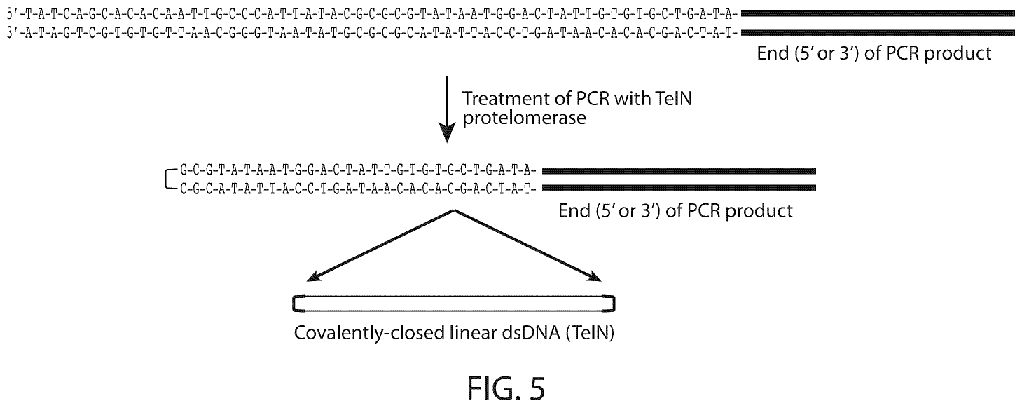

FIG. 5 depicts production of covalently closed linear dsDNA molecules with TelN end forms. For dsDNA molecules with TelN end forms, 1 μg of the purified PCR product was incubated in a 40 μL reaction containing 4 μL 10× ThermoPol buffer, 2 μL TelN protelomerase (M0651, New England Biolabs) at 30° C. for 1 hr. The TelN-modified DNA was then purified by Zymo DCC-100 columns, quantified by Nanodrop, and any PCR products without TelN modifications were removed with Exonuclease III (NEB M0206) for one hour at 37° C.

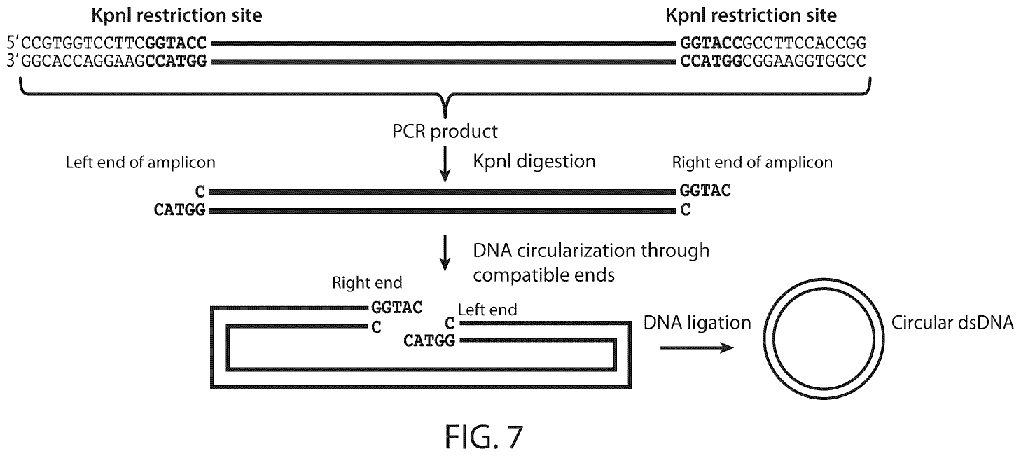

FIG. 6 is a diagram depicting circular dsDNA molecules, and FIG. 7 depicts production of circular dsDNA molecules. For the synthesis of circular dsDNA molecules, purified PCR product was digested, in an overnight reaction, using the restriction enzyme corresponding to the restriction enzyme recognition sequence, for instance, KpnI-HF-V2 (R3142, New England Biolabs). DNA was then purified using DNA purification columns. Digested DNA was circularized using T3 DNA ligase (M0317, New England Biolabs) for one hour at 26° C. Non-circularized DNA was degraded by incubating the DNA with T5 exonuclease (M0663L, New England Biolabs) for one hour at 37° C. T5 exonuclease was used to digest linear dsDNA but not circular dsDNA. DNA was purified using DNA purification columns. Other similar methods may also be used, for instance agarose gel purification.

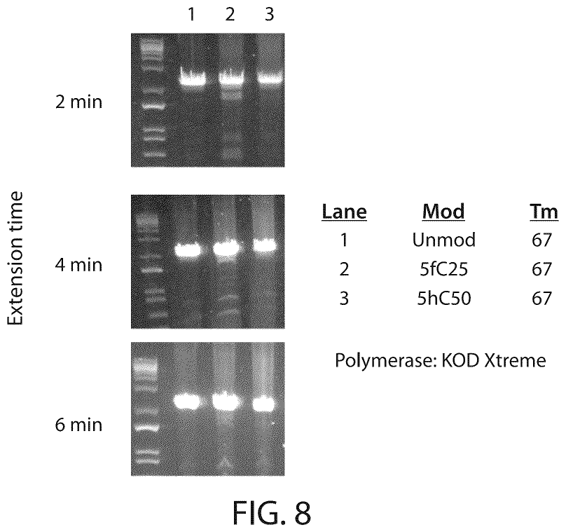

FIG. 8 shows that PCR with various thermocyling parameters reaction can be used to produce DNA molecules with chemical modifications, including 5-formylcytosine and 5-hydroxycytosine. For KOD Xtreme polymerase, extension times of two minutes (the manufacturer's recommendation), four minutes, and six minutes produced detectable amounts of chemically modified DNA amplicons, including 5-formylcytosine (lane 2) and 5-hydroxycytosine (lane 3).

Analysis of the composition and purity of the resultant dsDNA molecules was performed on an Agilent 5300 Fragment Analyzer using the CRISPR Discovery Kit (DNF-930-K1000CP). The dsDNA inlet buffer and running gel with intercalating dye was prepared fresh each day, while the marker tray with mineral oil overlay and capillary conditioning solution were prepared fresh each month. The buffers were prepared to the manufacturer's specifications. Circular dsDNA molecules were diluted in water to a final concentration of 100 pg/uL. For each sample well, 2 uL of the DNA samples were added to 22 uL of Dilution Buffer (0.1×TE), and each sample was run with 2-4 replicates, with one well used for the MDK DNA ladder. The samples were run via the instrument controller software using default settings of the CRISPR Discovery Method (CRP-910-33).

Sample traces were analyzed using the ProSize Data Analysis Software v4.0.2.7. Peak Analysis conditions for dsDNA were set at the standard conditions of a ‘Peak Width (sec)’ of 5 and a ‘Min. peak height (RFU)’ of 50, #Extra Valley Points of 3, and with ‘Valley to Valley Baseline?’ turned on. Manual baseline was set at −2 min from the lower marker and +2 min to from the upper marker. Peaks were automatically detected by the software under these conditions, and peaks widths were chosen by the software except for instances where manual adjustments were required to due to broad peaks, peak shoulders, or to multiple peaks within a narrow size range.

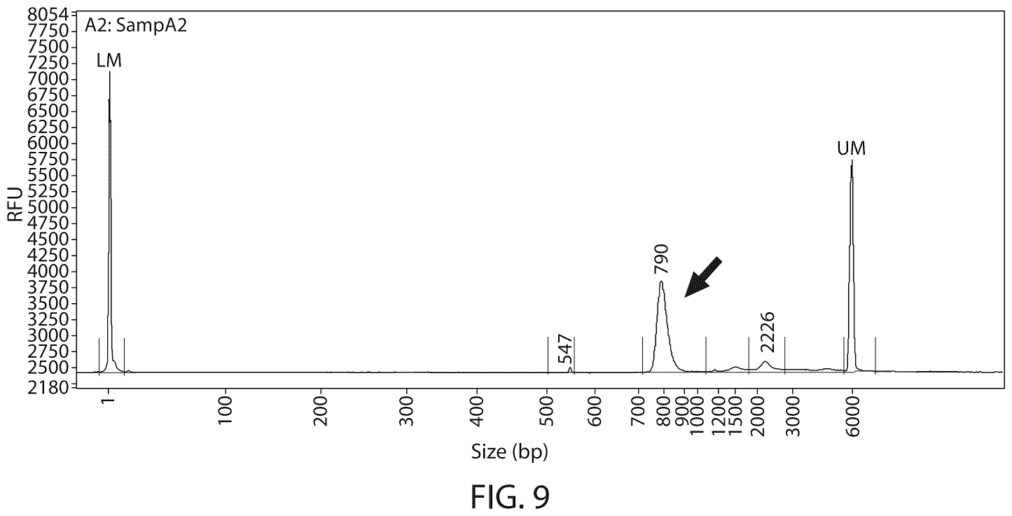

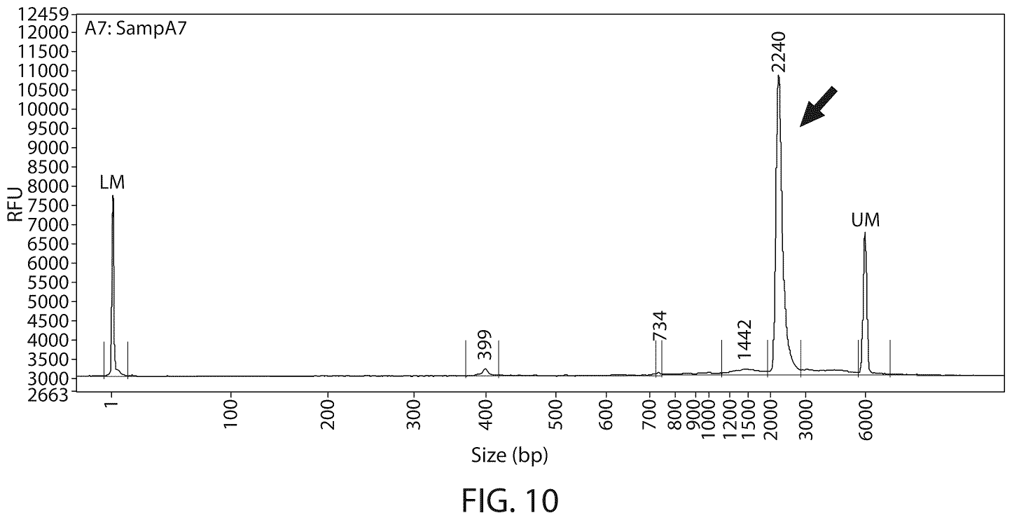

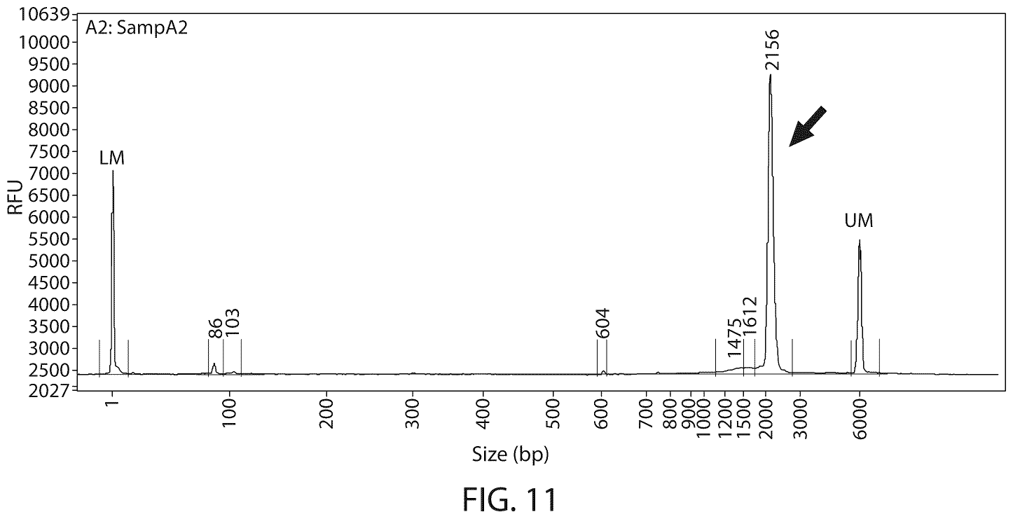

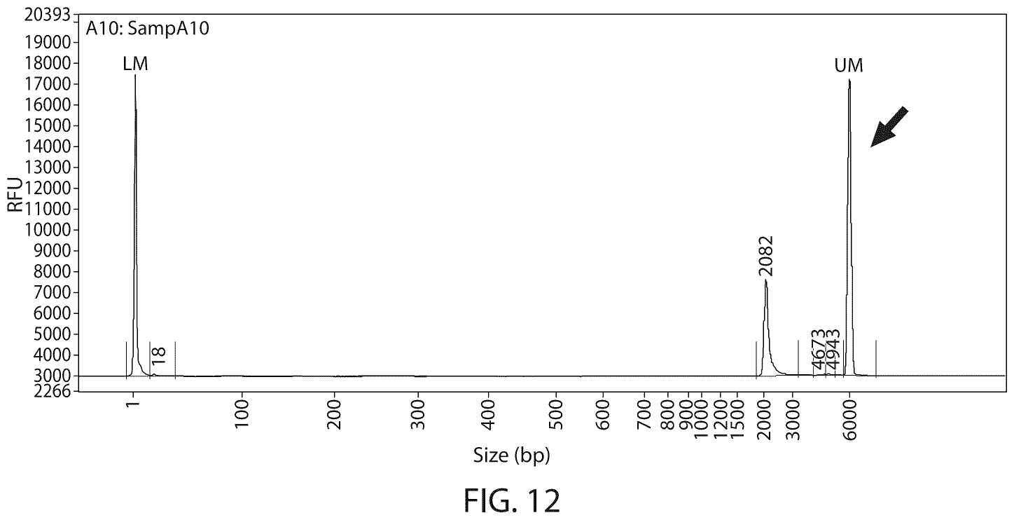

FIGS. 9-12 show fragment analyzer traces of dsDNA molecules with C-5 cytosine modifications. FIG. 9 shows a circular dsDNA molecule, produced in a reaction using 25% 5-formylcytosine and purified as described above. FIG. 10 and FIG. 11 show linear covalently closed dsDNA molecules with end forms comprising phosphorothioate modifications (FIG. 10) and TelN end forms (FIG. 11), produced in a reaction using 25% formylcytosine and purified as described above. FIG. 12 shows a linear covalently closed dsDNA molecules produced in a reaction using 50% 5-hydroxycytosine with end forms comprising phosphorothioate modifications. In each trace, a single peak (indicated with an arrow) is clearly visible. These results indicate that cytosines with 5-C chemical modifications (e.g., 5-formylcytosine and 5-hydroxycytosine) can be incorporated into multiple types of dsDNA molecules and purified.

### Example 6: Assessment of Reporter Gene Expression In Vitro

This example demonstrates detection and quantification of gene expression using chemically modified dsDNA molecules in cultured cells.

Experimental dsDNA molecules and controls were administered via lipid transfection (lipofection). Lipofection for DNA was performed using the Lipofectamine3000 transfection reagent (#L3000001, ThermoFisher) in HEKa, HepG2, HEK293, and U937 cells according to manufacturer's instructions. A 1:2:3 ratio of DNA:P3000:Lipofectamine3000 was used for all DNA constructs and controls. 10,000 cells were pre-seeded into each well of 96-well plates one day before transfection. Transfection was performed when cells reached roughly 80 to 90% confluence. For each well of a 96-well plate, 3× Lipofectamine3000 was first diluted in 5 uL of Opti-MEM™ I Reduced Serum Medium (#31985070, ThermoFisher). DNA was diluted in 5 uL Opti-MEM™ I Reduced Serum Medium with 2×P3000 reagent. The DNA was then added into the Lipofectamine3000 containing Opti-MEM™ I Reduced Serum Medium and mixed gently by pipetting. After incubating for 15 minutes at room temperature, the DNA-Lipofectamine3000 complex was added to target cells with full culture medium in a dropwise manner to different areas of the well. The plate was gently rocked back-and-forth and side-to-side to evenly distribute the DNA-Lipofectamine3000 complex. Following transfection, cells were incubated in a CO₂ tissue culture incubator, and culture medium was changed 6 to 8 hours after transfection.

To determine expression of constructs encoding the fluorescent reporter mCherry, cells were first washed with PBS before flow cytometric analysis. All flow cytometry was performed on MACSQuant VYB by Miltenyi. For detection of mCherry signal, a yellow laser (wavelength 561 nm) was used for excitation and a 615/620 nm emission filter was used. 20,000 events were recorded for each sample and data were analyzed using Flowjo V.9.0 software. Cells were first gated on FSC-A and SSC-A plot to remove cell debris. The population was further plotted on an FSC-A and FSC-H plot to circumscribe the single cell population. Finally, a bivariate plot between the fluorescent signal expressing and non-expressing cells was used to determine the percentage of expressing cells. A distribution of expressing cells was used to determine the level of expression within each cell. Expression analysis was performed at multiple time points.

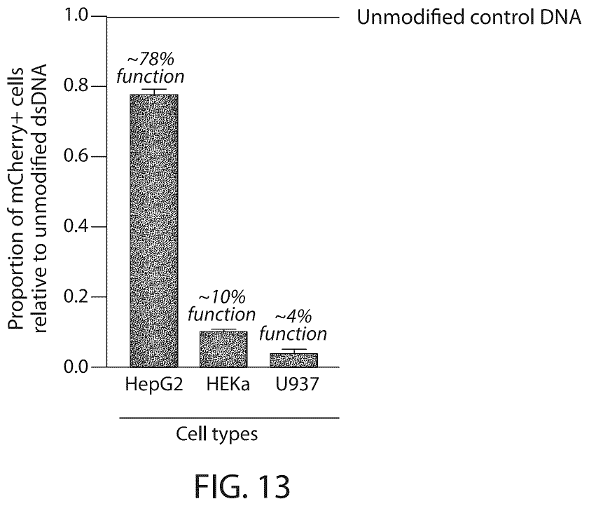

FIG. 13 shows that dsDNA chemically modified with 5-formylcytosine supported expression of a reporter protein. In three separate cell types (HepG2, HEKa, and U937 cells), 5-formylcytosine-modified dsDNA in circular form retained function, as defined by detectable expression of the reporter protein mCherry. In HepG2 cells, 5-formylcytosine-modified dsDNA was about 80% as functional as unmodified control dsDNA, as defined by the relative percentage of mCherry+ cells relative to unmodified control dsDNA. These results demonstrate that dsDNA chemically modified with 5-formylcytosine can be transcribed, and ultimately yield a protein product, in multiple cell lines.

### Example 7: Assessment of Innate Immune Response in Cells In Vitro

This example demonstrates the effect of chemically modified dsDNA molecules on the innate immune response of cultured cells.

Experimental constructs were prepared as in Examples 4 and 5 above, then administered to cells as in Example 6 above. qPCR was performed to determine the RNA level of cytokines IFN-b, IL-6, TNF-a, and CXCL10 in the cells lipofected with the dsDNA molecules. Briefly, the probe-primer sets used in qPCR were human IFN-b (forward sequence: CTTGGATTCCTACAAAGAAGCAGC (SEQ ID NO: 41); reverse sequence: TCCTCCTTCTGGAACTGCTGCA) (SEQ ID NO: 42); human IL-6 (forward sequence: AGACAGCCACTCACCTCTTCAG (SEQ ID NO: 43); reverse sequence: TTCTGCCAGTGCCTCTTTGCTG (SEQ ID NO: 44)); human TNF-a (forward sequence: CTCTTCTGCCTGCTGCACTTTG (SEQ ID NO: 47); reverse sequence: ATGGGCTACAGGCTTGTCACTC (SEQ ID NO: 48)); human CXCL10 (forward sequence: GGTGAGAAGAGATGTCTGAATCC (SEQ ID NO: 49); reverse sequence: GTCCATCCTTGGAAGCACTGCA (SEQ ID NO: 50)); human CCL20 (forward sequence: AAGTTGTCTGTGTGCGCAAATCC (SEQ ID NO: 51); reverse sequence: CCATTCCAGAAAAGCCACAGTTTT (SEQ ID NO: 52)), human GAPDH (forward sequence: GTCTCCTCTGACTTCAACAGCG (SEQ ID NO: 53); reverse sequence: ACCACCCTGTTGCTGTAGCCAA (SEQ ID NO: 54)). The analyses were performed using the QuantStudio7 Flex Real-time PCR System with SYBR Select Master Mix from Life Technologies Corporation. RNA expression was normalized to GAPDH and expressed as fold-changes relative to the relevant untreated control.

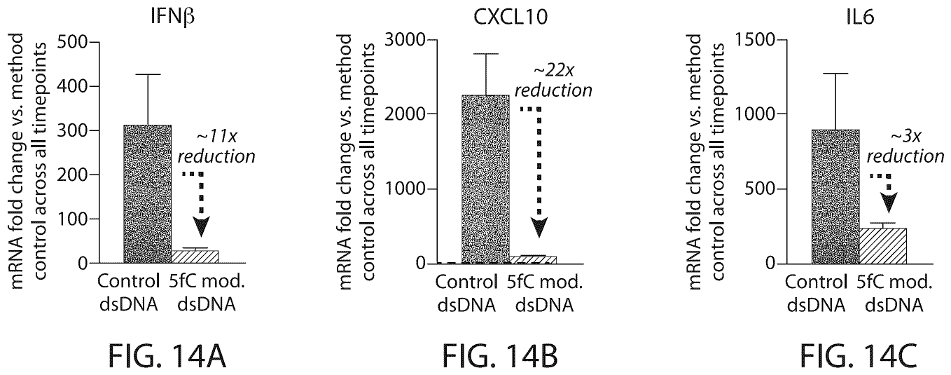

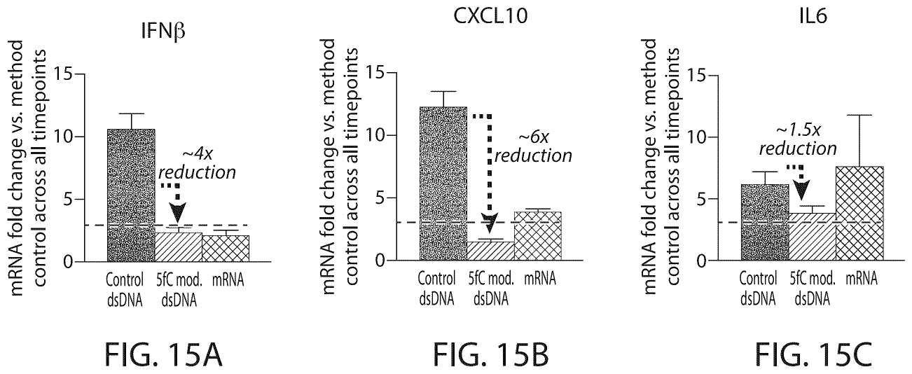

FIGS. 14A-14C and FIGS. 15A-15C show the innate immune response of HEKa cells and U937 cells to circular dsDNA molecules chemically modified with 5-formylcytosine. In both HEKa (FIGS. 14A-14C) and U937 (FIGS. 15A-15C) cells, the production of innate immune biomarkers related to the interferon response (IFNB, CXCL10) and inflammatory cytokine response (IL6) was reduced in dsDNA molecules modified with 5-formylcytosine as compared to dsDNA molecules with unmodified nucleotides. In U937 cells, the magnitude of reduction of IFNβ and CXCL10 mRNA levels was comparable to messenger RNA chemically modified with 5-methoxyuridine to reduce immunogenicity. These results demonstrate that incorporation of 5-formylcytosine into dsDNA molecules can reduce the innate immune response to DNA in multiple immune-competent cell types.

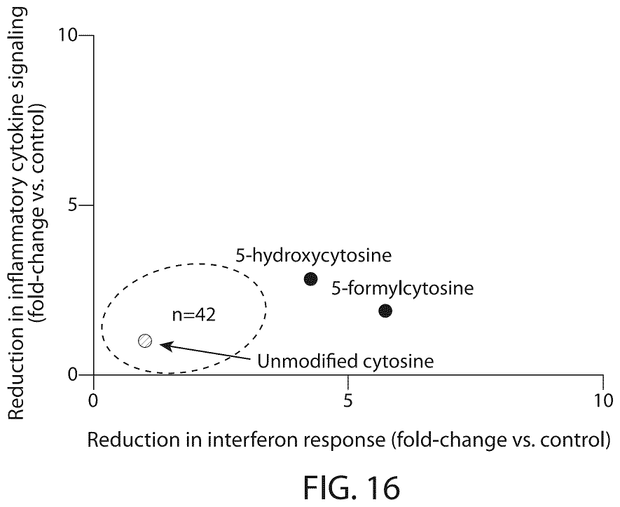

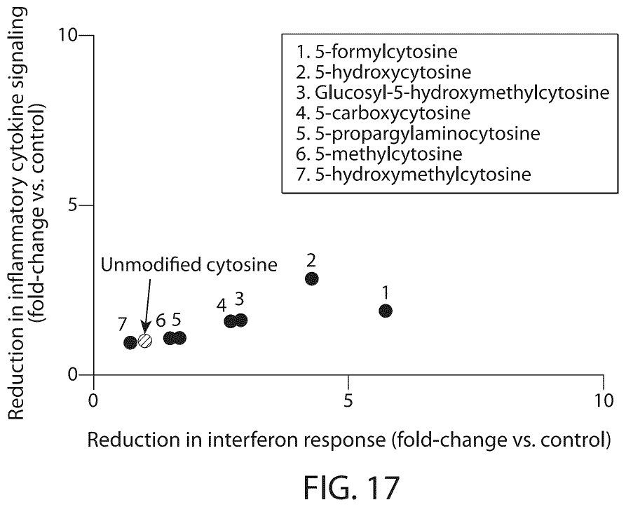

FIGS. 16 and 17 show the innate immune response of HEKa cells to linear dsDNA molecules with phosphorothioated end adapters and various modifications at the C-5 position of cytosine. For each distinct chemically modified dsDNA molecule, the innate immune response was visualized as a scatter plot in which the X-axis represents reduction in interferon signaling, as defined as the average fold-change reduction of markers IFNB and CXCL10 relative to unmodified dsDNA, and in which the Y-axis represents reduction in inflammatory cytokine signaling, as defined as the average fold-change reduction of markers IL6 and TNFa relative to unmodified dsDNA. Several specific chemical modifications at the C-5 position of cytosine, specifically 5-hydroxycytosine and 5-formylcytosine, reduced production of these markers as compared to unmodified dsDNA and other dsDNA chemical modifications (dashed-line oval; n=42 in FIG. 16). These results demonstrate that incorporation of multiple specific chemical modifications at the C-5 position of cytosine can reduce the innate immune response to DNA in an immune-competent cell line.

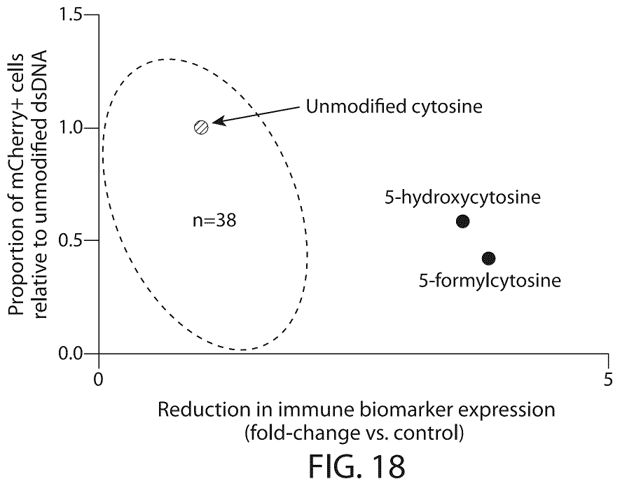

FIG. 18 shows the innate immune response to dsDNA, and reporter gene expression, of dsDNA molecules containing phosphorothioated end adapters and chemical modifications at the C-5 position of cytosine. Each distinct chemically modified dsDNA molecule was visualized as a point in a scatter plot in which the X-axis represents reduction in innate immune signaling, as defined as the average fold-change reduction of markers IFNB, CXCL10, IL6, and TNFa relative to unmodified DNA, and in which the Y-axis represents relative reporter gene expression, as defined as the proportion of mCherry+ cells relative to unmodified control DNA. Several specific C-5 cytosine modifications to dsDNA, including 5-hydroxycytosine and 5-formylcytosine, diminished cellular immune responses in HEKa cells while still retaining function relative to unmodified dsDNA and other specific dsDNA chemical modifications (dashed-line oval, n=38). These results demonstrate that incorporation of multiple chemical modifications at the C-5 position of cytosine can reduce the innate immune response to DNA while enabling protein expression in immune-competent cells.

Collectively, these results demonstrate that C-5 modifications to cytosine, such as 5-formylcytosine and 5-hydroxycytosine, can reduce the immunogenicity of dsDNA while retaining the capacity to encode a functional protein product.

### Example 8: Quantification of DNA Chemical Modifications In Vitro

This example describes the quantification of modified cytosines within chemically modified dsDNA molecules.

Purified dsDNA molecules with chemically modified cytosines (e.g. 5-formylcytosine and 5-hydroxycytosine) are prepared from a plasmid template as described in Examples 4 and 5 above. The proportion of modified cytosines is quantified by liquid chromatography coupled to tandem mass spectrometry (LC-MS/MS) as previously described in Bachman et al., 2014, Nature Chemistry volume 6 issue 12 pages 1049-1055). Briefly, DNA is degraded to nucleosides via incubation with DNA Degradase Plus (Zymo Research). Following enzymatic digestion, LC-MS/MS analysis is conducted on a mass spectrometer fitted with a liquid chromatography system. Calibration curves are generated using a mixture of synthetic standards in the ranges of 0.01-100 μM for deoxycytosine and 0.0001-1 μM for chemically modified cytosines (e.g., 5-formyl-2′deoxycytosine and 5-hydroxy-2-deoxycytosine), respectively. Samples and synthetic standards are spiked with an isotopically labeled mix of deoxycytosine and chemically modified derivatives as an internal standard. The mass spectrometer is operated in multiple reaction monitoring (MRM) mode. The ion source is electrospray in positive mode. Results are expressed as a percentage of total cytosines.

### Example 9: Validation of Chemically Modified DNA Sequences in Cells

This example describes the sequence validation of chemically modified DNAs delivered to cells.

Purified dsDNA constructs with native or chemically modified cytosines (e.g., 5-formyl-2′deoxycytosine and 5-hydroxy-2′-deoxycytosine) are prepared as described in Examples 4 and 5 and delivered to cells via lipofection as described in Example 6 above. Following transfection, DNA and RNA are simultaneously extracted from cells (AllPrep DNA/RNA Kit, Qiagen) and converted into libraries for next-generation sequencing (Illumina). Demultiplexed reads are mapped to the sequence of the chemically modified dsDNA constructs as described in Langmead and Salzberg, 2012, Nature Methods volume 9 pages 357-359. Mutations are identified via standard variant calling programs.

### Example 10: Assessment of Reporter Gene Expression In Vitro

This example demonstrates detection and quantification of gene expression using hemi-modified, covalently closed dsDNA molecules comprising loop ends. The hemi-modified dsDNA molecules comprise chemically modified cytosine nucleobases, e.g., 5-hydroxycytosine, on the sense strand, while the antisense strand is free of chemically modified nucleobases. Furthermore, the hemi-modified dsDNA molecules comprise phosphorothioate modifications near the loop ends.

Experimental DNA molecules and controls were administered via lipid transfection (lipofection). Lipofection for DNA was performed using the Lipofectamine3000 transfection reagent (#L3000001, ThermoFisher) in HEKa cells. A 1:2:3 ratio of DNA:P3000:Lipofectamine3000 was used for all DNA constructs and controls. 10,000 to 30,000 cells were pre-seeded into each well of 96-well plates one day before transfection. Transfection was performed when cells reached roughly 80 to 90% confluence. For each well of a 96-well plate, 3× Lipofectamine3000 was first diluted in 5 uL of Opti-MEM™ I Reduced Serum Medium (#31985070, ThermoFisher). DNA was diluted in 5 uL Opti-MEM™ I Reduced Serum Medium with 2×P3000 reagent. The DNA was then added into the Lipofectamine3000 containing Opti-MEM™ I Reduced Serum Medium and mixed gently by pipetting. After incubating for 15 minutes at room temperature, the DNA-Lipofectamine3000 complex was added to target cells with full culture medium in a dropwise manner to different areas of the well. The plate was gently rocked back-and-forth and side-to-side to evenly distribute the DNA-Lipofectamine3000 complex. Following transfection, cells were incubated in a CO₂ tissue culture incubator, and culture medium was changed 6 to 8 hours after transfection.

To determine expression of constructs encoding the fluorescent reporter mCherry, cells were first washed with PBS before dissociation with 0.25% Trypsin (#25200056, ThermoFisher) to get single cell suspension. Cells were then stained with the live/dead fixable yellow dead cell stain kit (#L34959, ThermoFisher) and fixed with 4% PFA (#J61899.AP, ThermoFisher). Cells were washed once with DPBS (#14190144, ThermoFisher) and then resuspended in DPBS. All flow cytometry was performed on Attune Nxt Flow Cytometer from ThermoFisher. For detection of mCherry signal, a yellow laser (wavelength 561 nm) was used for excitation and the YL2 620/15 emission filter was used. For live and death cell detection, a violet (405 nm) laser with the VL3 (603/48) filter was used. 10,000 events were recorded for each sample and data were analyzed using Flowjo V.9.0 software. Cells were first gated on FSC-A and SSC-A plot to remove cell debris. The population was further plotted on an FSC-A and FSC-H plot to circumscribe the single cell population. Cell viability was evaluated based on the signal intensity of the fixable live/dead yellow dye. Cells with low signal intensity were gated as live cells, while the population with high signal intensity was gated as dead cells. Finally, a bivariate plot between the fluorescent signal-expressing and non-expressing cells was used to determine the percentage of expressing cells in the live cell population. A distribution of expressing cells was used to determine the level of expression within each cell at 2 days post-transfection.

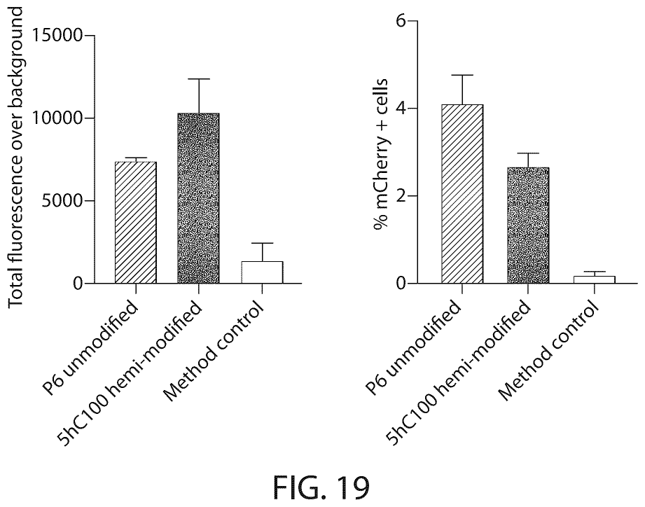

FIG. 19 shows that hemi-modified dsDNA molecules produced in a reaction using 100% 5-hydroxycytosine were functional, as defined by detectable expression of the reporter protein mCherry. The total fluorescence intensity of cells lipofected with the hemi-modified dsDNA molecules was greater than cells lipofected with control covalently closed dsDNA (comprising phosphorothioate modifications but not chemically modified nucleobases; “P6 unmodified”) and method control. These results demonstrate that hemi-modified DNAs comprising chemically modified cytosine nucleotides, e.g., comprising 5-hydroxycytosine, can be efficiently transcribed and ultimately yield a protein product in cells.

### Example 11: Assessment of Innate Immune Response in Cells In Vitro

This example demonstrates the effect of hemi-modified dsDNA molecules, as described in Example 10, on the innate immune response of cultured cells.

Experimental constructs were administered to cells as described in Example 10 above. qPCR was performed on cells to determine the RNA level of proinflammatory cytokines, including human IL6, CXCL10. Human GAPDH was used as an endogenous control for analysis. Primer sequences can be found in the Table 3. Briefly, mRNA was extracted from cells using the PicoPure RNA Isolation Kit (ThermoFisher #KIT0204). cDNA was synthesized using the RNA to cDNA EcoDry™ Premix (Oligo dT) (Takara #639542) kit. The analyses were performed using the QuantStudio7 Flex Real-time PCR System with SYBR Select Master Mix from Life Technologies Corporation. RNA expression was normalized to GAPDH and expressed as fold-change relative to the relevant vehicle control.

TABLE 4
Primer sequences used in qPCR
quantification of immune markers.

| Gene Name | 5′-Sequence-3′ |
|---|---|
| h-GAPDH-F | GTCTCCTCTGACTTCAACAGCG |
| | (SEQ ID NO: 53) |
| h-GAPDH-R | ACCACCCTGTTGCTGTAGCCAA |
| | (SEQ ID NO: 54) |
| h-IL6-F | AGACAGCCACTCACCTCTTCAG |
| | (SEQ ID NO: 43) |
| h-IL6-R | TTCTGCCAGTGCCTCTTTGCTG |
| | (SEQ ID NO: 44) |
| h-CXCL10-F | GGTGAGAAGAGATGTCTGAATCC |
| | (SEQ ID NO: 49) |
| h-CXCL10-R | GTCCATCCTTGGAAGCACTGCA |
| | (SEQ ID NO: 50) |

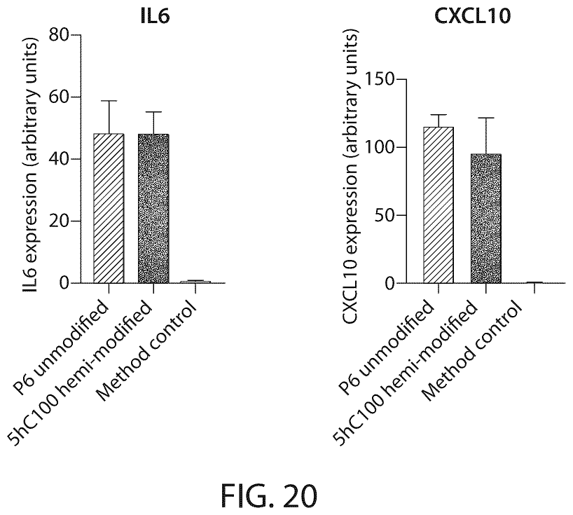

FIG. 20 shows the levels of two innate immune markers—CXCL10, a marker of interferon response, and IL6, a pro-inflammatory cytokine—for cells administered with hemi-modified dsDNA molecules produced in a reaction using 100% 5-hydroxycytosine, as compared to cells administered control covalently closed DNA (comprising phosphorothioate modifications but not chemically modified nucleobases; “P6 unmodified”).

### Example 12: Assessment of Mutations During Production of dsDNA Molecules

This Example demonstrates quantification of mutagenesis during production of dsDNA molecules comprising chemically modified cytosine nucleotides.

dsDNA molecules comprising 5-formylcytosine were produced as described in Examples 4 and 5. Specifically, plasmid DNA encoding mCherry was used a template for PCR amplification using KOD Multi & Epi polymerase (Toyobo, #KME-101). PCR reaction conditions included:
a. KOD Multi & Epi polymerase at a final concentration of 1 U per 50 l reaction.
b. 100 mM unmodified dNTP solution set (N0446, New England Biolabs), at a final concentration of 200 μM.
c. 50 μM of 5-formyl-dCTP and 150 μM unmodified dCTP (for a reaction designed for 25% incorporation of 5-formylcytosine).

The PCR products were purified using standard DNA purification columns.

The PCR products from reactions using 25% 5-formylcytosine or unmodified dCTP were sequenced using Illumina MiSeq with paired-end reads, and the profile of mutations (e.g., substitutions, deletions, and insertions) of the amplicons (which were 230 nucleotides in length) were analyzed via a custom analysis pipeline. Briefly, raw sequencing reads were validated and processed with FastQC, MultiQC, and fastp, respectively. Overlapping forward and reverse reads were merged, allowing correction of up to 2 mismatches between reads, and unmerged and unpaired reads were discarded. Merged reads that passed all filters were mapped to the reference sequence using the mem algorithm of the program bwa under default settings. A custom Python script was used to filter out mapped reads that: (1) contained soft-clipped bases or map to regions outside the known amplicon location, (2) contained CIGAR string elements besides M, I, and D, or (3) had any SAM bitwise flags set other than 16. Thereafter, custom scripts were used to evaluate mapped reads that passed filtering and to calculate error rate metrics. For each read, the number, location, and length of all base substitutions, insertions, and deletions were quantified, enabling determination of the total edit distance for each read from a given sample. A Bayesian beta-binomial model was used to estimate the error rate (all substitutions, insertions, and deletions) for each site in the reference amplicon sequence for each sample, and error rates were plotted to identify regions or bases with elevated error rates. Finally, the proportion of each of seven potential outcomes was calculated for each site within each sample: correct base, mutation to one of the other three canonical bases, mutation to an unknown base, insertion, or deletion. These outcomes were recorded in a transition matrix (i.e., a confusion matrix), as is shown in Tables 6 and 7.

The mutational profiles of the amplicons are shown in Tables 4-7. Overall, PCR products with 25% 5-formylcytosine (5fC25) exhibited a mutational profile similar to that of control PCR products (from a reaction with only unmodified dCTP).

TABLE 4
Mutational profile of PCR amplicons from a reaction using
unmodified dCTP or 25% 5-formylcytosine (5fC25).
Proportion correct: +++ ≥ 0.7; 0.7 > ++ > 0.2; − ≤ 0.2.
Substitution per kilobase: +++ ≥ 5; 5 > ++ > 1; − ≤ 1.
Insertion per kilobase: +++ ≥ 0.1; 0.1 > ++ > 0.05; − ≤ 0.05.
Deletion per kilobase: +++ ≥ 0.25; 0.25 > ++ > 0.15; − ≤ 0.15.

| | Proportion | Substitution per | Insertion per | Deletion per |
|---|---|---|---|---|
| Modification | correct | kilobase | kilobase | kilobase |
| Unmodified | +++ | − | − | − |
| 5fC25 | +++ | ++ | − | ++ |

TABLE 5
Proportion of reads with zero, one, two, or three or more errors
from sequencing PCR amplicons produced from a reaction
using unmodified dCTP or 25% 5-formylcytosine (5fC25).
Legend: +++ ≥ 0.7; 0.7 > ++ > 0.2; − ≤ 0.2.

| | | | | 3 or more |
|---|---|---|---|---|
| Modification | 0 errors | 1 error | 2 errors | errors |
| Unmodified | +++ | − | − | − |
| 5fC25 | +++ | − | − | − |

TABLE 6
Percentages of nucleotide base calling in PCR amplicons
produced using unmodified dCTP.
Each column represents the true base (adenine (A),
cytosine (C), guanine (G), or thymine (T)), and each row
represents the called base (A, C, G, T, unknown base (“N”),
deletion (“del”) or insertion (“insert”)).
Legend: ++++ ≥ 99.5%; 99.5% > +++ ≥ 0.15%;
0.15% > ++ > 0.05%; − ≤ 0.05%.

| | | True base | | | |
|---|---|---|---|---|---|
| | | A | C | G | T |
| Called base | A | ++++ | − | − | − |
| | C | − | ++++ | − | − |
| | G | ++ | − | ++++ | − |
| | T | − | − | − | ++++ |
| | N | − | − | − | − |
| | Del | − | − | − | − |
| | Insert | − | − | − | − |

TABLE 7
Percentages of nucleotide base calling in PCR
amplicons produced using 25% 5-formylcytosine (5fC25).
Each column represents the true base (adenine (A),
cytosine (C), guanine (G), or thymine (T)), and each row
represents the called base (A, C, G, T, unknown base (“N”),
deletion (“del”) or insertion (“insert”)).
Legend: ++++ ≥ 99.5%; 99.5% > +++ ≥ 0.15%;
0.15% > + > 0.05%; − ≤ 0.05%.

| | | True base | | | |
|---|---|---|---|---|---|
| | | A | C | G | T |
| Called base | A | ++++ | − | ++ | − |
| | C | − | ++++ | − | ++ |
| | G | ++ | − | ++++ | − |
| | T | − | ++ | − | ++++ |
| | N | − | − | − | − |
| | Del | − | − | − | − |
| | Insert | − | − | − | − |

Mutational Rate from Transcription of dsDNA Molecules Comprising Chemically Modified Cytosine Nucleotides

To quantify the mutational rates following transcription of dsDNA comprising chemically modified cytosine nucleotides in cells, circular dsDNA molecules were produced as described in Example 5. The circular dsDNA molecules comprising chemically modified cytosine nucleotides or control (produced in a reaction with unmodified dCTP) were administered via lipid transfection to HEKa cells, as described in Example 10. Twenty-four hours post-transfection, RNA was extracted from the cells using the PicoPure RNA Isolation Kit (ThermoFisher #KIT0204) and converted into cDNA using the SuperScript IV Kit with ezDNase Enzyme (ThermoFisher, 18091300). cDNA was converted into RNA-seq libraries with the NEBNext Kit (New England Biolabs, E7645L), using Illumina i5 and i7 primers (10 μM).

The mutational profiles of the extracted RNA were quantified as described for PCR amplicons above and are shown in Table 8. Overall, the mutational profiles of extracted RNA were proportional between cells transfected with circular dsDNA molecules produced with 5-formylcytosine at 25% incorporation, and those transfected with control circular dsDNA molecules produced using unmodified dCTP.

TABLE 8
Mutational profile of RNA extracted from cells
transfected with circular dsDNA molecules produced
using unmodified dCTP or 25% 5-formylcytosine.
Proportion correct: +++ ≥ 0.7; 0.7 > ++ > 0.2; − ≤ 0.2.
Substitution per kilobase: +++ ≥ 5; 5 > ++ > 1; − ≤ 1.
Insertion per kilobase: +++ ≥ 0.1; 0.1 > ++ > 0.05; − ≤ 0.05.
Deletion per kilobase: +++ ≥ 0.25; 0.25 > ++ > 0.15; − ≤ 0.15.

| | Proportion | Substitution | Insertion per | Deletion per |
|---|---|---|---|---|
| Modification | correct | per kilobase | kilobase | kilobase |
| Unmodified | +++ | − | ++ | ++ |
| 5fC25 | +++ | ++ | ++ | +++ |

All publications, patents, and patent applications cited herein are incorporated by reference in their entirety to the same extent as if each individual publication, patent, or patent application was specifically and individually indicated to be incorporated by reference. In the event of a conflict between a term herein and a term in an incorporated reference, the term herein controls.

1. A dsDNA molecule comprising:
a promoter sequence and a therapeutic payload sequence operably linked to the promoter sequence, and
a chemically modified cytosine nucleotide situated in the therapeutic payload sequence comprising the structure of Formula I: 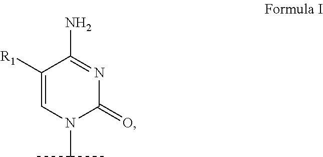
wherein R₁ is selected from the group consisting of —OH; -aldehyde; -carboxylic acid; -alkyl; —(CH₂)ₘOR₂, m=1-3 and R₂=H or a sugar molecule; and -propargylamino.

2. The dsDNA molecule of claim 1, wherein R₁ is selected from the group consisting of —OH; —CHO; —COOH; -alkyl; —(CH₂)ₘOR₂, m=1-3 and R₂=H or a sugar molecule; and -propargylamino, wherein the alkyl group includes one to six carbons.

3. The dsDNA molecule of claim 1, wherein R₁ is selected from the group consisting of —OH; —CHO; —COOH; —CH₂OR₃, R₃=H or glucose; -methyl; and -propargylamino.

4. The dsDNA molecule of claim 1, wherein the chemically modified cytosine nucleotide comprises 5-formylcytosine, 5-hydroxycytosine, 5-carboxycytosine, 5-propargylaminocytosine, 5-methylcytosine, 5-hydroxymethylcytosine, or glucosyl-5-hydroxymethylcytosine.

5. The dsDNA molecule of claim 1, wherein the dsDNA molecule is circular or linear.

6. The dsDNA molecule of claim 1, wherein the dsDNA molecule is closed-ended linear.

7. The dsDNA molecule of claim 1, wherein at least 1%, at least 5%, at least 10%, at least 15%, at least 20%, at least 25%, at least 50%, or at least 75% of cytosine positions in the dsDNA molecule comprise the chemically modified cytosine nucleotide.

8. The dsDNA molecule of claim 1, which comprises one, two, or all of:
i) a heterologous functional sequence;
ii) a maintenance sequence; or
iii) an origin of replication.

9. The dsDNA molecule of claim 1, which, when contacted to HEKa cells, results in one or more of:
a lower level of IFNβ mRNA compared to a control DNA molecule,
a lower level of CXCL10 mRNA compared to a control DNA molecule, or
a lower level of IL6 mRNA compared to a control DNA molecule,
wherein the control DNA molecule comprises the same sequence, same strandedness, and same circular or linear character as the dsDNA molecule, but comprises unmodified cytosine nucleotides in place of the chemically modified cytosine nucleotides.

10. The dsDNA molecule of claim 1, which, when contacted to HepG2 cells, results in expression of the therapeutic payload sequence at a level at least 50%, at least 60%, at least 70%, or at least 75% of the expression of the therapeutic payload sequence of a control DNA, wherein the control DNA molecule comprises the same sequence, same strandedness, and same circular or linear character as the dsDNA molecule, but comprises unmodified cytosine nucleotides in place of the chemically modified cytosine nucleotides.

11. The dsDNA molecule of claim 1, wherein the dsDNA molecule is linear and comprises:
a) an upstream exonuclease-resistant DNA end form;
b) a double stranded region; and
c) a downstream exonuclease-resistant DNA end form.

12. A double stranded DNA (dsDNA) molecule comprising:
a chemically modified cytosine nucleotide chosen from 5-hydroxycytosine or glucosyl-5-hydroxymethylcytosine,
wherein the dsDNA molecule is closed-ended linear DNA.

13. The dsDNA molecule of claim 12, which comprises a therapeutic payload sequence.

14. A pharmaceutical composition comprising the dsDNA molecule of claim 1.

15. A pharmaceutical composition comprising the dsDNA molecule of claim 13.

16. A method of making or manufacturing a double stranded DNA (dsDNA) molecule, the method comprising:
(a) providing a composition comprising a DNA template, a forward primer, a reverse primer, a DNA polymerase, unmodified deoxyribose nucleotides, and a chemically modified cytosine nucleotide having a substitution other than hydrogen at carbon 5 of the cytosine; and
(b) performing a polymerase chain reaction on the composition of (a),
thereby making or manufacturing the dsDNA molecule.

17. A dsDNA molecule produced by the method of claim 16.

18. A method of expressing a therapeutic payload in a target cell, the method comprising:
(i) providing a target cell comprising the dsDNA molecule of claim 1; and
(ii) maintaining the cell under conditions suitable for expressing a therapeutic payload from the therapeutic payload sequence of the dsDNA molecule;
thereby expressing the therapeutic payload in the target cell.

19. A method of expressing a therapeutic payload in a target cell, the method comprising:
(i) providing a target cell comprising the dsDNA molecule of claim 13; and
(ii) maintaining the cell under conditions suitable for expressing a therapeutic payload from the therapeutic payload sequence of the dsDNA molecule;
thereby expressing the therapeutic payload in the target cell.

20. A method of delivering a therapeutic payload to a target cell, the method comprising:
introducing into a target cell the dsDNA molecule of claim 1, wherein the therapeutic payload sequence encodes a therapeutic payload;
thereby delivering the therapeutic payload to the target cell.

21. A method of modulating a biological activity in a target cell, the method comprising:
(i) providing a target cell comprising the dsDNA molecule of claim 1, wherein the therapeutic payload sequence encodes a therapeutic payload that modulates a biological activity in the target cell; and
(ii) maintaining the cell under conditions suitable for expressing the therapeutic payload from the dsDNA molecule;
thereby modulating the biological activity in the target cell.

22. A method of modulating a biological activity in a target cell, the method comprising:
(i) providing a target cell comprising the dsDNA molecule of claim 13, wherein the therapeutic payload sequence encodes a therapeutic payload that modulates a biological activity in the target cell; and
(ii) maintaining the cell under conditions suitable for expressing the therapeutic payload from the dsDNA molecule;
thereby modulating the biological activity in the target cell.

23. A method of treating a cell, tissue or subject in need thereof, the method comprising:
administering to the cell, tissue or subject the dsDNA molecule of claim 1;
thereby treating cell, tissue or the subject.

24. A method of treating a cell, tissue or subject in need thereof, the method comprising:
administering to the cell, tissue or subject the dsDNA molecule of claim 13;
thereby treating cell, tissue or the subject.


SEQUENCE LISTING (93 sequences)

SEQ ID NO: 1 - DNA, 21 bp, Betapolyomavirus macacae
  source 1..21: mol_type = genomic DNA
cccaagaagaagaggaaagtc

SEQ ID NO: 2-25 - skipped (24 entries)

SEQ ID NO: 26 - DNA, 141 bp, Adeno-associated virus 2
  source 1..141: mol_type = genomic DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 27-36 - skipped (10 entries)

SEQ ID NO: 37 - DNA, 1179 bp, synthetic construct
  source 1..1179: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 38 - DNA, 711 bp, synthetic construct
  source 1..711: mol_type = other DNA
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 39 - DNA, 156 bp, Homo sapiens
  source 1..156: mol_type = genomic DNA
tataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacata

SEQ ID NO: 40 - DNA, 12 bp, synthetic construct
  source 1..12: mol_type = other DNA
ccgtggtccttc

SEQ ID NO: 41 - DNA, 24 bp, synthetic construct
  source 1..24: mol_type = other DNA
cttggattcctacaaagaagcagc

SEQ ID NO: 42 - DNA, 22 bp, synthetic construct
  source 1..22: mol_type = other DNA
tcctccttctggaactgctgca

SEQ ID NO: 43 - DNA, 22 bp, synthetic construct
  source 1..22: mol_type = other DNA
agacagccactcacctcttcag

SEQ ID NO: 44 - DNA, 22 bp, synthetic construct
  source 1..22: mol_type = other DNA
ttctgccagtgcctctttgctg

SEQ ID NO: 45-46 - skipped (2 entries)

SEQ ID NO: 47 - DNA, 22 bp, synthetic construct
  source 1..22: mol_type = other DNA
ctcttctgcctgctgcactttg

SEQ ID NO: 48 - DNA, 22 bp, synthetic construct
  source 1..22: mol_type = other DNA
atgggctacaggcttgtcactc

SEQ ID NO: 49 - DNA, 23 bp, synthetic construct
  source 1..23: mol_type = other DNA
ggtgagaagagatgtctgaatcc

SEQ ID NO: 50 - DNA, 22 bp, synthetic construct
  source 1..22: mol_type = other DNA
gtccatccttggaagcactgca

SEQ ID NO: 51 - DNA, 23 bp, synthetic construct
  source 1..23: mol_type = other DNA
aagttgtctgtgtgcgcaaatcc

SEQ ID NO: 52 - DNA, 24 bp, synthetic construct
  source 1..24: mol_type = other DNA
ccattccagaaaagccacagtttt

SEQ ID NO: 53 - DNA, 22 bp, synthetic construct
  source 1..22: mol_type = other DNA
gtctcctctgacttcaacagcg

SEQ ID NO: 54 - DNA, 22 bp, synthetic construct
  source 1..22: mol_type = other DNA
accaccctgttgctgtagccaa

SEQ ID NO: 55 - DNA, 28 bp, synthetic construct
  source 1..28: mol_type = other DNA
tatcagcacacaattgcccattatacgc

SEQ ID NO: 56 - DNA, 28 bp, synthetic construct
  source 1..28: mol_type = other DNA
gcgtataatgggcaattgtgtgctgata

SEQ ID NO: 57 - DNA, 28 bp, synthetic construct
  source 1..28: mol_type = other DNA
tatcagcacacaatagtccattatacgc

SEQ ID NO: 58 - DNA, 28 bp, synthetic construct
  source 1..28: mol_type = other DNA
gcgtataatggactattgtgtgctgata

SEQ ID NO: 59 - DNA, 32 bp, synthetic construct
  source 1..32: mol_type = other DNA
aattctcctccccaccttccccaccctcccca

SEQ ID NO: 60 - DNA, 21 bp, synthetic construct
  source 1..21: mol_type = other DNA
acctatttcagcatactacgc

SEQ ID NO: 61 - DNA, 21 bp, synthetic construct
  source 1..21: mol_type = other DNA
gcgtagtatgctgaaataggt

SEQ ID NO: 62 - DNA, 44 bp, synthetic construct
  source 1..44: mol_type = other DNA
cacacaattgcccattatacgcgcgtataatgggcaattgtgtg

SEQ ID NO: 63 - DNA, 14 bp, synthetic construct
  source 1..14: mol_type = other DNA
taaatataatttaa

SEQ ID NO: 64 - DNA, 14 bp, synthetic construct
  source 1..14: mol_type = other DNA
ttaaattatattta

SEQ ID NO: 65 - DNA, 14 bp, synthetic construct
  source 1..14: mol_type = other DNA
aatatataatctaa

SEQ ID NO: 66 - DNA, 14 bp, synthetic construct
  source 1..14: mol_type = other DNA
ttagattatatatt

SEQ ID NO: 67 - DNA, 14 bp, synthetic construct
  source 1..14: mol_type = other DNA
tatttattatcttt

SEQ ID NO: 68 - DNA, 14 bp, synthetic construct
  source 1..14: mol_type = other DNA
aaagataataaata

SEQ ID NO: 69 - DNA, 32 bp, synthetic construct
  source 1..32: mol_type = other DNA
atataatttttaattagtatagaatatgttaa

SEQ ID NO: 70 - DNA, 32 bp, synthetic construct
  source 1..32: mol_type = other DNA
ttaacatactctatactaattaaaaattatat

SEQ ID NO: 71 - DNA, 26 bp, synthetic construct
  source 1..26: mol_type = other DNA
tataatttgatattagtacaaatccc

SEQ ID NO: 72 - DNA, 26 bp, synthetic construct
  source 1..26: mol_type = other DNA
gggatttgtactaatatcaaattata

SEQ ID NO: 73 - DNA, 27 bp, synthetic construct
  source 1..27: mol_type = other DNA
atataatatttatttagtacaaagttc

SEQ ID NO: 74 - DNA, 27 bp, synthetic construct
  source 1..27: mol_type = other DNA
gaactttgtactaaataaatattatat

SEQ ID NO: 75 - DNA, 27 bp, synthetic construct
  source 1..27: mol_type = other DNA
atataattttttattagtatagagtat

SEQ ID NO: 76 - DNA, 27 bp, synthetic construct
  source 1..27: mol_type = other DNA
atactctatactaataaaaaattatat

SEQ ID NO: 77 - DNA, 16 bp, synthetic construct
  source 1..16: mol_type = other DNA
tagtataaaaaactgt

SEQ ID NO: 78 - DNA, 16 bp, synthetic construct
  source 1..16: mol_type = other DNA
acagttttttatacta

SEQ ID NO: 79 - DNA, 16 bp, synthetic construct
  source 1..16: mol_type = other DNA
tagtatacaaaagatt

SEQ ID NO: 80 - DNA, 16 bp, synthetic construct
  source 1..16: mol_type = other DNA
aatcttttgtatacta

SEQ ID NO: 81 - DNA, 16 bp, synthetic construct
  source 1..16: mol_type = other DNA
tagtatatatatctct

SEQ ID NO: 82 - DNA, 16 bp, synthetic construct
  source 1..16: mol_type = other DNA
agagatatatatacta

SEQ ID NO: 83 - DNA, 16 bp, synthetic construct
  source 1..16: mol_type = other DNA
tagtataaaaaaaatt

SEQ ID NO: 84 - DNA, 16 bp, synthetic construct
  source 1..16: mol_type = other DNA
aattttttttatacta

SEQ ID NO: 85 - DNA, 46 bp, unidentified
  source 1..46: mol_type = unassigned DNA
ctggggactttccagcctggggactttccagctgggactttccagg

SEQ ID NO: 86 - DNA, 47 bp, synthetic construct
  source 1..47: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
  modified_base 6: mod_base = OTHER; note = Nucleotide with phosphorothioate linkage
  modified_base 40: mod_base = OTHER; note = Nucleotide with phosphorothioate linkage
  modified_base 41: mod_base = OTHER; note = Nucleotide with phosphorothioate linkage
  modified_base 42: mod_base = OTHER; note = Nucleotide with phosphorothioate linkage
  modified_base 43: mod_base = OTHER; note = Nucleotide with phosphorothioate linkage
  modified_base 44: mod_base = OTHER; note = Nucleotide with phosphorothioate linkage
  modified_base 45: mod_base = OTHER; note = Nucleotide with phosphorothioate linkage
cccgagcaggatcgagccacacgtactacgctcgatcctgctcgggt

SEQ ID NO: 87 - DNA, 48 bp, synthetic construct
  source 1..48: mol_type = other DNA
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 41: mod_base = OTHER; note = Nucleotide with phosphorothioate linkage
  modified_base 42: mod_base = OTHER; note = Nucleotide with phosphorothioate linkage
  modified_base 43: mod_base = OTHER; note = Nucleotide with phosphorothioate linkage
  modified_base 44: mod_base = OTHER; note = Nucleotide with phosphorothioate linkage
  modified_base 45: mod_base = OTHER; note = Nucleotide with phosphorothioate linkage
  modified_base 46: mod_base = OTHER; note = Nucleotide with phosphorothioate linkage
acccgagcaggatcgagccacacgtactacgctcgatcctgctcgggt

SEQ ID NO: 88 - DNA, 56 bp, synthetic construct
  source 1..56: mol_type = other DNA
tatcagcacacaattgcccattatacgcgcgtataatggactattgtgtgctgata

SEQ ID NO: 89 - DNA, 56 bp, synthetic construct
  source 1..56: mol_type = other DNA
tatcagcacacaatagtccattatacgcgcgtataatgggcaattgtgtgctgata

SEQ ID NO: 90 - DNA, 18 bp, synthetic construct
  source 1..18: mol_type = other DNA
ccgtggtccttcggtacc

SEQ ID NO: 91 - DNA, 18 bp, synthetic construct
  source 1..18: mol_type = other DNA
ggtaccgaaggaccacgg

SEQ ID NO: 92 - DNA, 18 bp, synthetic construct
  source 1..18: mol_type = other DNA
ggtaccgccttccaccgg

SEQ ID NO: 93 - DNA, 18 bp, synthetic construct
  source 1..18: mol_type = other DNA
ccggtggaaggcggtacc